SEQUENCE LISTING (10 sequences)

SEQ ID NO: 1 - protein, 585 aa, Homo sapiens
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 2 - protein, 585 aa, Pan troglodytes
MTPPRLFWVWLLVAGTQGVNDGDMRLADGGATNQGRVEIFYRGQWGTVCDNLWDLTDASVVCRALGFENATQALGRAAFGQGSGPIMLDEVQCMGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPRDPSFQTPLDLYAYAVATGDALLEKLCLQFLAWNFEALTQAEAWPSVPTDLLQLLLPRSDLAVPSELALLKAVDTWSWGERASHEEVEDLVEKIRFPMMLPEELFELQFNLSLYWSHEALFQKKTLQALEFHTVPFQLLARYKGLNLTEDTYKPRIYTSPTWSASVTDSSWSARKSQLVYQSRRGPLVKYSSNYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDINSSKSTSSFPCPAGHFNGFRTVIRPFYLTNSSGVD

SEQ ID NO: 3 - protein, 559 aa, Canis lupus famiilaris
MALPLVLWMCLLVAGTQGVKDGDMRLANGDTANEGRVEIFYSGRWGTVCDNLWDLMDASVVCRALGFENATEALGGAAFGPGKGPIMLDEVECTGTEPSLANCTSLGWMKSNCRHNQDAGVVCSNETRGAHTLDLSGELPAALEQIFDSQRGCDLSIRVKVKDQEEEGPHFCAHRLILAANPEAQALCKAPGSTVTMEVDAECLPVVRDFIRYLYSRRLDISLTSVKCFHKLASAYEAQQLQSFCASLFAILLPEDPSFQAPLDLYAYALATQDPVLEELCVQFLAWNFEGLTQATAWPRVPTALLQLLLSRSELAVPSELALLTALDVWSQERRPSHGEVARLVDKVRFPMMLPEHLFELQFNLSLYWSHEALFQKKILQALEFHTVPFRLLAQHRGLNLTEDAYQPRLYTSPTWSASVSRSSSRYWNYPYQSFQTPQHPSFLFQNKYISWSLVYLPTVQSCWNYGFSCSSDEVPLLGLSKSDYSDPTIGYENKALMRCGGRFVADVTDFEGQKALIPSALGTNSSRRPSLFPCLGGSFSSFQVVIRPFYLTNSSDVD

SEQ ID NO: 4 - protein, 555 aa, Bos taurus
MAPLRLFWIWLLVVGTRGVKDGDMRLADGGSANQGRVEIYYNGQWGTVCENMWDLTDASVVCRALGFQNATEALGGAAFGPGYGPIMLDEVRCTGTEPSLANCSSLGWMRSNCRHDKDASVICTNETRGVYTLDLSGELPAALEQIFESQKGCDLFITVKVREEDEIAMCAHKLILSTNPEAHGLWKEPGSRVTMEVDAECVPVVKDFIRYLYSRRIDVSLSSVKCLHKFASAYQAKQLQSYCGHLFAILIPQDPSFWTPLELYAYALATRDTVLEEICVQFLAWNFGALTQAEAWPSVPPALLQGLLSRTELVVPSELVLLLAVDKWSQERHTSHKEVEALVGQVRFPMMPPQDLFSLQFNLSLYWSHEALFQKKILQALEFHTVPFELLAQYWGLNLTEGTYQPRLYTSPTWSQSVMSSSYNPSRSFQTPQHPSFLFHDSSVSWSFVYLPTLQSCWNYGFSCSSDDPPLLALSKSSYSKSNPTIGYENRALLHCEGSFVVDVIDFKGWKALVPSALATNSSRSTSLFPCPSGVFSRFQVVIRPFYLTNSTDMD

SEQ ID NO: 5 - protein, 577 aa, Mus musculus
MALLWLLSVFLLVPGTQGTEDGDMRLVNGASANEGRVEIFYRGRWGTVCDNLWNLLDAHVVCRALGYENATQALGRAAFGPGKGPIMLDEVECTGTESSLASCRSLGWMVSRCGHEKDAGVVCSNDTTGLHILDLSGELSDALGQIFDSQQGCDLFIQVTGQGYEDLSLCAHTLILRTNPEAQALWQVVGSSVIMRVDAECMPVVRDFLRYFYSRRIEVSMSSVKCLHKLASAYGATELQDYCGRLFATLLPQDPTFHTPLDLYAYARATGDSMLEDLCVQFLAWNFEPLTQSESWSAVPTTLIQALLPKSELAVSSELDLLKAVDQWSTETIASHEDIERLVEQVRFPMMLPQELFELQFNLSLYQDHQALFQRKTMQALEFHTVPVEVLAKYKGLNLTEDTYKPRLYTSSTWSSLVMASTWRAQRYEYNRYNQLYTYGYGSVARYNSYQSFQTPQHPSFLFKDKQISWSATYLPTMQSCWNYGFSCTSNELPVLGLTTSSYSNPTIGYENRVLILCGGYSVVDVTSFEGSKAPIPTALDTNSSKTPSLFPCASGAFSSFRVVIRPFYLTNSTDMV

SEQ ID NO: 6 - protein, 574 aa, Ratus norvegicus
MALLWLLSVFLLVPGTQGAKDGDMRLVNGASASEGRVEIFYRGRWGTVCDNLWNLLDAHVVCRALGYENATQALSRAAFGPGKGPIMLDEVECTGNESSLANCSSLGWMVSHCGHEKDAGVVCSNDSRGIHILDLSGELPDALGQIFDSQQDCDLFIQVTGQGHGDLSLCAHTLILRTNPEAQALWQVVGSSVIMRVDAECMPVVRDFLRYFYSRRIEVSMSSVKCLHKLASAYGATELQGYCGRLFVTLLPQDPTFHTPLELYEYAQATGDSVLEDLCVQFLAWNFEPLTQAEAWLSVPNALIQALLPKSELAVSSELDLLKAVDQWSTATGASHGDVERLVEQIRFPMMLPQELFELQFNLSLYQGHQALFQRKTMEALEFHTVPLKVLAKYRSLNLTEDVYKPRLYTSSTWSSLLMAGAWSTQSYKYRQFYTYNYGSQSRYSSYQNFQTPQHPSFLFKDKLISWSATYLPTIQSCWNYGFSCTSDELPVLGLTTSSYSDPTIGYENKALILCGGYSVVDVTTFIGSKAPIPGTQETNSSKTPSLFPCASGAFSSFREVIRPFYLTNSTDTE

SEQ ID NO: 7 - DNA, 18 bp, Artificial Sequence
  Description of Artificial Sequence Forward Primer
agcagccacacccagaag

SEQ ID NO: 8 - DNA, 17 bp, Artificial Sequence
  Description of Artificial Sequence Reverse Primer
gaggaggctccacacgg

SEQ ID NO: 9 - DNA, 20 bp, Artificial Sequence
  Description of Artificial Sequence Forward Primer
cgccgctagaggtgaaattc

SEQ ID NO: 10 - DNA, 19 bp, Artificial Sequence
  Description of Artificial Sequence Reverse Primer
ttggcaaatgctttcgctc

(19) United States
(12) Patent Application Publication
(10) Pub. No.: US 2014/0234320 A1
(43) Pub. Date: Aug. 21, 2014

(54) MODULATORS OF 4-1BB AND IMMUNE RESPONSES

(71) Applicants: Michael Croft, San Diego, CA (US); Shravan Madireddi, San Diego, CA (US)

(72) Inventors: Michael Croft, San Diego, CA (US); Shravan Madireddi, San Diego, CA (US)

(73) Assignee: LA JOLLA INSTITUTE FOR ALLERGY AND IMMUNOLOGY, San Diego, CA (US)

(21) Appl. No.: 14/127,134

(22) PCT Filed: Jun. 20, 2012
(86) PCT No.: PCT/US12/43383

§ 371 Date: Apr. 24, 2014

Related U.S. Application Data
(60) Provisional application No. 61/499,052, filed on Jun. 20, 2011.

(51) Int. Cl. C07K 16/24; C12N 15/115; C07K 16/28; A61K 38/02; C12N 15/113

(52) U.S. Cl. CPC C07K 16/241; A61K 38/02; C12N 15/1136; C07K 16/2851; C12N 15/115
USPC 424/139.1; 424/184.1; 424/192.1

(57) ABSTRACT

The invention provides peptides and fragments, methods and uses based upon modulating the binding or interaction between 4-1BB and galectins such as Galectin-9. Modulating such binding or interaction between 4-1BB and a galectin, such as Galectin 9. modulates an immune response.

### RELATED APPLICATIONS

This application is a U.S. National Phase of International Application No. PCT/US2012/043383, filed Jun. 20, 2012, which designated the U.S. and that International Application was published under PCT Article 21(2) in English, and claims priority to U.S. Provisional Application No. 61/499,052, filed Jun. 20, 2011, all of which applications are incorporated herein by reference in their entirety.

### GOVERNMENT SUPPORT

This invention received government support from the National Institutes Health grants P01 AI089624, R21 AI085291, R01 AIO42944. The government has certain rights in the invention.

### FIELD OF THE INVENTION

The present invention relates generally to a method for modulating an immune response comprising modulating the binding between 4-1BB and a galectin and methods for screening for and identifying agents that can modulate the binding between 4-1BB and a galectin.

### INTRODUCTION

Interactions between members of the tumor-necrosis factor receptor (TNFR) super-family control many lymphoid cell types and a number of molecules in this family of proteins are being targeted for therapy of immune disease. 4-1BB is one member of the family expressed on activated T cells, NK cells, dendritic cells, and myeloid progenitors.

4-1BB (CD137, ILA, TNFRSF9) is a TNFR super-family molecule that can be induced on many cells types including T cells, NK cells, dendritic cells, and myeloid progenitors. 4-1BB activity may be involved in many immune-mediated autoimmune and inflammatory diseases. It is also a target for cancer immunotherapy. 4-1BB can have both pro- and anti-inflammatory activities, but it is not clear how these are brought about and what is the relevant ligand with which 4-1BB interacts when exerting its contrasting activities.

### SUMMARY

The invention is based upon, inter alia, the discovery of the interaction between 4-1BB and galectins such as Galectin-9. Interactions between 4-1BB and its recognized ligand 4-BBL, a member of the TNF super-family also known as TNFSF9, cannot explain all of the functional activities attributed to 4-1BB. The discovery of the interaction between 4-1BB and Galectin-9 provides a previously unknown way in which to modulate effector functions of the interaction.

Accordingly, the invention provides compounds, uses and methods for modulating an immune response, by modulating binding between 4-1BB and Galectin-9.

In one embodiment, invention uses and methods of modulating an immune response include contacting 4-1BB or a galectin with an agent that modulates binding of 4-1BB to the galectin, thereby modulating an immune response. In particular aspects, a use or method includes contacting 4-1BB with an agent that modulates binding of 4-1BB to the galectin; or contacting a galectin with an agent that modulates binding of 4-1BB to the galectin. In additional aspects, an agent decreases, reduces, inhibits, suppresses or disrupts binding of 4-1 BB to the galectin. In further aspects, an agent increases, enhances, stimulates, or promotes binding of 4-1BB to the galectin.

Agents include, but are not limited to agents that bind to the galectin, agents that bind to 4-1BB ligand (4-1BBL), and agents that bind to Tim-3 or binds to CD44. Such agents include antibodies and antibody fragments that bind to 4-1BB, a galectin, 4-1BB ligand (4-1BBL), Tim-3 or CD44 polypeptide sequence.

Antibodies include polyclonal, monoclonal and fragments of monoclonal antibodies. Such antibodies and fragments include a mammalian antibody or a fragment of a mammalian antibody, such as a human, humanized, primatized or chimeric antibody or fragment of a human, humanized, primatized and chimeric antibodies and fragments thereof.

Agents also include peptides and fragments of a 4-1BB, galectin, Galectin-9, 4-1BB ligand (4-1BBL), Tim-3 or CD44 polypeptide sequences. Peptides and fragments of 4-1BB, galectin, Galectin-9, 4-1BB ligand (4-1BBL), Tim-3 and CD44 polypeptide sequences include mammalian sequences, such as a primate (human) or rodent (murine) sequences. In particular aspects, a 4-1BB peptide or fragment includes or consists of cysteine rich domains (CRDs) 2 and/or 3 of 4-1BB, or a subsequence of cysteine rich domains (CRDs) 2 and/or 3 of 4-1BB. In additional particular aspects, a 4-1BBL peptide or fragment comprises or consists of from about residues 49-254 of 4-1BBL, 115 to 227 of 4-1BBL, from about 104-309 of 4-1BBL, 105-309 of 4-1BBL, or 106-309 of 4-1BBL, or a subsequence thereof. In further particular aspects, a 4-1BBL peptide or fragment includes or consists of from about residues 49-254 of 4-1BBL, 115 to 227 of 4-1BBL, from about 104-309 of 4-1BBL, 105-309 of 4-1BBL, or 106-309 of 4-1BBL, or a subsequence thereof.

In accordance with the invention, galectins include Galectin-9. Agents therefore include galectins, such as Galectin-9, and peptides and fragments of galectins such as Galectin-9. In particular aspects, a galectin or Galectin-9 peptide or fragment includes or consists of a carbohydrate binding/recognition domain of galectin or Galectin-9, or a subsequence of a carbohydrate binding/recognition domain (CBD) of galectin or Galectin-9.

In more particular aspects, a galectin or Galectin-9 peptide or fragment includes or consists of a CBD1 sequence or a subsequence thereof, or CBD2 sequence or a subsequence thereof. In further particular aspects, a Galectin-9 peptide or fragment that binds to Tim-3 includes or consists of an N-terminal sequence portion of Galectin-9 from residues 50-58, 77-80, and/or 122-131; or a C-terminal sequence portion of Galectin-9 from residues 290-295, 310-314, and/or 326-336, or a subsequence thereof.

Agents also include Tim-3 peptides and fragments thereof. In particular aspects, a Tim-3 peptide or fragment binds to a galectin, such as Galectin-9.

Agents that comprise protein, polypeptide or peptide sequences such as antibodies and peptides and fragments of a 4-1BB, galectin, Galectin-9, 4-1BB ligand (4-1BBL), Tim-3 or CD44 polypeptide sequence can be of various lengths. In particular embodiments, a peptide or fragment has a sequence length from about 10 to about 20, L- or D-amino acids, about 20 to about 50 L- or D-amino acids, about 50 to about 100 L- or D-amino acids, about 100 to about 150 L- or D-amino acids, or from about 150 to about 200 L- or D-amino acids.

Agents further include inhibitory nucleic acids. Such inhibitory nucleic acids include those that reduce expression or activity of 4-1BB, a galectin, 4-1BB ligand, Tim-3 or CD44. Inhibitory nucleic acids include single and double strand RNA and DNA nucleic acids that binds to genomic, transcribed or mRNA sequence of any of 4-1BB, the galectin, 4-1BB ligand, Tim-3 or CD44.

Agents moreover include aptamers and small molecules. Such aptamers and small molecules also can bind to 4-1BB, a galectin, 4-1BB ligand, Tim-3 or CD44 and reduce expression or activity of 4-1BB, a galectin, 4-1BB ligand, Tim-3 or CD44.

Invention uses and methods of modulating an immune response include decreasing, reducing, inhibiting, suppressing, limiting or controlling an undesirable or aberrant immune response, disorder or disease, an inflammatory response, disorder or disease, inflammation, or an autoimmune response, disorder or disease, or an adverse symptom of an undesirable or aberrant immune response, disorder or disease, an inflammatory response, disorder or disease, inflammation, or an autoimmune response, disorder or disease. Invention uses and methods of modulating an immune response also include increasing, stimulating, enhancing, promoting, inducing or activating an immune response, inflammatory response or inflammation.

Invention uses and methods of modulating an immune response in a subject include administering an agent that modulates binding of 4-1BB to a galectin to the subject, thereby modulating the immune response in the subject. Such subjects include but are not limited to: a subject that has or has had an undesirable or aberrant immune response, disorder or disease, an inflammatory response, disorder or disease, inflammation, or an autoimmune response, disorder or disease or an adverse symptom of an undesirable or aberrant immune response, disorder or disease, an inflammatory response, disorder or disease, inflammation, or an autoimmune response, disorder or disease; a subject that is in need of treatment for an undesirable or aberrant immune response, disorder or disease, an inflammatory response, disorder or disease, inflammation, or an autoimmune response, disorder or disease or an adverse symptom of an undesirable or aberrant immune response, disorder or disease, an inflammatory response, disorder or disease, inflammation, or an autoimmune response, disorder or disease; and a subject at risk of an undesirable or aberrant immune response, disorder or disease, an inflammatory response, disorder or disease, inflammation, or an autoimmune response, disorder or disease or an adverse symptom of an undesirable or aberrant immune response, disorder or disease, an inflammatory response, disorder or disease, inflammation, or an autoimmune response, disorder or disease.

Invention uses and methods of modulating an immune response in a subject include immune and inflammatory responses that are an anti-cancer or anti-pathogen immune response or inflammatory response.

The invention also provides methods of screening for an agent for binding of 4-1BB to a galectin. In one aspect, a method includes contacting 4-1BB with a galectin in the presence of a test agent under conditions allowing binding of 4-1BB to the galectin; and determining if the test agent modulates binding of 4-1BB to the galectin. A determination that the test agent modulates binding of 4-1BB to the galectin indicates that the test agent is an agent that modulates binding of 4-1BB with the galectin.

The invention further provides methods of identifying an agent that modulates binding of 4-1BB to a galectin. In one aspect, a method includes contacting 4-IBB with the galectin in the presence a test agent under conditions allowing binding of 4-1BB to the galectin; and determining if the test agent modulates binding of 4-1BB to the galectin. A determination that the test agent modulates binding of 4-1BB to the galectin indicates that the test agent is an agent that modulates binding of 4-1BB to the galectin.

The invention moreover provides peptide sequences that modulate binding of 4-1BB to a galectin, such as Galectin-9. In particular aspects, a peptide includes or consists of a fragment of galectin or Galectin-9 amino acid sequence that binds to a 4-1BB amino acid sequence. In additional aspects, a peptide includes or consists of a fragment of galectin or Galectin-9 amino acid sequence that binds to a Tim-3 amino acid sequence. In further aspects, a peptide includes or consists of a fragment of galectin or Galectin-9 amino acid sequence that binds to a CD44 amino acid sequence. In yet additional aspects a peptide includes or consists of a fragment of a 4-1BB amino acid sequence that binds to a galectin or Galectin-9 amino acid sequence. In still further aspects, a peptide includes or consists of a fragment of a 4-1BB amino acid sequence that binds to a 4-1BBL amino acid sequence. In still additional aspects a peptide includes or consists of a fragment of a 4-1BBL amino acid sequence that binds to a 4-1BB amino acid sequence, and includes or consists of a fragment of a Tim-3 amino acid sequence that binds to a Galectin-9 amino acid sequence.

Such peptide sequences that modulate binding of 4-1BB to a galectin, such as Galectin-9, have various lengths. In particular aspects, a peptide has a sequence length from about 10 to about 20, L- or D-amino acids, about 20 to about 50 L- or D-amino acids, about 50 to about 100 L- or D-amino acids, about 100 to about 150 L- or D-amino acids, or from about 150 to about 200 L- or D-amino acids.

### BRIEF DESCRIPTION OF THE DRAWINGS

FIGS. 1A and 1B show that 4-1BB binds to Galectin-9 as well as 4-1BBL. A) Recombinant proteins (mGalectin-1, hGalectin-9, m4-1BBL) were coated to an ELISA plate for 2 hr at 37° C., followed by washing and blocking for non-specific binding. m4-1BB.Fc, hIgG, or mBTLA.Fc were then added for 1 hr at RT again followed by washing. Anti-Fc-HRP was added for a further 1 hr at RT and color developed with OPD. Binding was determined by measurement of O.D. in standard ELISA fashion. B) Protein G beads were coated with 1 μg of m4-1BB.Fc or human Fc by incubating in 50 μL of PBS (0.02% Tween 20) at RT for 10 mins. Beads were washed, then incubated at RT for 15 mins with 1 μg of m4-1BBL or mGalectin-9 in 100 μL PBS (0.02% Tween 20). Beads were then washed and stained with PE-anti-4-1BBL or PE-anti-Galectin-9 and analyzed by flow cytometry.

FIG. 2 shows that Galectin-9 and 4-1BBL do not compete with each other for binding to 4-1BB. Protein G beads were coated with 1 μg of m4-1BB.Fc by incubating in 50 μL of PBS (0.02% Tween 20) at RT for 10 mins. Beads were washed, then incubated at RT for 15 mins with saturating amounts (5 μg) of m4-1BBL or mGalectin-9 in 100 μL PBS (0.02% Tween 20). For competition analysis, 4-1BBL or Galectin-9 pre-incubated beads were washed twice and further incubated with 1 μg of Galectin-9 or 4-1BBL in 50 μL PBS (0.02% Tween 20) respectively at RT for 15 mins. Beads were then washed and stained with APC-anti-4-1BBL and PE-anti-Galectin-9 (blue) or isotype control antibodies (red) and analyzed by flow cytometry.

FIGS. 3A and 3B show that soluble Galectin-9 binding to activated CD4 T cells is dependent on 4-1BB expression and blocked with antibodies to 4-1BB. MACS purified CD4 T cells from C57BL/6 WT or 4-1BB^{-/-} mice were activated with 3 μg/ml anti-CD3 for 2 days. A) Activated T cells were incubated with recombinant mGalectin-9 at a concentration of 1 μg/million cells in 100 μL PBS at 4° C. for 30 mins. Cells were washed, incubated with PE-anti-Galectin-9 at 4° C. for 30 mins, then washed and analyzed by flow cytometry for binding of Galectin-9. B) Activated WT T cells were preincubated with various anti-4-1BB antibodies (clone 3H3 or 1D8) at a concentration of 1 μg/million cells. Cells were washed and incubated with recombinant mGalectin-9 at a concentration of 1 μg/million cells in 100 μL PBS at 4° C. for 30 mins. Cells were washed, incubated with PE-anti-Galectin-9 at 4° C. for 30 mins, then washed and analyzed by flow cytometry for binding of Galectin-9. MFI was calculated for Galectin-9 binding.

FIGS. 4A and 4B show that Galectin-9 co-immunoprecipitates with 4-1BB. A) 4-1BB^{-/-} murine T hybridoma cells were transduced with Myc-tagged m4-1BB, and either stimulated with anti-4-1BB plus anti-CD3 for 15 min, or left unstimulated (NS). Cells were harvested and lysed in 1% NP-40 lysis buffer and 4-1BB was immunoprecipitated using anti-Myc antibody. Precipitates were assessed for 4-1BB and Gal-9 content by western blot with either anti-4-1BB or anti-Galectin-9. Mock control cells lacking 4-1BB were used as a negative control. B) T hybridoma cells transduced with Myc-tagged full length 4-1BB (FL) or a mutant form of 4-1BB lacking its entire cytoplasmic portion (ΔC) were lysed in the same buffer as A. 4-1BB was immunoprecipitated using anti-Myc antibody, followed by western blotting with either anti-4-1BB or anti-Galectin-9 antibody.

FIGS. 5A and 5B show that Galectin-9 is a binding partner for both human and murine 4-1BB, and murine 4-1BB binds to murine Galectin-9 in a carbohydrate independent manner. A) Protein G beads were coated with 5 μg of human or murine 4-1BB.Fc by incubating in 50 μL of PBS (0.02% Tween 20) at RT for 10 mins. Beads were washed, then incubated at RT for 15 mins with 5 μg of various recombinant proteins (human or mouse 4-1BBL, human or mouse Galectin-9 and human or mouse Galectin-4) in 100 μL PBS (0.02% Tween 20). Beads were washed 3× times and bound proteins were eluted and SDS-PAGE was performed. B) For elucidating the carbohydrate dependency of mouse 4-1BB binding to mouse Galectin-9, experiment was performed as in A, except that 4-1BB.Fc was initially treated with either O-Glycosidase or PNGaseF enzymes to remove O-linked or N-linked carbohydrate chains respectively.

FIG. 6 shows a surface plasmon resonance analysis of the interaction of murine Galectin-9 with murine 4-1BB. The equilibrium binding constant (K_{D}) for murine Galectin-9 interacting with murine 4-1BB.Fc immobilized on a CM-5 sensor chip was determined by SPR using a Biacore 3000. The concentration range of injected Galectin-9 was 3.9-125 nM in six serial dilutions as indicated. Data are presented as real-time graphs of response units (RU) against time and are analyzed using a model for 1:1 (Langmuir) binding reactions from the BIAeval software.

FIG. 7 shows a surface plasmon resonance analysis of the interaction of human Galectin-9 with human 4-1BB. The equilibrium binding constant (K_{D}) for human Galectin-9 interacting with human 4-1BB.Fc immobilized on a CM-5 sensor chip was determined by SPR using a Biacore 3000. The concentration range of injected Galectin-9 was 0.03125 μM-0.5 in five serial dilutions as indicated. Data are presented as real-time graphs of response units (RU) against time and are analyzed using a model for 1:1 (Langmuir) binding reactions from the BIAeval software.

FIG. 8. shows that Galectin-9 expression is required for 4-1BB signaling to promote RALDH activity in dendritic cells. CD11c+ splenic dendritic cells (DC) from WT or Galectin-9^{-/-} mice were cultured with TLR2 ligand Zymosan (2.5 μg/ml, Sigma-Aldrich) in the presence of 10 μg/ml rat IgG or agonist anti-4-1BB (clone 3H3) for 48 h. Cells were harvested and activity of retinal dehydrogenase (RALDH) was determined by ALDEFLUOR staining. Percent RALDH+ cells indicated. MFI of staining in parentheses.

FIGS. 9A and 9B show that Galectin-9 expression is required for 4-1BB signaling to promote cytokine production in CD4 and CD8 T cells. A) Purified CD4 or CD8 T cells from C57BL/6 WT or Galectin-9^{-/-} mice were activated with 1 μg/ml anti-CD3 in the presence of 10 μg/ml rat IgG or agonist anti-4-1BB (clone 3H3) for 3 days. Culture supernatants were harvested and IL-2 (for CD4 cultures) and IFN-γ (for CD8 cultures) secretion assayed by ELISA. B) Purified CD4 or CD8 T cells from C57BL/6 WT or Galectin-9^{-/-} mice were activated with 3 μg/ml of soluble anti-CD3 and 1 μg/ml of anti-CD28 for 2 days. Cells were cultured for another 16 hrs by replacing ¾ of the culture supernatant with fresh media. The activated/effector T cells were washed and restimulated with 0.5 μg/ml of soluble anti-CD3 in the presence of 10 μg/ml rat IgG or agonist anti-4-1BB (clone 3H3) for another 2 days. Culture supernatants were harvested and IL-2 (for CD4 cultures) and IFN-γ (for CD8 cultures) secretion measured.

FIGS. 10A and 10B show that anti-4-1BB mediated suppression of EAE is attenuated in the absence of Galectin-9. To induce EAE disease, 8 week old WT and Galectin-9^{-/-} mice were immunized s.c. at the base of the tail with 100 μg of MOG₃₅₋₅₅ peptide (AnaSpec) emulsified in an equal volume of CFA containing 2 mg/ml Mycobacterium Tuberculosis H37 RA (Difco). The mice also received an i.v. injection of 200 ng pertussis toxin (List Biological Laboratories) on day 0 and 2. Animals were injected i.p. with 25 μg agonist anti-4-1BB (clone 3H3) or rat IgG control antibody on day 0, 2, 4 and 8. A) Individual animals were scored daily for clinical signs of EAE using the following criteria: 0, no detectable signs of disease; 0.5, distal limp tail; 1, complete limp tail; 1.5, limp tail and hind limb weakness; 2, unilateral partial hind paralysis; 2.5, bilateral partial hind limb paralysis; 3, complete bilateral hind limb paralysis; 3.5, complete bilateral hind limb paralysis and unilateral forelimb; 4, total paralysis of fore and hind limbs. Mean clinical score was calculated by adding clinical score of individual mice and dividing by the number of mice in each group (n=5). The data are mean±SEM and are representative of three independent experiments. B) Spinal cords from EAE-induced mice were dissected, fixed in 4% PFA (Electron Microscopy Sciences), and paraffin embedded. Sections (5 μm) were stained with H&E. To ensure comparable analyses between different groups, six to eight randomly selected sections were analyzed per animal. Representative sections shown.

FIG. 11 shows that anti-4-1BB does not promote expansion of suppressive CD8β⁺CD11c⁺IFN-γ⁺ cells in Galectin-9-deficient mice during induction of EAE. WT and Galectin-9^{-/-} mice were immunized to induce EAE as in FIG. 10, and injected with control IgG or agonist anti-4-1BB also as in FIG. 10. Draining lymph nodes cells were harvested on day 15 and stimulated with 50 ng/ml PMA and 500 ng/ml ionomycin in medium containing Brefeldin A for 5 hrs. Cells were stained with PerCP-Cy5.5-anti-CD8β, FITC-anti-CD11c, and intracellular APC-IFN-γ and analyzed by flow cytometry. Plots are gated CD8β⁺ cells, analyzed for CD11c and IFN-γ. Data representative of 5 animals from each group. Percent positive in each quadrant indicated.

FIGS. 12A and 12 B show that anti-4-1BB mediated suppression of asthmatic eosinophilic lung inflammation is attenuated in the absence of Galectin-9. To induce asthmatic lung inflammation, 8 week old C57BL/6 WT or Galectin-9^{-/-} mice were immunized i.p. with 20 μg OVA (Sigma-Aldrich) adsorbed to 2 mg of aluminum hydroxide and magnesium hydroxide (Alum; Fischer Scientific International) on days 0 and 7. Mice were challenged with 20 μg OVA in 20 μl PBS given intranasally on days 14-16. Animals were injected i.p. with 200 μg agonist anti-4-1BB (clone 3H3) or control rat IgG 1 day before the first OVA immunization. Mice were sacrificed on day 18, and bronchoalveolar lavage (BAL) of the lungs was performed. Cells in the lavage fluid were counted using a hemocytometer, and then stained with APC-anti-CD11c and PE-anti-SiglecF and analyzed by flow cytometry. A) Representative plots of CD11c versus SiglecF, with eosinophils being SiglecF+CD11c−. B) Absolute number of Eosinophils (SiglecF⁺CD11c⁻) in BAL from each animal were calculated based on the percentages obtained by flow cytometry. Data represents mean±SEM of 4 animals per group.

FIG. 13 shows that anti-4-1BB does not promote expansion of suppressive CD8β⁺CD11c⁺IFN-γ⁺ cells in Galectin-9-deficient mice during induction of asthmatic lung inflammation. WT and Gal-9^{-/-} mice were immunized to induce asthmatic lung inflammation as in FIG. 12, and injected with control IgG or agonist anti-4-1BB also as in FIG. 12. On day 18, lung draining lymph node cells were stimulated with 50 ng/ml PMA and 500 ng/ml ionomycin in medium containing Brefeldin A for 5 hrs. Cells were stained with PerCP-Cy5.5-anti-CD8β and FITC-anti-CD11c, and APC-anti-IFN-γ and analyzed by flow cytometry. Plots are gated CD8β⁺ cells, analyzed for CD11c and IFN-γ. Data representative of 4 animals from each group. Percent positive in each quadrant indicated.

### DETAILED DESCRIPTION

The invention is based in part upon the binding or interaction between 4-1BB and a galectin (e.g., Galectin-9 or Gal-9), and other proteins of biological (e.g., immunological) relevance. The binding or interaction can be modulated as set forth herein in order to modulate effector functions and/or activities of such binding or interaction, including immune responses. Accordingly, the invention provides methods for modulating an immune response as well as agents (e.g., peptides, etc.) that modulate the binding or interaction between 4-1BB and a galectin (e.g., Galectin-9), and other proteins of biological (e.g., immunological) relevance and are useful for modulating an immune response.

In one embodiment, a method for modulating an immune response includes contacting 4-1BB or a galectin (e.g., Galectin-9 or Gal-9) with an agent that modulates binding between 4-1BB and a galectin (e.g., Galectin-9 or Gal-9). In another embodiment, a method for modulating an immune response includes contacting an agent that binds to 4-1BB, binds to a galectin, binds to 4-1BB ligand (4-1BBL), binds to Tim-3 or binds to CD44 with 4-1BB, 4-1BBL, Tim-3 or CD44 to modulate binding between 4-1BB and a galectin, thereby modulating an immune response. In particular aspects, an agent is administered or delivered to a subject such as a mammalian (e.g., human) subject.

Members of the TNFR superfamily control diverse aspects of many immune responses and accumulating evidence has established the importance of TNF/TNFR superfamily (SF) interactions in regulating multiple cell types, including T cells, B cells, dendritic cells, NKT cells, and NK cells (1-3). It has generally been assumed that these activities are driven by binding of these molecules to their cognate ligands in the TNF superfamily. However, although the recognized ligand for 4-1BB, a member of the TNF super-family named 4-1BBL or TNFSF9, appears to explain some of the functional activities associated with 4-1BB, data disclosed herein in a number of systems indicates that 4-1BB has an immunostimulatory role and an immunosuppressive role that is explained by its ability to bind Galectin-9.

The data herein identifies a never before characterized example of cross-talk of a TNFR family molecule with a molecule in a distinct family, namely 4-1BB binding to Galectin-9. 4-1BB (also referred to as CD137 or TNFRSF9) is a 30 kD type I transmembrane glycoprotein, containing 4 cysteine rich domains (CRD) which are the basic organizational and binding domains common to the TNFR family. Although not wishing to be bound by any theory, 4-1BB is believed to function in a trimeric arrangement of 3 monomers, induced when bound to 4-1BBL (TNFSF9), its ligand in the TNF family (4-6).

Without being limited to any particular theory, binding between 4-1BB and Galectin-9 described herein appears to account for some of the effects on immune response previously attributed to 4-1BBL. The discovery disclosed herein that murine 4-1BB can bind to an unrelated ligand, namely, Galectin-9, is likely to explain various biologically relevant activities of 4-1BB. Furthermore, understanding that Galectin-9 binds to 4-1BB also provides a target for modulating the functions and activities of 4-1BB and/or Galectin-9, as well as other proteins that bind to 4-1BB or Galectin-9.

Galectin-9 is a member of a family of animal lectins that has also been described to have both pro- and anti-inflammatory activities. Galectin-9 is thought largely to recognize carbohydrate side chains of protein receptors. Galectin-9 (7, 8) is a bivalent tandem repeat-type member of the galectin family, similar to Gal-4, -6, -8, and -12, each characterized by two domains connected by a linker. The canonical binding of galectins has been reported to be between carbohydrate recognition regions and oligosaccharide side chains of proteins (9-13).

Positive and negative functions have been described for most of the galectin family members. The function most likely depends on where and when galectins are expressed, and the proteins with which they interact (14). Binding partners for Gal-9 are Tim-3 (15), a member of the immunoglobulin superfamily, and CD44 (16), a cell surface glycoprotein known to bind hyaluronic acid. Both Tim-3 and CD44 interact with Gal-9 in a manner dependent on carbohydrate recognition.

As disclosed herein, murine and human Galectin-9 bind to both murine and human 4-1BB. Without being limited to any particular theory, functions or activities of Galectin-9 could be mediated either independently by 4-1BB, or in co-operation with other binding partners (e.g., Tim-3, CD44, etc.) depending on relative expression. Similarly, without being limited to any particular theory, functions or activities of 4-1BB could be mediated either by binding or interaction with Galectin-9 or 4-1BBL, or both molecules, again dependent on relative expression.

Like 4-1BB (4-6), both stimulatory and suppressive activities have also been attributed to Galectin-9 (15-24). Thus agents that target, or include or consist of, Galectin-9 (e.g., antibodies that bind to Galectin-9, Galectin-9 amino acid sequences, or Galectin-9 fusion proteins, such as Galectin-9.Fc) or its ligands may result in both positive and negative activities on immune responses and immune disease.

Also disclosed herein is that the functional activity of 4-1BB in several settings is dependent on Gal-9. 4-1BB and its ligands can promote activities of T cells, NK cells and dendritic cells, but conversely they can also result in suppressive activities that promote T cell tolerance and limit autoreactivity and inflammation. Thus agents that target, or include or consist of, 4-1BB (e.g., antibodies that bind to 4-1BB, 4-1BB amino acid sequences, or 4-1BB fusion proteins, such as 4-1BB.Fc) or its ligands result in both positive and negative activities on immune responses and immune disease. For example, anti-4-1BB promotes tumor immunity and enhances T cell priming against viruses, but also results in suppression of autoimmunity in mouse models of lupus, MS, RA, and GVHD (3-6).

Thus, in accordance with the invention, binding between 4-1BB and a galectin, such as Galectin-9, may be modulated to either enhance or suppress an immune response. Such modulation provides therapeutically relevant uses and methods for treating a number of disorders and disease conditions such as those associated with or caused by immune responses.

Accordingly, in various embodiments, the invention provides methods and uses that modulate (alter) one or more immune responses. In one embodiment, a method or use includes contacting 4-1BB or a galectin with an agent that modulates binding of 4-1BB to the galectin, thereby modulating an immune response. In another embodiment, a method or use includes administering to a subject an agent that modulates binding of 4-1BB to the galectin, thereby modulating an immune response in the subject. In all embodiments of the invention, an agent can modulate binding of 4-1BB to the galectin, or an agent can modulate binding between 4-1BB and the galectin, or an agent can modulate binding between other molecules with which 4-1BB and the galectin may bind. Accordingly, an agent can modulate binding between any of 4-1BB, and/or 4-1BBL, and/or a galectin (e.g., Galectin-9), and/or Tim-3, and/or CD44.

In one embodiment, a method comprises decreasing, reducing, inhibiting, suppressing or disrupting binding of 4-1BB to a galectin. In other embodiments, a method comprises increasing, enhancing, stimulating, or promoting binding of 4-1BB to a galectin. In some embodiments, decreasing, reducing, inhibiting, suppressing or disrupting binding of 4-1BB to a galectin may decrease, reduce, inhibit, suppress, limit or control an immune response, disorder or disease, an inflammatory response, disorder or disease, inflammation, or an autoimmune response, disorder or disease. In other embodiments, decreasing, reducing, inhibiting, suppressing or disrupting binding of 4-1BB to a galectin may increase, stimulate, enhance, promote, induce or activate an immune response, inflammatory response or inflammation. In still further embodiments, increasing, enhancing, stimulating, or promoting binding of 4-1BB to a galectin may decrease, reduce, inhibit, suppress, limit or control an immune response, disorder or disease, an inflammatory response, disorder or disease, inflammation, or an autoimmune response, disorder or disease. In other embodiments, increasing, enhancing, stimulating, or promoting binding of 4-1BB to a galectin may increase, stimulate, enhance, promote, induce or activate an immune response, inflammatory response or inflammation.

In additional particular embodiments, there are provided uses and methods of modulating binding of 4-1BB to Galectin-9, comprising providing an agent that decreases, reduces, inhibits, suppresses or disrupts binding of 4-1BB to the galectin to decrease, reduce, inhibit, suppress, limit or control an immune response, disorder or disease, an inflammatory response, disorder or disease, inflammation, or an autoimmune response, disorder or disease. In another embodiment, there is provided a method of modulating binding of 4-1BB to Galectin-9 comprising providing an agent that increases, enhances, stimulates, or promotes binding of 4-1BB to Galectin-9 to increase, stimulate, enhance, promote, induce or activate an immune response, inflammatory response or inflammation, for example an anti-cancer or anti-pathogen immune response or inflammatory response.

As used herein, the term “modulate” or “modulating” an immune response or binding or interaction includes but is not limited to modifying, altering or affecting an immune response, such as decreasing, reducing, inhibiting, slowing, suppressing, antagonizing, or limiting an immune response, or stimulating, increasing, hastening, agonizing or enhancing an immune response. Such modulation can result from a direct or indirect interaction between an agent and any of 4-1BB, 4-1BBL, a galectin (e.g., Galectin-9), Tim-3, or CD44.

As used herein, an “agent” that modulates binding between 4-1BB and a galectin (e.g., Galectin-9) includes any agent that can modulate the binding of 4-1BB with a galectin. Such agents therefore include agents that bind to any of 4-1BB, 4-1BBL, a galectin (e.g., Galectin-9), Tim-3, or CD44.

As used herein, the term “binding” refers to any interaction between two molecules, whether direct or indirect or whether functional or physical. Thus, the term binding may refer to a physical interaction at the molecular level or functional interaction that need not require physical interaction or binding. Thus, for example an agent that inhibits binding between 4-1BB and Galectin-9 partially or completely inhibits, decreases or reduces a physical interaction or a functional interaction between 4-1BB and Galectin-9. Inhibition of binding can be due to steric hinderance, occupation, blocking or modification or alteration of the site of physical or functional interaction, or alteration of a modification or another factor (e.g., Tim-3 or CD44) that participates in binding between 4-1BB and Galectin-9. Accordingly, agents that function as inhibitors of binding between 4-1BB and Galectin-9 can act directly or indirectly upon 4-1BB and/or Galectin-9. For example, a peptide comprising a 4-1BB binding region of Galectin-9 can be an inhibitor that binds to 4-1BB, thereby inhibiting binding between 4-1BB and Galectin-9. In another non-limiting example, a peptide comprising a Galectin-9 binding region of 4-1BB can be an inhibitor that binds to Galectin-9, thereby inhibiting binding between Galectin-9 and 4-1BB.

Binding and interaction as used herein includes both cis and trans binding or interaction. As used herein, a “cis” interaction or binding refers to interaction/binding of two entities (e.g., proteins) expressed on the same cell. A “trans” interaction or binding refers to interaction/binding between proteins expressed on distinct cells (i.e., two different cells). Such cis and trans interactions between two entities can involve a direct interaction/binding. Alternatively, such cis and trans interactions between two entities can also be mediated by an intermediary molecule and need not involve direct interaction/binding between the two entities. For example, a “trans” interaction between two cells can occur when soluble Galectin-9 binds to 4-1BB on one cell to link 4-1BB to Tim-3 or CD44 expressed on another cell. Thus, a galectin such as Galectin-9 (or another intermediary) can function as an intermediary that mediates the interaction/binding of 4-1BB to Tim-3 or CD44, for example.

Galectins can be expressed as soluble molecules, and are also found on the membrane of immune cells. Galectins may therefore participate in or mediate both trans interactions (binding proteins on distinct cells) as well as cis interactions (binding proteins on the same cell). Thus in certain embodiments of the invention, binding of 4-1BB to a galectin that is to be modulated is or mediates a cis interaction. In other embodiments of the invention, the binding of 4-1BB to a galectin that is to be modulated is or meditates trans interaction.

The term “contacting” means direct or indirect binding or interaction between two or more entities (e.g., between an agent and a target, such as 4-1BB, 4-1BBL, a galectin (e.g., Galectin-9), Tim-3, or CD44). A particular example of direct interaction is physical binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration, or delivery.

Agents include agents that decrease, reduce, inhibit, suppress or disrupt binding of 4-1BB to a galectin. Agents also include agents that increase, enhance, stimulate, or promote binding of 4-1BB to a galectin. Furthermore, agents include antagonists and agonists of 4-1BB, 4-1BBL, a galectin (e.g., Galectin-9), Tim-3, or CD44 function or activity, i.e., agents that decrease, reduce, inhibit, suppress or disrupt a function or activity of 4-1BB, 4-1BBL, a galectin (e.g., Galectin-9), Tim-3, or CD44; or increase, enhance, stimulate, or promote a function or activity of 4-1BB, 4-1BBL, a galectin (e.g., Galectin-9), Tim-3, or CD44.

Non-limiting particular examples of agents include amino acid sequences, such as antibodies, proteins, peptides, and polypeptides, including fusion polypeptides and chimeric polypeptides. Non-limiting examples of agents also include nucleic acid sequences or polynucleotides/polynucleosides. Further non-limiting examples of agents include small molecules. All of such agents can bind to any of 4-1BB, 4-1BBL, a galectin (e.g., Galectin-9), Tim-3, or CD44, thereby modulating (altering or affecting) binding between 4-1BB and a galectin, and in turn modulating an immune response.

As disclosed herein, agents include antibodies. Such antibodies can modulate binding of 4-1BB to a galectin, and include, for example, antibodies and antibody subsequences (fragments) that bind to any one of 4-1BB, a galectin such as Gal-9, 4-1BB ligand, Tim-3 or CD44.

As used herein, an “antibody” refers to a protein that binds to other molecules (antigens) via heavy and light chain variable domains, V_{H} and V_{L}, respectively. Antibodies include full-length antibodies that include two heavy and two light chain sequences. Antibodies can have kappa or lambda light chain sequences, either full length as in naturally occurring antibodies, mixtures thereof (i.e., fusions of kappa and lambda chain sequences), and subsequences/fragments thereof. Naturally occurring antibody molecules contain two kappa or two lambda light chains.

The term “bind,” or “binding,” when used in reference to an antibody or subsequence thereof, means that the antibody or subsequence thereof interacts at the molecular level with an epitope (antigenic determinant) present on the target. Thus, an antibody binds to all or a part of a target (e.g., any of 4-1BB, 4-1BBL, a galectin (e.g., Galectin-9), Tim-3, or CD44) sequence. Specific binding is that which is selective for the target (e.g., for 4-1BB, 4-1BBL, a galectin (e.g., Galectin-9), Tim-3, or CD44). Specific and selective binding can be distinguished from non-specific binding using assays known in the art (e.g., competition binding, immunoprecipitation, ELISA, flow cytometry, Western blotting).

Antibodies include polyclonal and monoclonal antibodies. The term “monoclonal,” when used in reference to an antibody refers to an antibody that is based upon, obtained from or derived from a single clone, including any eukaryotic, prokaryotic, or phage clone. A “monoclonal” antibody is therefore defined herein structurally, and not the method by which it is produced.

Antibodies can belong to any antibody class, IgM, IgG, IgE, IgA, IgD, or subclass. Exemplary subclasses for IgG are IgG₁, IgG₂, IgG₃ and IgG₄.

Antibodies include antibody subsequences and fragments. Such subsequences and fragments can have the binding affinity as the full length antibody, the binding specificity as the full length antibody, or one or more activities or functions of as a full length antibody, e.g., a function or activity of 4-1BB, 4-1BBL, a galectin (e.g., Galectin-9), Tim-3, or CD44 binding antibody. Exemplary antibody subsequences and fragments include an Fab, Fab′, F(ab′)₂, Fv, Fd, single-chain Fv (scFv), disulfide-linked Fvs (sdFv), V_{L}, V_{H}, Camel Ig, V-NAR, VHH, trispecific (Fab₃), bispecific (Fab₂), diabody ((V_{L}-V_{H})₂ or (V_{H}-V_{L})₂), triabody (trivalent), tetrabody (tetravalent), minibody ((scF_{V}-C_{H}3)₂), bispecific single-chain Fv (Bis-scFv), IgGdeltaC_{H}2, scFv-Fc or (scFv)₂-Fc.

Antibody subsequences and fragments can be combined. For example, a V_{L} or V_{H} subsequences can be joined by a linker sequence thereby forming a V_{L}-V_{H} chimera. A combination of single-chain Fvs (scFv) subsequences can be joined by a linker sequence thereby forming a scFv-scFv chimera. Antibody subsequences and fragments include single-chain antibodies or variable region(s) alone or in combination with all or a portion of other subsequences.

Antibody subsequences and fragments can be prepared by proteolytic hydrolysis of the antibody, for example, by pepsin or papain digestion of whole antibodies. Antibody subsequences and fragments produced by enzymatic cleavage with pepsin provide a 5S fragment denoted F(ab′)₂. This fragment can be further cleaved using a thiol reducing agent to produce 3.5S Fab′ monovalent fragments. Alternatively, an enzymatic cleavage using pepsin produces two monovalent Fab′ fragments and the Fc fragment directly (see, e.g., U.S. Pat. Nos. 4,036,945 and 4,331,647; and Edelman et al., Methods Enymol. 1:422 (1967)). Other methods of cleaving antibodies, such as separation of heavy chains to form monovalent light-heavy chain fragments, further cleavage of fragments, or other enzymatic or chemical may also be used.

Epitopes typically are short amino acid sequences, e.g. about five to 15 amino acids in length. Epitopes can be contiguous or non-contiguous. A non-contiguous amino acid sequence epitope forms due to protein folding. For example, an epitope can include a non-contiguous amino acid sequence, such as a 5 amino acid sequence and an 8 amino acid sequence, which are not contiguous with each other, but form an epitope due to protein folding. Techniques for identifying epitopes are known to the skilled artisan and include screening overlapping oligopeptides for binding to antibody (for example, U.S. Pat. No. 4,708,871), phage display peptide library kits, which are commercially available for epitope mapping (New England BioLabs). Epitopes may also be identified by inference when epitope length peptide sequences are used to immunize animals from which antibodies that bind to the peptide sequence are obtained and can be predicted using computer programs, such as BEPITOPE (Odorico et al., J. Mol. Recognit. 16:20 (2003)).

Antibodies and fragments thereof include mammalian, primatized, humanized, fully human antibodies and chimeras. A mammalian antibody is an antibody produced by a mammal, transgenic or non-transgenic, or a non-mammalian organism engineered to produce a mammalian antibody, such as a non-mammalian cell (bacteria, yeast, insect cell), animal or plant.

The term “human” when used in reference to an antibody, means that the amino acid sequence of the antibody is fully human, i.e., human heavy and human light chain variable and human constant regions. Thus, all of the amino acids are human or exist in a human antibody. An antibody that is non-human may be made fully human by substituting the non-human amino acid residues with amino acid residues that exist in a human antibody. Amino acid residues present in human antibodies, CDR region maps and human antibody consensus residues are known in the art (see, e.g., Kabat, Sequences of Proteins of Immunological Interest, 4^{th} Ed. US Department of Health and Human Services. Public Health Service (1987); Chothia and Lesk (1987). A consensus sequence of human V_{H} subgroup III, based on a survey of 22 known human V_{H} III sequences, and a consensus sequence of human V_{L} kappa-chain subgroup I, based on a survey of 30 known human kappa I sequences is described in Padlan Mol. Immunol. 31:169 (1994); and Padlan Mol. Immunol. 28:489 (1991). Human antibodies therefore include antibodies in which one or more human or non-human amino acid residues have been substituted with one or more amino acids present in any other human antibody.

The term “humanized” when used in reference to an antibody, means that the amino acid sequence of the antibody has non-human amino acid residues (e.g., mouse, rat, goat, rabbit, etc.) of one or more complementarity determining regions (CDRs) that specifically bind to the desired antigen in an acceptor human immunoglobulin molecule, and one or more human amino acid residues in the Fv framework region (FR), which are amino acid residues that flank the CDRs. Such antibodies typically have reduced immunogenicity and therefore a longer half-life in humans as compared to the non-human parent antibody from which one or more CDRs were obtained or are based upon.

“Primatized” antibodies are “humanized,” except that the acceptor human immunoglobulin molecule and framework region amino acid residues may be any primate amino acid residue (e.g., ape, gibbon, gorilla, chimpanzees orangutan, macaque), in addition to any human residue. Human FR residues of the immunoglobulin can be replaced with corresponding non-human residues. Residues in the CDR or human framework regions can therefore be substituted with a corresponding residue from the non-human CDR or framework region donor antibody to alter, generally to improve, antigen affinity or specificity, for example. A humanized antibody may include residues, which are found neither in the human antibody nor in the donor CDR or framework sequences. For example, a FR substitution at a particular position that is not found in a human antibody or the donor non-human antibody may be predicted to improve binding affinity or specificity human antibody at that position. Antibody framework and CDR substitutions based upon molecular modeling are well known in the art, e.g., by modeling of the interactions of the CDR and framework residues to identify framework residues important for antigen binding and sequence comparison to identify unusual framework residues at particular positions (see, e.g., U.S. Pat. No. 5,585,089; and Riechmann et al., Nature 332:323 (1988)).

The term “chimera” or “chimeric” and grammatical variations thereof, when used in reference to an antibody or subsequence thereof, means that the amino acid sequence of the antibody contains one or more portions that are derived from, obtained or isolated from, or based upon two or more different species. For example, a portion of the antibody may be human (e.g., a constant region) and another portion of the antibody may be non-human (e.g., a murine heavy or murine light chain variable region). Thus, an example of a chimeric antibody is an antibody in which different portions of the antibody are of different species origins. Unlike a humanized or primatized antibody, a chimeric antibody can have the different species sequences in any region of the antibody.

Methods of producing polyclonal and monoclonal antibodies are known in the art. For example, 4-1BB, 4-1BBL, a galectin (e.g., Galectin-9), Tim-3, or CD44, or a subsequence thereof, or an immunogenic fragment thereof, optionally conjugated to a carrier such as keyhole limpet hemocyanin (KLH) or ovalbumin (e.g., BSA), or mixed with an adjuvant such as Freund's complete or incomplete adjuvant may be used to immunize an animal. Using conventional hybridoma technology, splenocytes from immunized animals that respond to 4-1BB, 4-1BBL, a galectin (e.g., Galectin-9), Tim-3, or CD44, or a subsequence thereof, or an immunogenic fragment thereof can be isolated and fused with myeloma cells. Monoclonal antibodies produced by the hybridomas can be screened for reactivity with 4-1BB, 4-1BBL, a galectin (e.g., Galectin-9), Tim-3, or CD44, or a subsequence thereof, or an immunogenic fragment thereof.

Animals that may be immunized include mice, rats, rabbits, goats, sheep, cows or steer, guinea pigs or primates. Initial and any optional subsequent immunization may be through intravenous, intraperitoneal, intramuscular, or subcutaneous routes. Subsequent immunizations may be at the same or at different concentrations of 4-1BB, 4-1BBL, a galectin (e.g., Galectin-9), Tim-3, or CD44, or a subsequence thereof, or an immunogenic fragment thereof, preparation, and may be at regular or irregular intervals.

Antibodies can also be generated using other techniques including hybridoma, recombinant, and phage display technologies, or a combination thereof (see U.S. Pat. Nos. 4,902,614, 4,543,439, and 4,411,993; see, also Monoclonal Antibodies, Hybridomas: A New Dimension in Biological Analyses, Plenum Press, Kennett, McKearn, and Bechtol (eds.), 1980, and Harlow et al., Antibodies: A Laboratory Manual, Cold Spring Harbor Laboratory Press, 2nd ed. 1988).

Antibodies can be humanized using a variety of techniques known in the art including, for example, CDR-grafting (EP 239,400; WO91/09967; U.S. Pat. Nos. 5,225,539; 5,530,101; and 5,585,089), veneering or resurfacing (EP 592,106; EP 519,596; Padlan, Molecular Immunol. 28:489 (1991); Studnicka et al., Protein Engineering 7:805 (1994); Roguska. et al., Proc. Nat'l. Acad. Sci. USA 91:969 (1994)), and chain shuffling (U.S. Pat. No. 5,565,332). Human consensus sequences (Padlan, Mol. Immunol. 31:169 (1994); and Padlan, Mol. Immunol. 28:489 (1991)) have previously used to produce humanized antibodies (Carter et al., Proc. Natl. Acad. Sci. USA 89:4285 (1992); and Presta et al., J. Immunol. 151:2623 (1993)).

Antibodies and subsequences thereof include those that bind to a sequence region of a protein that binds to another protein. In particular embodiments, an antibody binds to a sequence region of 4-1BB that binds to a galectin or 4-1BBL, an antibody binds to a sequence region of a galectin (e.g., Galectin-9) that binds to 4-1BB, Tim-3 or CD44, an antibody binds to a sequence region of 4-1BB ligand (4-1BBL) that binds to 4-1BB, an antibody binds to a sequence region of Tim-3 that binds to Galectin-9, or an antibody binds to a sequence region of CD44 sequence that binds to Galectin-9.

More particularly, an antibody or subsequence thereof includes those that bind a 4-1BB peptide cysteine rich domain (CRD), such as (CRD) 2 and/or CRD 3 of 4-1BB; or a 4-1BB peptide sequence: PCPPNSFSSAGGQRTCDICRQCKGVFRTRKECSSTSNAECDCTPGFHCLGAGCSMCEQDCKQGQELTKKGCKDC, or a subsequence thereof; or a 4-1BB peptide sequence: LQDPCSNCPAGTFCDNNRNQIC, PCPPNSFSSAGGQRTCDICRQCKGVFRTRKECSSTSNAEC, DCTPGFHCLGAGCSMCEQDCKQGQELTKKGCK, or DCCFGTFNDQKRGICRPWTNCSLDGKSVLVNGTKERDVVCG, or a subsequence thereof; or an extracellular domain sequence of human 4-1BB, from about amino acid residues 1-163 of human 4-1BB, or a subsequence thereof.

More particularly, an antibody or subsequence thereof includes those that bind a 4-1BBL peptide or fragment, from about residues 49-254 of 4-1BBL, 115 to 227 of 4-1BBL, from about 104-309 of 4-1BBL, 105-309 of 4-1BBL, or 106-309 of 4-1BBL, or a subsequence thereof.

More particularly, an antibody or subsequence thereof includes those that bind a galectin or Galectin-9 peptide or fragment having a carbohydrate binding/recognition domain (CBD) of galectin or Galectin-9, or a subsequence of a carbohydrate binding/recognition domain (CBD) of galectin or Galectin-9. In particular examples, an antibody or subsequence thereof binds to a Galectin-9 peptide or fragment of CBD 1: PFSGTIQGGLQDGLQITVNGTVLSSSGTRFAVNFQTGFSGNDIAFHFNPRFEDGG YVVCNTRQNGSWGPEERKTHMPFQKGMPFDLCFLVQS SDFKVMVNGILFVQYFHRVPFHRVDTI SVNGSVQLSYIS (residues from 16-146), or a subsequence thereof; or binds to CBD2: FITTILGGLYPSKSILLSGTVLPSAQRFHINLCSGNHIAFHLNPRFDENAVVRNTQIDNSWGSE ERSLPRKMPFVRGQSFSVWILCEAHCLKVAVDGQHLFEYYHRLRNLPTINRLEVGGDIQLTHVQ (residues from 227-354), or a subsequence thereof; or binds to a Galectin-9 peptide or fragment of: PFSGTIQGGLQDGLQITVNGTVLSSSGTRFAVNFQTGFSGNDIAFHFNPRFEDGGYVVCNTRQNG, SWGPEERKTHMPFQKGMPFDLCFLVQSSDFKVMVNGILFVQYFHRVPFHRVDTISVNGSVQLSYIS, or a subsequence thereof; or FITTILGGLYPSKSILLSGTVLPSAQRFHINLCSGNHIAFHLNPRFDENAVVRNTQIDNSWGSE ERSLPRKMPFVRGQSFSVWILCEAHCLVAVDGQHLFEYYHRLRNLPTINRLEVGGDIQLTHVQ, or a subsequence thereof; or an N-terminal sequence portion of Galectin-9 from residues 50-58, 77-80, and/or 122-131 or a subsequence thereof; or a C-terminal sequence portion of Galectin-9 from residues 290-295, 310-314, and/or 326-336, or a subsequence thereof; or binds to a contiguous Gal-9 sequence comprising arginine at position 65.

More particularly, an antibody or subsequence thereof includes those that bind to all or a portion of human Tim-3, from residues 30-128.

Non-limiting particular examples of antibodies and subsequences thereof that bind to 4-1BB include PF-05082566 (Pfizer), 1D8, 3E1 or BMS-663513 (Bristol Myers Squibb), 4B4 (BioLegend 309809), H4-1BB-M127 (BD Pharmingen 552532), BBK2 (Thermo Fisher MS621PABX), 145501 (Leinco Technologies B591), and antibody produced by cell line deposited as ATCC No. HB-11248 (U.S. Pat. No. 6,974,863). Non-limiting particular examples of antibodies and subsequences thereof that bind to 4-1BBL include 5F4 (Biolegend 311503) and C65-485 (BD Pharmingen 559446). Non-limiting particular examples of antibodies and subsequences thereof that bind to Galectin-9 include 9M1-3 (Biolegend 348905), ECA42 (MBL D193-3) and 9S2-1 (Biolegend 650701). Non-limiting particular examples of antibodies and subsequences thereof that bind to CD44 include BJ18 (United States Biological C2398-02D), IM7 (Stem Cell Technologies 01465), 515 BD (Pharmingen 550990), G44-26 BD (Pharmingen 561858c), 2F10 (R&D Systems FAB3660F), 2C5 (R&D Systems BBA10) and 3G5 (R&D Systems BBA11). Non-limiting particular examples of antibodies and subsequences thereof that bind to Tim-3 include F38-2E2 (eBioscience 17-3109-41) and 344823 (R&D Systems FAB2365P).

Although typically molecules such as affibodies, aptamers, avimers and nanobodies are not strictly considered antibodies, such molecules have conserved scaffold structures, and can also be engineered to bind to targets such as 4-1BB, 4-1BBL, a galectin (e.g., Galectin-9), Tim-3, or CD44. Accordingly, such alternative scaffold structures, including but not limited to affibodies, aptamers, avimers and nanobodies, are included within the meaning of the term agent as used herein.

Human antibodies can be produced by immunizing human transchromosomic KM Mice™ (WO 02/43478) or HAC mice (WO 02/092812). KM Mice™ and HAC mice express human immunoglobulin genes. Using conventional hybridoma technology, splenocytes from immunized mice that were high responders to the antigen can be isolated and fused with myeloma cells. A monoclonal antibody can be obtained that binds to the antigen. An overview of the technology for producing human antibodies is described in Lonberg and Huszar (Int. Rev. Immunol. 13:65 (1995)). Transgenic animals with one or more human immunoglobulin genes (kappa or lambda) that do not express endogenous immunoglobulins are described, for example in, U.S. Pat. No. 5,939,598. Additional methods for producing human polyclonal antibodies and human monoclonal antibodies are described (see, e.g., Kuroiwa et al., Nat. Biotechnol. 20:889 (2002); WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598).

Methods for producing chimeric antibodies are known (e.g., Morrison, Science 229:1202 (1985); Oi et al., BioTechniques 4:214 (1986); Gillies et al., J. Immunol. Methods 125:191 (1989); and U.S. Pat. Nos. 5,807,715; 4,816,567; and 4,816,397). Chimeric antibodies in which a variable domain from an antibody of one species is substituted for the variable domain of another species are described, for example, in Munro, Nature 312:597 (1984); Neuberger et al., Nature 312:604 (1984); Sharon et al., Nature 309:364 (1984); Morrison et al., Proc. Nat'l. Acad. Sci. USA 81:6851 (1984); Boulianne et al., Nature 312:643 (1984); Capon et al., Nature 337:525 (1989); and Traunecker et al., Nature 339:68 (1989).

Suitable techniques that additionally may be employed in antibody methods include affinity purification, non-denaturing gel purification, HPLC or RP-HPLC, size exclusion, purification on protein A column, or any combination of these techniques. The antibody isotype can be determined using an ELISA assay, for example, a human Ig can be identified using mouse Ig-absorbed anti-human Ig.

As disclosed herein, agents also include proteins, peptides, and polypeptides, including fusion polypeptides and chimeric polypeptides. In various embodiments, an agent that modulates binding of 4-1BB with the galectin is a peptide or fragment of a 4-1BB, a peptide or fragment of a galectin such as Galectin-9, a peptide or fragment of a 4-1BB ligand, a peptide or fragment of a Tim-3 or a peptide or fragment of a CD44 polypeptide. Such sequences can be derived from or modelled after 4-1BB, galectin, 4-1BB ligand, Tim-3 or CD44 polypeptide sequences. The 4-1BB, galectin, 4-1BB ligand, Tim-3 or CD44 polypeptide and nucleic acid sequences include mammalian sequences, such as human, gorilla, chimpanzee, orangutan, or macaque, rodent (e.g., murine) sequences. In some embodiments, a peptide or fragment is mammalian. In particular embodiments, a peptide or fragment is human.

As used herein, a “polypeptide,” or “protein” or “peptide” refers to two, or more, amino acids linked by an amide or equivalent bond. A polypeptide can also be referred to herein, inter alia, as a protein, or an amino acid sequence, or simply a sequence. Polypeptides of the invention include L- and D-isomers, and combinations of L- and D-isomers. Polypeptides can form intra or intermolecular disulfide bonds. Polypeptides can also form higher order structures, such as multimers or oligomers, with the same or different polypeptide, or other molecules. The polypeptides can include modifications typically associated with post-translational processing of proteins, for example, cyclization (e.g., disulfide bond), phosphorylation, glycosylation, carboxylation, ubiquitination, myristylation, acetylation (N-terminal), amidation (C-terminal), or lipidation. Polypeptides described herein further include compounds having amino acid structural and functional analogues, for example, peptidomimetics having synthetic or non-natural amino acids or amino acid analogues, so long as the mimetic has one or more functions or activities of a native polypeptide set forth herein. Non-natural and non-amide chemical bonds, and other coupling means can also be included, for example, glutaraldehyde, N-hydroxysuccinimide esters, bifunctional maleimides, or N,N′-dicyclohexylcarbodiimide (DCC). Non-amide bonds can include, for example, ketomethylene aminomethylene, olefin, ether, thioether and the like (see, e.g., Spatola (1983) in Chemistry and Biochemistry of Amino Acids, Peptides and Proteins, Vol. 7, pp 267-357, “Peptide and Backbone Modifications,” Marcel Decker, NY).

Non-limiting examples of 4-1BB, a galectin such as Gal-9, 4-1BB ligand, Tim-3 and CD44 polypeptide sequences that can be targeted with agents according to the invention, or are themselves agents or can be used as agents in accordance with the invention methods and uses are as follows:

```
Human 4-1BB (255 amino acids) with cysteine rich domains (CRD) 1,
2, 3 and 4 underlined (D1, D2, D3 & D4):
                10 20 30 40 50 60
MGNSCYNIVA TLLLVLNFER TRSLQDPCSN CPAGTFCDNN RNQICSPCPP NSFSSAGGQR
                                                                      D1 D2
                70 80 90 100 110 120
TCDICRQCKG VFRTRKECSS TSNAECDCTP GFHCLGAGCS MCEQDCKQGQ ELTKKGCKDC
                                                                            D3
              130 140 150 160 170 180
CFGTFNDQKR GICRPWTNCS LDGKSVLVNG TKERDVVCGP SPADLSPGAS SVTPPAPARE
                              D4
              190 200 210 220 230 240
PGHSPQIISF FLALTSTALL FLLFFLTLRF SVVKRGRKKL LYIFKQPFMR PVQTTQEEDG
              250
CSCRFPEEEE GGCEL
Human Galectin-9 (355 amino acids) with carbohydrate binding/re-
cognition domains 1 and 2 underlined (CBD):
                10 20 30 40 50 60
MAFSGSQAPY LSPAVPFSGT IQGGLQDGLQ ITVNGTVLSS SGTRFAVNFQ TGFSGNDIAF
                70 80 90 100 110 120
HFNPRFEDGG YVVCNTRQNG SWGPEERKTH MPFQKGMPFD LCFLVQSSDF KVMVNGILFV
                                                            CBD1
              130 140 150 160 170 180
QYFHRVPFHR VDTISVNGSV QLSYISFQNP RTVPVQPAFS TVPFSQPVCF PPRPRGRRQK
              190 200 210 220 230 240
PPGVWPANPA PITQTVIHTV QSAPGQMFST PAIPPMMYPH PAYPMPFITT ILGGLYPSKS
              250 260 270 280 290 300
ILLSGTVLPS AQRFHINLCS GNHIAFHLNP RFDENAVVRN TQIDNSWGSE ERSLPRKMPF
                                                              CBD2
              310 320 330 340 350
VRGQSFSVWI LCEAHCLKVA VDGQHLFEYY HRLRNLPTIN RLEVGGDIQL THVQT
```

Individual domains of 4-1BB and Galectin-9, each of which may be involved in binding:

Cysteine rich domain 1, 2, 3 & 4 (D1, D2, D3 & D4) of Human 4-1BB:
LQDPCSNCPAGTFCDNNRNQIC
-D1 (aa 24-45)
PCPPNSFSSAGGQRTCDICRQCKGVFRTRKECSSTSNAEC
-D2 (aa 47-86)
DCTPGFHCLGAGCSMCEQDCKQGQELTKKGCK
-D3 (aa 87-118)
DCCFGTFNDQKRGICRPWTNCSLDGKSVLVNGTKERDVVCG
-D4 (aa 119-159)
Carbohydrate binding/recognition domain (CBD) 1 and 2 of Human Galectin-9:
PFSGTIQGGLQDGLQITVNGTVLSSSGTRFAVNFQTGFSGNDIAFHFNPRFEDGG
YVVCNTRQNGSWGPEERKTHMPFQKGMPFDLCFLVQSSDFKVMVNGILFVQYFHRVPFHRVDTIS
VNGSVQLSYIS
-N terminal CRD (aa 16-146)
FITTILGGLYPSKSILLSGTVLPSAQRFHINLCSGNHIAFHLNPRFDENAVVRNTQIDNSWGSEE
RSLPRKMPFVRGQSFSVWILCEAHCLKVAVDGQHLFEYYHRLRNLPTINRLEVGGDIQLTHVQ
-C terminal CRD (aa 227-354)
4-1BBL Protein Sequence:
MEYASDASLDPEAPWPPAPRARACRVLPWALVAGLLLLLLLAAACAVFLACPWAVSGARASPGSAAS
PRLREGPELSPDDPAGLLDLRQGMFAQLVAQNVLLIDGPLSWYSDPGLAGVSLTGGLSYKEDTKELV
VAKAGVYYVFFQLELRRVVAGEGSGSVSLALHLQPLRSAAGAAALALTVDLPPASSEARNSAFGFQG
RLLHLSAGQRLGVHLHTEARARHAWQLTQGATVLGLFRVTPEIPAGLPSPRSE
Tim-3 Protein Sequence:
MFSHLPFDCVLLLLLLLLTRSSEVEYRAEVGQNAYLPCFYTPAAPGNLVPVCWGKGACPVFECGN
VVLRTERDVNYWTSRYWLNGDFRKGDVSLTIENVTLADSGIYCCRIQIPGIMNDEKFNLKLVIKP
AKVTPAPTRQRDFTAAFPRMLTTRGHGPAETQTLGSLPDINLTQISTLANELRDSRLANDLRDSG
ATIRIGIYIGAGICAGLALALIFGALIFKWYSHSKEKIQNLSLISLANLPPSGLANAVAEGIRSE
ENIYTIEENVYEVEEPNEYYCYVSSRQQPSQPLGCRFAMP
CD44 Protein sequences:
Isoform 1:
MDKFWWHAAWGLCLVPLSLAQIDLNITCRFAGVFHVEKNGRYSISRTEAADLCKAFNSTLPTMAQ
MEKALSIGFETCRYGFIEGHVVIPRIHPNSICAANNTGVYILTSNTSQYDTYCFNASAPPEEDCT
SVTDLPNAFDGPITITIVNRDGTRYVQKGEYRTNPEDIYPSNPTDDDVSSGSSSERSSTSGGYIF
YTFSTVHPIPDEDSPWITDSTDRIPATTLMSTSATATETATKRQETWDWFSWLFLPSESKNHLHT
TTQMAGTSSNTISAGWEPNEENEDERDRHLSFSGSGIDDDEDFISSTISTTPRAFDHTKQNQDWT
QWNPSHSNPEVLLQTTTRMTDVDRNGTTAYEGNWNPEAHPPLIHHEHHEEEETPHSTSTIQATPS
STTEETATQKEQWFGNRWHEGYRQTPKEDSHSTTGTAAASAHTSHPMQGRTTPSPEDSSWTDFFN
PISHPMGRGHQAGRRMDMDSSHSITLQPTANPNTGLVEDLDRTGPLSMTTQQSNSQSFSTSHEGL
EEDKDHPTTSTLTSSNRNDVTGGRRDPNHSEGSTTLLEGYTSHYPHTKESRTFIPVTSAKTGSFG
VTAVTVGDSNSNVNRSLSGDQDTFHPSGGSHTTHGSESDGHSHGSQEGGANTTSGPIRTPQIPEW
LIILASLLALALILAVCIAVNSRRRCGQKKKLVINSGNGAVEDRKPSGLNEASKSQEMVHLVNKE
SSETPDQFMTADETRNLQNVDMKIGV
Isoform 2:
MDKFWWHAAWGLCLVPLSLAQIDLNITCRFAGVFHVEKNGRYSISRTEAADLCKAFNSTLPTMAQ
MEKALSIGFETCRYGFIEGHVVIPRIHPNSICAANNTGVYILTSNTSQYDTYCFNASAPPEEDCT
SVTDLPNAFDGPITITIVNRDGTRYVQKGEYRTNPEDIYPSNPTDDDVSSGSSSERSSTSGGYIF
YTFSTVHPIPDEDSPWITDSTDRIPATSTSSNTISAGWEPNEENEDERDRHLSFSGSGIDDDEDF
ISSTISTTPRAFDHTKQNQDWTQWNPSHSNPEVLLQTTTRMTDVDRNGTTAYEGNWNPEAHPPLI
HHEHHEEEETPHSTSTIQATPSSTTEETATQKEQWFGNRWHEGYRQTPKEDSHSTTGTAAASAHT
SHPMQGRTTPSPEDSSWTDFFNPISHPMGRGHQAGRRMDMDSSHSITLQPTANPNTGLVEDLDRT
GPLSMTTQQSNSQSFSTSHEGLEEDKDHPTTSTLTSSNRNDVTGGRRDPNHSEGSTTLLEGYTSH
YPHTKESRTFIPVTSAKTGSFGVTAVTVGDSNSNVNRSLSGDQDTFHPSGGSHTTHGSESDGHSH
GSQEGGANTTSGPIRTPQIPEWLIILASLLALALILAVCIAVNSRRRCGQKKKLVINSGNGAVED
RKPSGLNGEASKSQEMVHLVNKESSETPDQFMTADETRNLQNVDMKIGV
Isoform 3:
MDKFWWHAAWGLCLVPLSLAQIDLNITCRFAGVFHVEKNGRYSISRTEAADLCKAFNSTLPTMAQ
MEKALSIGFETCRYGFIEGHVVIPRIHPNSICAANNTGVYILTSNTSQYDTYCFNASAPPEEDCT
SVTDLPNAFDGPITITIVNRDGTRYVQKGEYRTNPEDIYPSNPTDDDVSSGSSSERSSTSGGYIF
YTFSTVHPIPDEDSPWITDSTDRIPATNMDSSHSITLQPTANPNTGLVEDLDRTGPLSMTTQQSN
SQSFSTSHEGLEEDKDHPTTSTLTSSNRNDVTGGRRDPNHSEGSTTLLEGYTSHYPHTKESRTFI
PVTSAKTGSFGVTAVTVGDSNSNVNRSLSGDQDTFHPSGGSHTTHGSESDGHSHGSQEGGANTTS
GPIRTPQIPEWLIILASLLALALILAVCIAVNSRRRCGQKKKLVINSGNGAVEDRKPSGLNGEAS
KSQEMVHLVNKESSETPDQFMTADETRNLQNVDMKIGV
Isoform 4:
MDKFWWHAAWGLCLVPLSLAQIDLNITCRFAGVFHVEKNGRYSISRTEAADLCKAFNSTLPTMAQ
MEKALSIGFETCRYGFIEGHVVIPRIHPNSICAANNTGVYILTSNTSQYDTYCFNASAPPEEDCT
SVTDLPNAFDGPITITIVNRDGTRYVQKGEYRTNPEDIYPSNPTDDDVSSGSSSERSSTSGGYIF
YTFSTVHPIPDEDSPWITDSTDRIPATRDQDTFHPSGGSHTTHGSESDGHSHGSQEGGANTTSGP
IRTPQIPEWLIILASLLALALILAVCIAVNSRRRCGQKKKLVINSGNGAVEDRKPSGLNGEASKS
QEMVHLVNKESSETPDQFMTADETRNLQNVDMKIGV
Isoform 5:
MDKFWWHAAWGLCLVPLSLAQIDLNITCRFAGVFHVEKNGRYSISRTEAADLCKAFNSTLPTMAQ
MEKALSIGFETCSLHCSQQSKKVWAEEKASDQQWQWSCGGQKAKWTQRRGQQVSGNGAFGEQGVV
RNSRPVYDS
Isoform 6:
MDKFWWHAAWGLCLVPLSLAQIDLNITCRFAGVFHVEKNGRYSISRTEAADLCKAFNSTLPTMAQ
MEKALSIGFETCRYGFIEGHVVIPRIHPNSICAANNTGVYILTSNTSQYDTYCFNASAPPEEDCT
SVTDLPNAFDGPITITIVNRDGTRYVQKGEYRTNPEDIYPSNPTDDDVSSGSSSERSSTSGGYIF
YTFSTVHPIPDEDSPWITDSTDRIPATNRNDVTGGRRDPNHSEGSTTLLEGYTSHYPHTKESRTF
IPVTSAKTGSFGVTAVTVGDSNSNVNRSLSGDQDTFHPSGGSHTTHGSESDGHSHGSQEGGANTT
SGPIRTPQIPEWLIILASLLALALILAVCIAVNSRRRCGQKKKLVINSGNGAVEDRKPSGLNGEA
SKSQEMVHLVNKESSETPDQFMTADETRNLQNVDMKIGV
Isoform 7
MDKFWWHAAWGLCLVPLSLAQIDLNITCRFAGVFHVEKNGRYSISRTEAADLCKAFNSTLPTMAQ
MEKALSIGFETCRYGFIEGHVVIPRIHPNSICAANNTGVYILTSNTSQYDTYCFNASAPPEEDCT
SVTDLPNAFDGPITITIVNRDGTRYVQKGEYRTNPEDIYPSNPTDDDVSSGSSSERSSTSGGYIF
YTFSTVHPIPDEDSPWITDSTDRIPATRHSHGSQEGGANTTSGPIRTPQIPEWLIILASLLALAL
ILAVCIAVNSRRRCGQKKKLVINSGNGAVEDRKPSGLNGEASKSQEMVHLVNKESSETPDQFMTA
DETRNLQNVDMKIGV
Isoform 8
MDKFWWHAAWGLCLVPLSLAQIDLNITCRFAGVFHVEKNGRYSISRTEAADLCKAFNSTLPTMAQ
MEKALSIGFETCRYGFIEGHVVIPRIHPNSICAANNTGVYILTSNTSQYDTYCFNASAPPEEDCT
SVTDLPNAFDGPITITIVNRDGTRYVQKGEYRTNPEDIYPSNPTDDDVSSGSSSERSSTSGGYIF
YTFSTVHPIPDEDSPWITDSTDRIPATRDQDTFHPSGGSHTTHGSESDGHSHGSQEGGANTTSGP
IRTPQIPEWLIILASLLALALILAVCIAVNSRRS

Non-limiting examples of peptides and fragments include, for 4-1BB, a cysteine rich domain (CRD), such as CRD 2 and/or CRD 3 of 4-1BB; or a 4-1BB peptide sequence: PCPPNSFSSAGGQRTCDICRQCKGVFRTRKECSSTSNAECDCTPGFHCLGAGCSMCEQDCKQGQELTKKGCKDC, or a subsequence thereof; or a 4-1BB peptide sequence: LQDPCSNCPAGTFCDNNRNQIC, PCPPNSFSSAGGQRTCDICRQCKGVFRTRKECSSTSNAEC, DCTPGFHCLGAGCSMCEQDCKQGQELTKKGCK, or DCCFGTFNDQKRGICRPWTNCSLDGKSVLVNGTKERDVVCG, or a subsequence thereof; or an extracellular domain sequence of human 4-1BB, from about amino acid residues 1-163 of human 4-1BB, or a subsequence thereof.

Non-limiting examples of peptides and fragments include, for 4-1BBL, from about residues 49-254 of 4-1BBL, 115 to 227 of 4-1BBL, from about 104-309 of 4-1BBL, 105-309 of 4-1BBL, or 106-309 of 4-1BBL, or a subsequence thereof.

Non-limiting examples of peptides and fragments include, for Galectin-9, a carbohydrate binding/recognition domain (CBD) of Galectin-9, or a subsequence of a carbohydrate binding/recognition domain (CBD) of Galectin-9. In particular examples for Galectin-9, a peptide or fragment of CBD1: PFSGTIQGGLQDGLQITVNGTVLSS SGTRFAVNFQTGFSGNDIAFHFNPRFEDGGYVVCNTRQNGSWGPEERKTHMPFQKGMPFDLCFLV QSSDFKVMVNGILFVQYFHRVPFHRVDTISVNGSVQLSYIS (residues from 16-146); or a peptide or fragment of CBD2: FITTILGGLYPSKSILLSGTVLPS AQRFHINLCSGNHIAFHLNPRFDENAVVRNTQIDNSWGSEERSLPRKMPFVRGQSFSVWILCEAH CLKVAVDGQHLFEYYHRLRNLPTINRLEVGGDIQLTHVQ (residues from 227-354); or a peptide or fragment of: PFSGTIQGGLQDGLQITVNGTVLSSSGTRFAVNFQTGFSGNDIAFHFNPRFEDGGYVVCNTRQNG SWGPEERKTHMPFQKGMPFDLCFLVQSSDFKVMVNGILFVQYFHRVPFHRVDTISVNGSVQLSYIS; or a peptide or fragment of: FITTILGGLYPSKSILLSGTVLPSAQRFHINLCSGNHIAFHLNPRFDENAVVRNTQIDNSWGSEE RSLPRKMPFVRGQSFSVWILCEAHCLKVAVDGQHLFEYYHRLRNLPTINRLEVGGDIQLTHVQ; or an N-terminal sequence portion of Galectin-9 from residues 50-58, 77-80, and/or 122-131 or a subsequence thereof; or a C-terminal sequence portion of Galectin-9 from residues 290-295, 310-314, and/or 326-336, or a subsequence thereof; or a contiguous Galectin-9 sequence comprising arginine at position 65.

Non-limiting examples of peptides and fragments include for Tim-3, all or a portion of residues 30-128 of human Tim-3.

Thus, in accordance with the invention, also provided are peptides and fragments that modulate binding of 4-1BB with the galectin. In particular embodiments, an invention peptide or fragment includes a peptide or fragment of 4-1BB, a peptide or fragment of a galectin such as Galectin-9, a peptide or fragment of a 4-1BB ligand, a peptide or fragment of a Tim-3, or a peptide or fragment of a CD44 polypeptide. More particular, such invention peptides and fragments include or consist of a fragment of galectin or Galectin-9 amino acid sequence that binds to a 4-1BB amino acid sequence; include or consist of a fragment of galectin or Galectin-9 amino acid sequence that binds to a Tim-3 amino acid sequence; include or consist of a fragment of galectin or Galectin-9 amino acid sequence that binds to a CD44 amino acid sequence; include or consist of a fragment of a 4-1BB amino acid sequence that binds to a galectin or Galectin-9 amino acid sequence; include or consist of a fragment of a 4-1BB amino acid sequence that binds to a 4-1BBL amino acid sequence; include or consist of a fragment of a 4-1BBL amino acid sequence that binds to a 4-1BB amino acid sequence; include or consist of a fragment of a Tim-3 amino acid sequence that binds to a Galectin-9 amino acid sequence and include or consist of a fragment of a CD44 amino acid sequence that binds to a Galectin-9 amino acid sequence.

Proteins and antibodies, as well as subsequences and fragments thereof, can be produced by genetic methodology. Such techniques include expression of all or a part of the gene encoding the protein or antibody into a host cell such as Cos cells or E. coli. The recombinant host cells synthesize full length or a subsequence, for example, an scFv (see, e.g., Whitlow et al., In: Methods: A Companion to Methods in Enzymology 2:97 (1991), Bird et al., Science 242:423 (1988); and U.S. Pat. No. 4,946,778). Single-chain Fvs and antibodies can be produced as described in U.S. Pat. Nos. 4,946,778 and 5,258,498; Huston et al., Methods Enzymol. 203:46 (1991); Shu et al., Proc. Natl. Acad. Sci. USA 90:7995 (1993); and Skerra et al., Science 240:1038 (1988).

The terms “fusion” or “chimeric” and grammatical variations thereof, when used in reference to a “polypeptide,” or “protein” or “peptide” means that a portion or part of the molecule contains a different entity distinct (heterologous) from the molecule (e.g., 4-1BB, the galectin, 4-1BB ligand, Tim-3 or CD44 fragment or antibody) as they do not typically exist together in nature. That is, for example, one portion of the fusion or chimera, such as 4-1BB, the galectin, 4-1BB ligand, Tim-3 or CD44, includes or consists of a portion that does not exist together in nature, and is structurally distinct. A particular example is a molecule, such as an amino acid sequence of another protein (e.g., immunoglobulin such as an Fc domain, or antibody) attached to 4-1BB, the galectin, 4-1BB ligand, Tim-3 or CD44 to produce a chimera, or a chimeric polypeptide, to impart a distinct function (e.g., increased solubility, in vivo half life, etc.). Another particular example is an amino acid sequence of another protein to produce a multifunctional protein (e.g., multifunctional 4-1BB, the galectin, 4-1BB ligand, Tim-3 or CD44 or multispecific antibody that binds to 4-1BB, the galectin, 4-1BB ligand, Tim-3 or CD44).

In still further embodiments, the agent that modulates binding of 4-1BB with the galectin is an inhibitory nucleic acid that reduces expression or activity of 4-1BB, the galectin, 4-1BB ligand, Tim-3 or CD44. In different embodiments, the inhibitory nucleic acid comprises an antisense, triplex forming RNA, small interfering RNA (siRNA) or micro RNA (miRNA).

As used herein, the terms “nucleic acid,” “polynucleotide” and “polynucleoside” are used interchangeably to refer to all forms of nucleic acid, oligonucleotides, primers, and probes, including deoxyribonucleic acid (DNA) and ribonucleic acid (RNA). Nucleic acid includes genomic DNA, cDNA and antisense DNA, and spliced or unspliced mRNA, rRNA tRNA and antisense RNA (e.g., RNAi). Nucleic acids include naturally occurring, synthetic, and intentionally altered or modified polynucleotides as well as analogues and derivatives. Alterations can result in increased stability due to resistance to nuclease digestion, for example. Nucleic acids can be double, single or triplex, linear or circular, and can be of any length.

Nucleic acids include sequences that are degenerate as a result of the genetic code. There are 20 natural amino acids, most of which are specified by more than one codon. Degenerate sequences may not selectively hybridize to other nucleic acids; however, they are nonetheless included.

Nucleic acid sequences include sequences having 10-15, 15-20, 20-30, 30-40, 50-60, 60-75, 75-100, 100-125, 125-150, 150-200, or more contiguous nucleotides. In additional aspects, the nucleic acid sequence includes a sequence having 60 or more, 70 or more, 80 or more, 100 or more, 120 or more, 140 or more, 160 or more contiguous nucleotides, up to the full length coding sequence.

Nucleic acid sequences include complementary sequences (e.g., inhibitory nucleic acid such as an antisense) to all or a part of 4-1BB, 4-1BBL, a galectin (e.g., Galectin-9), Tim-3, or CD44 encoding gene or transcript (e.g. preRNA or mRNA). Inhibitory, antisense and RNAi nucleic acids can modulate expression of 4-1BB, 4-1BBL, a galectin (e.g., Galectin-9), Tim-3, or CD44.

Inhibitory nucleic acid includes single, double or triple stranded polynucleotides/polynucleosides and peptide nucleic acids (PNAs) that bind RNA transcript or DNA (e.g., genomic DNA). For example, a single stranded nucleic acid can target 4-1BB, 4-1BBL, a galectin (e.g., Galectin-9), Tim-3, or CD44 transcript (e.g., mRNA). Oligonucleotides derived from the transcription initiation site of 4-1BB, 4-1BBL, a galectin (e.g., Galectin-9), Tim-3, or CD44 gene, e.g., between positions −10 and +10 from the start site, are another particular example. Triplex forming antisense can bind to double strand DNA thereby inhibiting transcription of the gene.

“RNAi” is the use of double stranded RNA sequences for inhibiting gene expression (see, e.g., Kennerdell et al., Cell 95:1017 (1998); and Fire et al., Nature, 391:806 (1998)). Double stranded RNA sequences from a 4-1BB, 4-1BBL, a galectin (e.g., Galectin-9), Tim-3, or CD44 coding region may therefore be used to inhibit or prevent 4-1BB, 4-1BBL, a galectin (e.g., Galectin-9), Tim-3, or CD44 expression in accordance with the methods and uses of the invention. Antisense and RNAi can be produced based upon nucleic acids encoding 4-1BB, 4-1BBL, a galectin (e.g., Galectin-9), Tim-3, or CD44 sequences.

Inhibitory nucleic acid (e.g., antisense) may be encoded by a nucleic acid, and such a nucleic acid may be operatively linked to an expression control element for sustained or increased expression of the encoded antisense in cells or in vivo. Such inhibitory nucleic acid can be included with an expression control element controlling expression of a nucleic acid can be modified or altered.

Inhibitory nucleic acid can be based upon genomic 4-1BB, 4-1BBL, a galectin (e.g., Galectin-9), Tim-3, or CD44 sequences, or cDNA sequences that encode 4-1BB, 4-1BBL, a galectin (e.g., Galectin-9), Tim-3, or CD44 sequences. The following are representative 4-1BB, 4-1BBL, Galectin-9, Tim-3, and CD44 sequences:

4-1BB Sequence:
CAAGGAGGGATCCCACAGATGTCACAGGGCTGTCACAGAGCTGTGGTGGGAATTTCCCATGAGAC
CCCGCCCCTGGCTGAGTCACCGCACTCCTGTGTTTGACCTGAAGTCCTCTCGAGCTGCAGAAGCC
TGAAGACCAAGGAGTGGAAAGTTCTCCGGCAGCCCTGAGATCTCAAGAGTGACATTTGTGAGACC
AGCTAATTTGATTAAAATTCTCTTGGAATCAGCTTTGCTAGTATCATACCTGTGCCAGATTTCAT
CATGGGAAACAGCTGTTACAACATAGTAGCCACTCTGTTGCTGGTCCTCAACTTTGAGAGGACAA
GATCATTGCAGGATCCTTGTAGTAACTGCCCAGCTGGTACATTCTGTGATAATAACAGGAATCAG
ATTTGCAGTCCCTGTCCTCCAAATAGTTTCTCCAGCGCAGGTGGACAAAGGACCTGTGACATATG
CAGGCAGTGTAAAGGTGTTTTCAGGACCAGGAAGGAGTGTTCCTCCACCAGCAATGCAGAGTGTG
ACTGCACTCCAGGGTTTCACTGCCTGGGGGCAGGATGCAGCATGTGTGAACAGGATTGTAAACAA
GGTCAAGAACTGACAAAAAAAGGTTGTAAAGACTGTTGCTTTGGGACATTTAACGATCAGAAACG
TGGCATCTGTCGACCCTGGACAAACTGTTCTTTGGATGGAAAGTCTGTGCTTGTGAATGGGACGA
AGGAGAGGGACGTGGTCTGTGGACCATCTCCAGCCGACCTCTCTCCGGGAGCATCCTCTGTGACC
CCGCCTGCCCCTGCGAGAGAGCCAGGACACTCTCCGCAGATCATCTCCTTCTTTCTTGCGCTGAC
GTCGACTGCGTTGCTCTTCCTGCTGTTCTTCCTCACGCTCCGTTTCTCTGTTGTTAAACGGGGCA
GAAAGAAACTCCTGTATATATTCAAACAACCATTTATGAGACCAGTACAAACTACTCAAGAGGAA
GATGGCTGTAGCTGCCGATTTCCAGAAGAAGAAGAAGGAGGATGTGAACTGTGAAATGGAAGTCA
ATAGGGCTGTTGGGACTTTCTTGAAAAGAAGCAAGGAAATATGAGTCATCCGCTATCACAGCTTT
CAAAAGCAAGAACACCATCCTACATAATACCCAGGATTCCCCCAACACACGTTCTTTTCTAAATG
CCAATGAGTTGGCCTTTAAAAATGCACCACTTTTTTTTTTTTTTTGACAGGGTCTCACTCTGTCA
CCCAGGCTGGAGTGCAGTGGCACCACCATGGCTCTCTGCAGCCTTGACCTCTGGGAGCTCAAGTG
ATCCTCCTGCCTCAGTCTCCTGAGTAGCTGGAACTACAAGGAAGGGCCACCACACCTGACTAACT
TTTTTGTTTTTTGTTTGGTAAAGATGGCATTTCACCATGTTGTACAGGCTGGTCTCAAACTCCTA
GGTTCACTTTGGCCTCCCAAAGTGCTGGGATTACAGACATGAACTGCCAGGCCCGGCCAAAATAA
TGCACCACTTTTAACAGAACAGACAGATGAGGACAGAGCTGGTGATAAAAAAAAAAAAAAAAAAG
CATTTTCTAGATACCACTTAACAGGTTTGAGCTAGTTTTTTTGAAATCCAAAGAAAATTATAGTT
TAAATTCAATTACATAGTCCAGTGGTCCAACTATAATTATAATCAAAATCAATGCAGGTTTGTTT
TTTGGTGCTAATATGACATATGACAATAAGCCACGAGGTGCAGTAAGTACCCGACTAAAGTTTCC
GTGGGTTCTGTCATGTAACACGACATGCTCCACCGTCAGGGGGGAGTATGAGCAGAGTGCCTGAG
TTTAGGGTCAAGGACAAAAAACCTCAGGCCTGGAGGAAGTTTTGGAAAGAGTTCAAGTGTCTGTA
TATCCTATGGTCTTCTCCATCCTCACACCTTCTGCCTTTGTCCTGCTCCCTTTTAAGCCAGGTTA
CATTCTAAAAATTCTTAACTTTTAACATAATATTTTATACCAAAGCCAATAAATGAACTGCATAT
GATAGGTATGAAGTACAGTGAGAAAATTAACACCTGTGAGCTCATTGTCCTACCACAGCACTAGA
GTGGGGGCCGCCAAACTCCCATGGCCAAACCTGGTGCACCATTTGCCTTTGTTTGTCTGTTGGTT
TGCTTGAGACAGTCTTGCTCTGTTGCCCAGGCTGGAATGGAGTGGCTATTCACAGGCACAATCAT
AGCACACTTTAGCCTTAAACTCCTGGGCTCAAGTGATCCACCCGCCTCAGTCTCCCAAGTAGCTG
GGATTACAGGTGCAAACCTGGCATGCCTGCCATTGTTTGGCTTATGATCTAAGGATAGCTTTTTA
AATTTTATTCATTTTATTTTTTTTTGAGACAGTGTCTCACTCTGTCTCCCAGGCTGGAGTACAGT
GGTACAATCTTGGATCACCGCCTCCCAGTTTCAAGTGATCTCCCTGCCTCAGCCTCCTAAGTAGC
TGGGACTACAGGTATGTGCCACCACGCCTGGCTAATTTTTATATTTTTAGTAGAGACGGGGTTTC
ACCATGTTGTCCAGGCTGGTCTCAAACTCCTGACCTCAGGTGATCTGCCCACCTCTGCCTCCCAA
AGTGCTGGGATTACAGGCATGAGCCACCATGCCTGGCCATTTCTTACACTTTTGTATGACATGCC
TATTGCAAGCTTGCGTGCCTCTGTCCCATGTTATTTTACTCTGGGATTTAGGTGGAGGGAGCAGC
TTCTATTTGGAACATTGGCCATCGCATGGCAAATGGGTATCTGTCACTTCTGCTCCTATTTAGTT
GGTTCTACTATAACCTTTAGAGCAAATCCTGCAGCCAAGCCAGGCATCAATAGGGCAGAAAAGTA
TATTCTGTAAATAGGGGTGAGGAGAAGATATTTCTGAACAATAGTCTACTGCAGTACCAAATTGC
TTTTCAAAGTGGCTGTTCTAATGTACTCCCGTCAGTCATATAAGTGTCATGTAAGTATCCCATTG
ATCCACATCCTTGCTACCCTCTGGTACTATCAGGTGCCCTTAATTTTGCCAAGCCAGTGGGTATA
GAATGAGATCTCACTGTGGTCTTAGTTTGCATTTGCTTGGTTACTGATGAGCACCTTGTCAAATA
TTTATATACCATTTGTGTTTATTTTTTTAAATAAAATGCTTGCTCATGCTTTTTTGCCCATTTGC
AAAAAAACTTGGGGCCGGGTGCAGTGGCTCATGCCTGTAGTCCCAGCTCTTTGGGAGGCCAAGGT
GGGCAGATCGCTTGAGCCCAGGAGTTCGAGACCAGCCTTGGCAACATGGCGAAACCCTGTCTTTA
CAAAAAATACAAAAATTAGCCGGGTGTGGTGGTGTGCACCTGAAGTCCCAGCTACTCAGTAGGTT
CGCTTTGAGCCTGGGAGGCAGAGGTTGCAGTGAGCTGGGACCGCATCACTACACTTCAGCCTGGG
CAACAGAGAAAAACCTTTTCTCAGAAACAAACAAACCCAAATGTGGTTGTTTGTCCTGATTCCTA
AAAGGTCTTTATGTATTCTAGATAATAATCTTTGGTCAGTTATATGTGTTAAAAAATATCTTCTT
TGTGGCCAGGCACGGTAGCTCACACCTGTAATCCCAGCACTTTGCGGGGCTGAGGTGGGTGGATC
ATCTGAGGTCAAGAGTTCAAGATCAGCCTGGCCAACACAGTGAAACCCCATCTCTACTAAACATG
TACAAAACTTAGCTGGGTATGGTGGCGGGTGCCTGTAACCCCAGCTGCTCCAGAGGCTGTGGCAG
AAGAATCGCTTGAACCCAGGAGGCAGAGGTTGCAGCGAGCCAAGATTGTGCCATTGCACTCCAGA
CTGGGTGACAAGAGTGAAATTCTGCCTATCTATCTATCTATCTATCTATATCTATATATATATAT
ATATATATCCTTTGTAATTTATTTTTCCCTTTTTAAAATTTTTTATAAAATTCTTTTTTATTTTT
ATTTTTAGCAGAGGTGAGGTTTCTGAGGTTTCATTATGTTGCCCAGGCTGGTCTTGAACTCCTGA
GCTCAAGTGATCCTCCCACCTCAGCCTTCCAAAGTGCTGGAATTGCAGACATGAGCCACCGCGCC
CCTCCTGTTTTTCTCTAATTAATGGTGTCTTTCTTTGTCTTTCTGGTAATAAGCAAAAAGTTCTT
CATTTGATTTGGTTAAATTTATAACTGTTTTCTCATATGGTTAACATTTTTTCTTGCCTGGCTAA
AGAAATCCTTTTCTGCCCAATACTATAAAGAGGTTTGCCCACATTTTATTCCAAAAGTTTTAAGT
TTTGTCTTTCATCTTGAAGTCTAATGTATCAGGAACTGGCTTTTGTGCCTGTTGGGAGGTAGTGA
TCCAATTCCATGTCTTGCATGTAGGTAACCACTGGTCCCTGCGCCATGTATTCAATACGTCGTCT
TTCTCCTGCGGGTCTGCAATCTCACCTACCATCCATCAAGTTTCCATAGGGCCATGGGTCTGCTT
CTGGGCTCCCTGTTCTGTTCCATTGTCAATTTGTCTATCCTGTGCCAGTATCACACTGTGTTTAT
TACAATAGCTTTGTAACAGCTCTCGATATCCGGTAGGACATCTCCCTCCACCTTCTTTTTCTACT
TCAGAAGTGTCTTAGCTAGGTCAGGCACGGTGGCTCACGCCTGTAATCCCAGCACTTTGGGAGGC
CGACGCGGATGGATCACCTGAGGTCAGGAGTTTTGAGACAGCCTGGCCAACATGGTGAAACCCCA
TCTCTACTAAAAAATACAAAAATTAGTCAGGCATGGTGGCATGTGCCTGTAATCCCAGCTATTTG
GGAGGCTGAGGCCGGAGAATTGCTTGAACCCGGGGGGCGGAGGTTGCAGTGAGCCGAGATCGTAC
CATTGCACTCCAGCCTGGGTGACAGAGCGAAACTCTGTCTCAGGAAAAAAAAGAAAAGAGATGTC
TTGGTTATTCTTGGTTCTTTATTATTCAATATAAATTTTAGAAGCTGAATTTGAAAAGATTTGGA
TTGGAATTTCATTAAATCTACAGGTCAATTTAGGGAGAGTTGATAATTTTACAGAATTGAGTCAT
CTGGTGTTCCAATAAGAATAAGAGAACAATTATTGGCTGTACAATTCTTGCCAAATAGTAGGCAA
AGCAAAGCTTAGGAAGTATACTGGTGCCATTTCAGGAACAAAGCTAGGTGCGAATATTTTTGTCT
TTCTGAATCATGATGCTGTAAGTTCTAAAGTGATTTCTCCTCTTGGCTTTGGACACATGGTGTTT
AATTACCTACTGCTGACTATCCACAAACAGAAAGAGACTGGTCATGCCCCACAGGGTTGGGGTAT
CCAAGATAATGGAGCGAGGCTCTCATGTGTCCTAGGTTACACACCGAAAATCCACAGTTTATTCT
GTGAAGAAAGGAGGCTATGTTTATGATACAGACTGTGATATTTTTATCATAGCCTATTCTGGTAT
CATGTGCAAAAGCTATAAATGAAAAACACAGGAACTTGGCATGTGAGTCATTGCTCCCCCTAAAT
GACAATTAATAAGGAAGGAACATTGAGACAGAATAAAATGATCCCCTTCTGGGTTTAATTTAGAA
AGTTCCATAATTAGGTTTAATAGAAATAAATGTAAATTTCTATGATTAAAAATAAATTAGCACAT
TTAGGGATACACAAATTATAAATCATTTTCTAAATGCTAAAAACAAGCTCAGGTTTTTTTCAGAA
GAAAGTTTTAATTTTTTTTCTTTAGTGGAAGATATCACTCTGACGGAAAGTTTTGATGTGAGGGG
CGGATGACTATAAAGTGGGCATCTTCCCCCACAGGAAGATGTTTCCATCTGTGGGTGAGAGGTGC
CCACCGCAGCTAGGGCAGGTTACATGTGCCCTGTGTGTGGTAGGACTTGGAGAGTGATCTTTATC
AACGTTTTTATTTAAAAGACTATCTAATAAAACACAAAACTATGATGTTCACAGGAAAAAAAGAA
TAAGAAAAAAAGAAAAAAAAA
4-1BBL Sequence:
AAAAAGCGGCGCGCTGTGTCTTCCCGCAGTCTCTCGTCATGGAATACGCCTCTGACGCTTCACTG
GACCCCGAAGCCCCGTGGCCTCCCGCGCCCCGCGCTCGCGCCTGCCGCGTACTGCCTTGGGCCCT
GGTCGCGGGGCTGCTGCTGCTGCTGCTGCTCGCTGCCGCCTGCGCCGTCTTCCTCGCCTGCCCCT
GGGCCGTGTCCGGGGCTCGCGCCTCGCCCGGCTCCGCGGCCAGCCCGAGACTCCGCGAGGGTCCC
GAGCTTTCGCCCGACGATCCCGCCGGCCTCTTGGACCTGCGGCAGGGCATGTTTGCGCAGCTGGT
GGCCCAAAATGTTCTGCTGATCGATGGGCCCCTGAGCTGGTACAGTGACCCAGGCCTGGCAGGCG
TGTCCCTGACGGGGGGCCTGAGCTACAAAGAGGACACGAAGGAGCTGGTGGTGGCCAAGGCTGGA
GTCTACTATGTCTTCTTTCAACTAGAGCTGCGGCGCGTGGTGGCCGGCGAGGGCTCAGGCTCCGT
TTCACTTGCGCTGCACCTGCAGCCACTGCGCTCTGCTGCTGGGGCCGCCGCCCTGGCTTTGACCG
TGGACCTGCCACCCGCCTCCTCCGAGGCTCGGAACTCGGCCTTCGGTTTCCAGGGCCGCTTGCTG
CACCTGAGTGCCGGCCAGCGCCTGGGCGTCCATCTTCACACTGAGGCCAGGGCACGCCATGCCTG
GCAGCTTACCCAGGGCGCCACAGTCTTGGGACTCTTCCGGGTGACCCCCGAAATCCCAGCCGGAC
TCCCTTCACCGAGGTCGGAATAACGTCCAGCCTGGGTGCAGCCCACCTGGACAGAGTCCGAATCC
TACTCCATCCTTCATGGAGACCCCTGGTGCTGGGTCCCTGCTGCTTTCTCTACCTCAAGGGGCTT
GGCAGGGGTCCCTGCTGCTGACCTCCCCTTGAGGACCCTCCTCACCCACTCCTTCCCCAAGTTGG
ACCTTGATATTTATTCTGAGCCTGAGCTCAGATAATATATTATATATATTATATATATATATATA
TTTCTATTTAAAGAGGATCCTGAGTTTGTGAATGGACTTTTTTAGAGGAGTTGTTTTGGGGGGGG
GGGGGTCTTCGACATTGCCGAGGCTGGTCTTGAACTCCTGGACTTAGACGATCCTCCTGCCTCAG
CCTCCCAAGCAACTGGGATTCATCCTTTCTATTAATTCATTGTACTTATTTGCTTATTTGTGTGT
ATTGAGCATCTGTAATGTGCCAGCATTGTGCCCAGGCTAGGGGGCTATAGAAACATCTAGAAATA
GACTGAAAGAAAATCTGAGTTATGGTAATACGTGAGGAATTTAAAGACTCATCCCCAGCCTCCAC
CTCCTGTGTGATACTTGGGGGCTAGCTTTTTTCTTTCTTTCTTTTTTTTGAGATGGTCTTGTTCT
GTCAACCAGGCTAGAATGCAGCGGTGCAATCATGAGTCAATGCAGCCTCCAGCCTCGACCTCCCG
AGGCTCAGGTGATCCTCCCATCTCAGCCTCTCGAGTAGCTGGGACCACAGTTGTGTGCCACCACA
CTTGGCTAACTTTTTAATTTTTTTGCGGAGACGGTATTGCTATGTTGCCAAGGTTGTTTACATGC
CAGTACAATTTATAATAAACACTCATTTTTCCTCCCTCTGAAAAAAAAAAAAAAA
Galectin-9 Sequence:
TCCCCATTGAATAACAGCCAAGTTGCTTTGGTTTCTATTTCTTTGTTAAGTCGTTCCCTCTACAA
AGGACTTCCTAGTGGGTGTGAAAGGCAGCGGTGGCCACAGAGGCGGCGGAGAGATGGCCTTCAGC
GGTTCCCAGGCTCCCTACCTGAGTCCAGCTGTCCCCTTTTCTGGGACTATTCAAGGAGGTCTCCA
GGACGGACTTCAGATCACTGTCAATGGGACCGTTCTCAGCTCCAGTGGAACCAGGTTTGCTGTGA
ACTTTCAGACTGGCTTCAGTGGAAATGACATTGCCTTCCACTTCAACCCTCGGTTTGAAGATGGA
GGGTACGTGGTGTGCAACACGAGGCAGAACGGAAGCTGGGGGCCCGAGGAGAGGAAGACACACAT
GCCTTTCCAGAAGGGGATGCCCTTTGACCTCTGCTTCCTGGTGCAGAGCTCAGATTTCAAGGTGA
TGGTGAACGGGATCCTCTTCGTGCAGTACTTCCACCGCGTGCCCTTCCACCGTGTGGACACCATC
TCCGTCAATGGCTCTGTGCAGCTGTCCTACATCAGCTTCCAGAACCCCCGCACAGTCCCTGTTCA
GCCTGCCTTCTCCACGGTGCCGTTCTCCCAGCCTGTCTGTTTCCCACCCAGGCCCAGGGGGCGCA
GACAAAAACCTCCCGGCGTGTGGCCTGCCAACCCGGCTCCCATTACCCAGACAGTCATCCACACA
GTGCAGAGCGCCCCTGGACAGATGTTCTCTACTCCCGCCATCCCACCTATGATGTACCCCCACCC
CGCCTATCCGATGCCTTTCATCACCACCATTCTGGGAGGGCTGTACCCATCCAAGTCCATCCTCC
TGTCAGGCACTGTCCTGCCCAGTGCTCAGAGGTTCCACATCAACCTGTGCTCTGGGAACCACATC
GCCTTCCACCTGAACCCCCGTTTTGATGAGAATGCTGTGGTCCGCAACACCCAGATCGACAACTC
CTGGGGGTCTGAGGAGCGAAGTCTGCCCCGAAAAATGCCCTTCGTCCGTGGCCAGAGCTTCTCAG
TGTGGATCTTGTGTGAAGCTCACTGCCTCAAGGTGGCCGTGGATGGTCAGCACCTGTTTGAATAC
TACCATCGCCTGAGGAACCTGCCCACCATCAACAGACTGGAAGTGGGGGGCGACATCCAGCTGAC
CCATGTGCAGACATAGGCGGCTTCCTGGCCCTGGGGCCGGGGGCTGGGGTGTGGGGCAGTCTGGG
TCCTCTCATCATCCCCACTTCCCAGGCCCAGCCTTTCCAACCCTGCCTGGGATCTGGGCTTTAAT
GCAGAGGCCATGTCCTTGTCTGGTCCTGCTTCTGGCTACAGCCACCCTGGAACGGAGAAGGCAGC
TGACGGGGATTGCCTTCCTCAGCCGCAGCAGCACCTGGGGCTCCAGCTGCTGGAATCCTACCATC
CCAGGAGGCAGGCACAGCCAGGGAGAGGGGAGGAGTGGGCAGTGAAGATGAAGCCCCATGCTCAG
TCCCCTCCCATCCCCCACGCAGCTCCACCCCAGTCCCAAGCCACCAGCTGTCTGCTCCTGGTGGG
AGGTGGCCTCCTCAGCCCCTCCTCTCTGACCTTTAACCTCACTCTCACCTTGCACCGTGCACCAA
CCCTTCACCCCTCCTGGAAAGCAGGCCTGATGGCTTCCCACTGGCCTCCACCACCTGACCAGAGT
GTTCTCTTCAGAGGACTGGCTCCTTTCCCAGTGTCCTTAAAATAAAGAAATGAAAATGCTTGTTG
GCACATTCAAAAAAAAAAAAAAAAAAAAAAAAAAAAAAAAAAAAAAAAAAAAAAAAAAAAAAAAA
AAAAAAAAAAAAAAA
Tim-3 mRNA Sequence:
AGAACACTTACAGGATGTGTGTAGTGTGGCATGACAGAGAACTTTGGTTTCCTTTAATGTGACTG
TAGACCTGGCAGTGTTACTATAAGAATCACTGGCAATCAGACACCCGGGTGTGCTGAGCTAGCAC
TCAGTGGGGGCGGCTACTGCTCATGTGATTGTGGAGTAGACAGTTGGAAGAAGTACCCAGTCCAT
TTGGAGAGTTAAAACTGTGCCTAACAGAGGTGTCCTCTGACTTTTCTTCTGCAAGCTCCATGTTT
TCACATCTTCCCTTTGACTGTGTCCTGCTGCTGCTGCTGCTACTACTTACAAGGTCCTCAGAAGT
GGAATACAGAGCGGAGGTCGGTCAGAATGCCTATCTGCCCTGCTTCTACACCCCAGCCGCCCCAG
GGAACCTCGTGCCCGTCTGCTGGGGCAAAGGAGCCTGTCCTGTGTTTGAATGTGGCAACGTGGTG
CTCAGGACTGATGAAAGGGATGTGAATTATTGGACATCCAGATACTGGCTAAATGGGGATTTCCG
CAAAGGAGATGTGTCCCTGACCATAGAGAATGTGACTCTAGCAGACAGTGGGATCTACTGCTGCC
GGATCCAAATCCCAGGCATAATGAATGATGAAAAATTTAACCTGAAGTTGGTCATCAAACCAGCC
AAGGTCACCCCTGCACCGACTCGGCAGAGAGACTTCACTGCAGCCTTTCCAAGGATGCTTACCAC
CAGGGGACATGGCCCAGCAGAGACACAGACACTGGGGAGCCTCCCTGATATAAATCTAACACAAA
TATCCACATTGGCCAATGAGTTACGGGACTCTAGATTGGCCAATGACTTACGGGACTCTGGAGCA
ACCATCAGAATAGGCATCTACATCGGAGCAGGGATCTGTGCTGGGCTGGCTCTGGCTCTTATCTT
CGGCGCTTTAATTTTCAAATGGTATTCTCATAGCAAAGAGAAGATACAGAATTTAAGCCTCATCT
CTTTGGCCAACCTCCCTCCCTCAGGATTGGCAAATGCAGTAGCAGAGGGAATTCGCTCAGAAGAA
AACATCTATACCATTGAAGAGAACGTATATGAAGTGGAGGAGCCCAATGAGTATTATTGCTATGT
CAGCAGCAGGCAGCAACCCTCACAACCTTTGGGTTGTCGCTTTGCAATGCCATAGATCCAACCAC
CTTATTTTTGAGCTTGGTGTTTTGTCTTTTTCAGAAACTATGAGCTGTGTCACCTGACTGGTTTT
GGAGGTTCTGTCCACTGCTATGGAGCAGAGTTTTCCCATTTTCAGAAGATAATGACTCACATGGG
AATTGAACTGGGACCTGCACTGAACTTAAACAGGCATGTCATTGCCTCTGTATTTAAGCCAACAG
AGTTACCCAACCCAGAGACTGTTAATCATGGATGTTAGAGCTCAAACGGGCTTTTATATACACTA
GGAATTCTTGACGTGGGGTCTCTGGAGCTCCAGGAAATTCGGGCACATCATATGTCCATGAAACT
TCAGATAAACTAGGGAAAACTGGGTGCTGAGGTGAAAGCATAACTTTTTTGGCACAGAAAGTCTA
AAGGGGCCACTGATTTTCAAAGAGATCTGTGATCCCTTTTTGTTTTTTGTTTTTGAGATGGAGTC
TTGCTCTGTTGCCCAGGCTGGAGTGCAATGGCACAATCTCGGCTCACTGCAAGCTCCGCCTCCTG
GGTTCAAGCGATTCTCCTGCCTCAGCCTCCTGAGTGGCTGGGATTACAGGCATGCACCACCATGC
CCAGCTAATTTGTTGTATTTTTAGTAGAGACAGGGTTTCACCATGTTGGCCAGTGTGGTCTCAAA
CTCCTGACCTCATGATTTGCCTGCCTCGGCCTCCCAAAGCACTGGGATTACAGGCGTGAGCCACC
ACATCCAGCCAGTGATCCTTAAAAGATTAAGAGATGACTGGACCAGGTCTACCTTGATCTTGAAG
ATTCCCTTGGAATGTTGAGATTTAGGCTTATTTGAGCACTGCCTGCCCAACTGTCAGTGCCAGTG
CATAGCCCTTCTTTTGTCTCCCTTATGAAGACTGCCCTGCAGGGCTGAGATGTGGCAGGAGCTCC
CAGGGAAAAACGAAGTGCATTTGATTGGTGTGTATTGGCCAAGTTTTGCTTGTTGTGTGCTTGAA
AGAAAATATCTCTGACCAACTTCTGTATTCGTGGACCAAACTGAAGCTATATTTTTCACAGAAGA
AGAAGCAGTGACGGGGACACAAATTCTGTTGCCTGGTGGAAAGAAGGCAAAGGCCTTCAGCAATC
TATATTACCAGCGCTGGATCCTTTGACAGAGAGTGGTCCCTAAACTTAAATTTCAAGACGGTATA
GGCTTGATCTGTCTTGCTTATTGTTGCCCCCTGCGCCTAGCACAATTCTGACACACAATTGGAAC
TTACTAAAAATTTTTTTTTACTGTTAAAAAAAAAAAAAAAAAA
CD44 Sequence:
GAGAAGAAAGCCAGTGCGTCTCTGGGCGCAGGGGCCAGTGGGGCTCGGAGGCACAGGCACCCCGC
GACACTCCAGGTTCCCCGACCCACGTCCCTGGCAGCCCCGATTATTTACAGCCTCAGCAGAGCAC
GGGGCGGGGGCAGAGGGGCCCGCCCGGGAGGGCTGCTACTTCTTAAAACCTCTGCGGGCTGCTTA
GTCACAGCCCCCCTTGCTTGGGTGTGTCCTTCGCTCGCTCCCTCCCTCCGTCTTAGGTCACTGTT
TTCAACCTCGAATAAAAACTGCAGCCAACTTCCGAGGCAGCCTCATTGCCCAGCGGACCCCAGCC
TCTGCCAGGTTCGGTCCGCCATCCTCGTCCCGTCCTCCGCCGGCCCCTGCCCCGCGCCCAGGGAT
CCTCCAGCTCCTTTCGCCCGCGCCCTCCGTTCGCTCCGGACACCATGGACAAGTTTTGGTGGCAC
GCAGCCTGGGGACTCTGCCTCGTGCCGCTGAGCCTGGCGCAGATCGATTTGAATATAACCTGCCG
CTTTGCAGGTGTATTCCACGTGGAGAAAAATGGTCGCTACAGCATCTCTCGGACGGAGGCCGCTG
ACCTCTGCAAGGCTTTCAATAGCACCTTGCCCACAATGGCCCAGATGGAGAAAGCTCTGAGCATC
GGATTTGAGACCTGCAGGTATGGGTTCATAGAAGGGCACGTGGTGATTCCCCGGATCCACCCCAA
CTCCATCTGTGCAGCAAACAACACAGGGGTGTACATCCTCACATCCAACACCTCCCAGTATGACA
CATATTGCTTCAATGCTTCAGCTCCACCTGAAGAAGATTGTACATCAGTCACAGACCTGCCCAAT
GCCTTTGATGGACCAATTACCATAACTATTGTTAACCGTGATGGCACCCGCTATGTCCAGAAAGG
AGAATACAGAACGAATCCTGAAGACATCTACCCCAGCAACCCTACTGATGATGACGTGAGCAGCG
GCTCCTCCAGTGAAAGGAGCAGCACTTCAGGAGGTTACATCTTTTACACCTTTTCTACTGTACAC
CCCATCCCAGACGAAGACAGTCCCTGGATCACCGACAGCACAGACAGAATCCCTGCTACCACTTT
GATGAGCACTAGTGCTACAGCAACTGAGACAGCAACCAAGAGGCAAGAAACCTGGGATTGGTTTT
CATGGTTGTTTCTACCATCAGAGTCAAAGAATCATCTTCACACAACAACACAAATGGCTGGTACG
TCTTCAAATACCATCTCAGCAGGCTGGGAGCCAAATGAAGAAAATGAAGATGAAAGAGACAGACA
CCTCAGTTTTTCTGGATCAGGCATTGATGATGATGAAGATTTTATCTCCAGCACCATTTCAACCA
CACCACGGGCTTTTGACCACACAAAACAGAACCAGGACTGGACCCAGTGGAACCCAAGCCATTCA
AATCCGGAAGTGCTACTTCAGACAACCACAAGGATGACTGATGTAGACAGAAATGGCACCACTGC
TTATGAAGGAAACTGGAACCCAGAAGCACACCCTCCCCTCATTCACCATGAGCATCATGAGGAAG
AAGAGACCCCACATTCTACAAGCACAATCCAGGCAACTCCTAGTAGTACAACGGAAGAAACAGCT
ACCCAGAAGGAACAGTGGTTTGGCAACAGATGGCATGAGGGATATCGCCAAACACCCAAAGAAGA
CTCCCATTCGACAACAGGGACAGCTGCAGCCTCAGCTCATACCAGCCATCCAATGCAAGGAAGGA
CAACACCAAGCCCAGAGGACAGTTCCTGGACTGATTTCTTCAACCCAATCTCACACCCCATGGGA
CGAGGTCATCAAGCAGGAAGAAGGATGGATATGGACTCCAGTCATAGTATAACGCTTCAGCCTAC
TGCAAATCCAAACACAGGTTTGGTGGAAGATTTGGACAGGACAGGACCTCTTTCAATGACAACGC
AGCAGAGTAATTCTCAGAGCTTCTCTACATCACATGAAGGCTTGGAAGAAGATAAAGACCATCCA
ACAACTTCTACTCTGACATCAAGCAATAGGAATGATGTCACAGGTGGAAGAAGAGACCCAAATCA
TTCTGAAGGCTCAACTACTTTACTGGAAGGTTATACCTCTCATTACCCACACACGAAGGAAAGCA
GGACCTTCATCCCAGTGACCTCAGCTAAGACTGGGTCCTTTGGAGTTACTGCAGTTACTGTTGGA
GATTCCAACTCTAATGTCAATCGTTCCTTATCAGGAGACCAAGACACATTCCACCCCAGTGGGGG
GTCCCATACCACTCATGGATCTGAATCAGATGGACACTCACATGGGAGTCAAGAAGGTGGAGCAA
ACACAACCTCTGGTCCTATAAGGACACCCCAAATTCCAGAATGGCTGATCATCTTGGCATCCCTC
TTGGCCTTGGCTTTGATTCTTGCAGTTTGCATTGCAGTCAACAGTCGAAGAAGGTGTGGGCAGAA
GAAAAAGCTAGTGATCAACAGTGGCAATGGAGCTGTGGAGGACAGAAAGCCAAGTGGACTCAACG
GAGAGGCCAGCAAGTCTCAGGAAATGGTGCATTTGGTGAACAAGGAGTCGTCAGAAACTCCAGAC
CAGTTTATGACAGCTGATGAGACAAGGAACCTGCAGAATGTGGACATGAAGATTGGGGTGTAACA
CCTACACCATTATCTTGGAAAGAAACAACCGTTGGAAACATAACCATTACAGGGAGCTGGGACAC
TTAACAGATGCAATGTGCTACTGATTGTTTCATTGCGAATCTTTTTTAGCATAAAATTTTCTACT
CTTTTTGTTTTTTGTGTTTTGTTCTTTAAAGTCAGGTCCAATTTGTAAAAACAGCATTGCTTTCT
GAAATTAGGGCCCAATTAATAATCAGCAAGAATTTGATCGTTCCAGTTCCCACTTGGAGGCCTTT
CATCCCTCGGGTGTGCTATGGATGGCTTCTAACAAAAACTACACATATGTATTCCTGATCGCCAA
CCTTTCCCCCACCAGCTAAGGACATTTCCCAGGGTTAATAGGGCCTGGTCCCTGGGAGGAAATTT
GAATGGGTCCATTTTGCCCTTCCATAGCCTAATCCCTGGGCATTGCTTTCCACTGAGGTTGGGGG
TTGGGGTGTACTAGTTACACATCTTCAACAGACCCCCTCTAGAAATTTTTCAGATGCTTCTGGGA
GACACCCAAAGGGTGAAGCTATTTATCTGTAGTAAACTATTTATCTGTGTTTTTGAAATATTAAA
CCCTGGATCAGTCCTTTGATCAGTATAATTTTTTAAAGTTACTTTGTCAGAGGCACAAAAGGGTT
TAAACTGATTCATAATAAATATCTGTACTTCTTCGATCTTCACCTTTTGTGCTGTGATTCTTCAG
TTTCTAAACCAGCACTGTCTGGGTCCCTACAATGTATCAGGAAGAGCTGAGAATGGTAAGGAGAC
TCTTCTAAGTCTTCATCTCAGAGACCCTGAGTTCCCACTCAGACCCACTCAGCCAAATCTCATGG
AAGACCAAGGAGGGCAGCACTGTTTTTGTTTTTTGTTTTTTGTTTTTTTTTTTTGACACTGTCCA
AAGGTTTTCCATCCTGTCCTGGAATCAGAGTTGGAAGCTGAGGAGCTTCAGCCTCTTTTATGGTT
TAATGGCCACCTGTTCTCTCCTGTGAAAGGCTTTGCAAAGTCACATTAAGTTTGCATGACCTGTT
ATCCCTGGGGCCCTATTTCATAGAGGCTGGCCCTATTAGTGATTTCCAAAAACAATATGGAAGTG
CCTTTTGATGTCTTACAATAAGAGAAGAAGCCAATGGAAATGAAAGAGATTGGCAAAGGGGAAGG
ATGATGCCATGTAGATCCTGTTTGACATTTTTATGGCTGTATTTGTAAACTTAAACACACCAGTG
TCTGTTCTTGATGCAGTTGCTATTTAGGATGAGTTAAGTGCCTGGGGAGTCCCTCAAAAGGTTAA
AGGGATTCCCATCATTGGAATCTTATCACCAGATAGGCAAGTTTATGACCAAACAAGAGAGTACT
GGCTTTATCCTCTAACCTCATATTTTCTCCCACTTGGCAAGTCCTTTGTGGCATTTATTCATCAG
TCAGGGTGTCCGATTGGTCCTAGAACTTCCAAAGGCTGCTTGTCATAGAAGCCATTGCATCTATA
AAGCAACGGCTCCTGTTAAATGGTATCTCCTTTCTGAGGCTCCTACTAAAAGTCATTTGTTACCT
AAACTTATGTGCTTAACAGGCAATGCTTCTCAGACCACAAAGCAGAAAGAAGAAGAAAAGCTCCT
GACTAAATCAGGGCTGGGCTTAGACAGAGTTGATCTGTAGAATATCTTTAAAGGAGAGATGTCAA
CTTTCTGCACTATTCCCAGCCTCTGCTCCTCCCTGTCTACCCTCTCCCCTCCCTCTCTCCCTCCA
CTTCACCCCACAATCTTGAAAAACTTCCTTTCTCTTCTGTGAACATCATTGGCCAGATCCATTTT
CAGTGGTCTGGATTTCTTTTTATTTTCTTTTCAACTTGAAAGAAACTGGACATTAGGCCACTATG
TGTTGTTACTGCCACTAGTGTTCAAGTGCCTCTTGTTTTCCCAGAGATTTCCTGGGTCTGCCAGA
GGCCCAGACAGGCTCACTCAAGCTCTTTAACTGAAAAGCAACAAGCCACTCCAGGACAAGGTTCA
AAATGGTTACAACAGCCTCTACCTGTCGCCCCAGGGAGAAAGGGGTAGTGATACAAGTCTCATAG
CCAGAGATGGTTTTCCACTCCTTCTAGATATTCCCAAAAAGAGGCTGAGACAGGAGGTTATTTTC
AATTTTATTTTGGAATTAAATACTTTTTTCCCTTTATTACTGTTGTAGTCCCTCACTTGGATATA
CCTCTGTTTTCACGATAGAAATAAGGGAGGTCTAGAGCTTCTATTCCTTGGCCATTGTCAACGGA
GAGCTGGCCAAGTCTTCACAAACCCTTGCAACATTGCCTGAAGTTTATGGAATAAGATGTATTCT
CACTCCCTTGATCTCAAGGGCGTAACTCTGGAAGCACAGCTTGACTACACGTCATTTTTACCAAT
GATTTTCAGGTGACCTGGGCTAAGTCATTTAAACTGGGTCTTTATAAAAGTAAAAGGCCAACATT
TAATTATTTTGCAAAGCAACCTAAGAGCTAAAGATGTAATTTTTCTTGCAATTGTAAATCTTTTG
TGTCTCCTGAAGACTTCCCTTAAAATTAGCTCTGAGTGAAAAATCAAAAGAGACAAAAGACATCT
TCGAATCCATATTTCAAGCCTGGTAGAATTGGCTTTTCTAGCAGAACCTTTCCAAAAGTTTTATA
TTGAGATTCATAACAACACCAAGAATTGATTTTGTAGCCAACATTCATTCAATACTGTTATATCA
GAGGAGTAGGAGAGAGGAAACATTTGACTTATCTGGAAAAGCAAAATGTACTTAAGAATAAGAAT
AACATGGTCCATTCACCTTTATGTTATAGATATGTCTTTGTGTAAATCATTTGTTTTGAGTTTTC
AAAGAATAGCCCATTGTTCATTCTTGTGCTGTACAATGACCACTGTTATTGTTACTTTGACTTTT
CAGAGCACACCCTTCCTCTGGTTTTTGTATATTTATTGATGGATCAATAATAATGAGGAAAGCAT
GATATGTATATTGCTGAGTTGAAAGCACTTATTGGAAAATATTAAAAGGCTAACATTAAAAGACT
AAAGGAAACAGAAAAAAAAAAAAAAAAA

One particular non-limiting example of inhibitory nucleic acid for Tim-3 is CAAACCAGGGUAUUCU. Particular non-limiting examples of inhibitory nucleic acid for CD44 include a sequence having from about 8 to 30 bases in length, and is targeted to any of: nucleotides 5 through 22 of the 5′ untranslated region; nucleotides 1133 through 1563 of a 3′ untranslated region; or nucleotides 58 through 95, nucleotides 98 through 135, nucleotides 152 through 169, nucleotides 171 through 230, nucleotides 270 through 287, nucleotides 310 through 333, nucleotides 349 through 366, nucleotides 384 through 401, nucleotides 418 through 451, nucleotides 475 through 499, nucleotides 536 through 592, nucleotides 637 through 705, nucleotides 761 through 798, nucleotides 819 through 923, nucleotides 940 through 986, nucleotides 1021 through 1038, or nucleotides 1083 through 1100 of a coding region of human CD44, or a subsequence thereof.

As set forth herein, particular non-limiting example of agents include aptamers. Particular non-limiting examples of aptamers that bind to 4-1BB include a sequence CCCWCWAGX wherein X=G or C; W is an independently selected modified pyrimidine; and C is an independently selected modified cytidine; or sequence YCGGAWGZ wherein X=G or C; Y=C or G; Z=C or W; where W is an independently selected modified pyrimidine; and C is an independently selected modified cytidine. An additional particular non-limiting example of an aptamer that binds to 4-1BB includes:

GGGAGAGAGGAAGAGGGAUGGGCGACCGAACGUGCCCUUCAAAGCCGUUC
ACUAACCAGUGGCAUAACCCAGAGGUCGAUAGUACUGGAUCCCCCC.

For expression in cells, nucleic acid, if desired, may be inserted into a vector. Accordingly, invention compositions and methods further include nucleic acid sequences inserted into a vector. The term “vector” refers to a plasmid, virus or other vehicle known in the art that can be manipulated by insertion or incorporation of a nucleic acid. Such vectors can be used for genetic manipulation (i.e., “cloning vectors”) or can be used to transcribe or translate the inserted nucleic acid (i.e., “expression vectors”). A vector generally contains at least an origin of replication for propagation in a cell and a promoter. Control elements, including expression control elements as set forth herein, present within a vector are included to facilitate proper transcription and translation (e.g., splicing signal for introns, maintenance of the correct reading frame of the gene to permit in-frame translation of mRNA and, stop codons etc.).

Nucleic acids can be obtained using various standard cloning and chemical synthesis techniques. Purity of nucleic acids can be determined through sequencing, gel electrophoresis and the like. For example, nucleic acids can be isolated using hybridization as set forth herein or computer-based database screening techniques known in the art. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

The term “isolated,” when used as a modifier of a composition (e.g., 4-1BB, the galectin, 4-1BB ligand, Tim-3 or CD44 sequences, proteins, peptides, antibodies, subsequences, nucleic acids, inhibitory nucleic acid, antisense, etc.), means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane. The term “isolated” does not exclude alternative physical forms of the composition, such as fusions/chimeras, multimers/oligomers, modifications (e.g., phosphorylation, glycosylation, lipidation) or derivatized forms, or forms expressed in host cells produced by the hand of man.

An “isolated” composition (e.g., a 4-1BB, the galectin, 4-1BB ligand, Tim3 or CD44 sequence, proteins, peptides, antibody, subsequences, nucleic acids, inhibitory nucleic acid, antisense, etc.) can also be “substantially pure” or “purified” when free of most or all of the materials with which it typically associates with in nature. Thus, an isolated sequence that also is substantially pure or purified does not include polypeptides or nucleic acids present among millions of other sequences, such as antibodies of an antibody library or nucleic acids in a genomic or cDNA library, for example. Typically, purity can be at least about 50%, 60% or more by mass. The purity can also be about 70% or 80% or more, and can be greater, for example, 90% or more. Purity can be determined by any appropriate method, including, for example, UV spectroscopy, chromatography (e.g., HPLC, gas phase), gel electrophoresis and sequence analysis (nucleic acid and peptide), and is typically relative to the amount of impurities, which typically does not include inert substances, such as water.

A “substantially pure” or “purified” composition can be combined with one or more other molecules. Thus, “substantially pure” or “purified” does not exclude combinations of compositions, such as combinations of 4-1BB, a galectin, 4-1BB ligand, Tim-3 or CD44 sequences or antibodies, subsequences, and other antibodies, agents, drugs or therapies.

As used herein, the term “recombinant,” when used as a modifier of sequences such as antibodies, polypeptides and polynucleotides, means that the compositions have been manipulated (i.e., engineered) in a fashion that generally does not occur in nature (e.g., in vitro). A particular example of a recombinant polypeptide would be where a 4-1BB, a galectin, 4-1BB ligand, Tim-3 or CD44 polypeptide is expressed by a cell transfected with a polynucleotide encoding the 4-1BB, the galectin, 4-1BB ligand, Tim-3 or CD44 sequence. A particular example of a recombinant polynucleotide would be where a nucleic acid (e.g., genomic or cDNA) encoding 4-1BB, a galectin, 4-1BB ligand, Tim-3 or CD44 cloned into a plasmid, with or without 5′, 3′ or intron regions that the gene is normally contiguous with in the genome of the organism. Another example of a recombinant polynucleotide or polypeptide is a hybrid or fusion sequence, such as a chimeric antibody that binds to 4-1BB, a galectin, 4-1BB ligand, Tim-3 or CD44, or a chimeric 4-1BB, a galectin, 4-1BB ligand, Tim-3 or CD44 sequence comprising a second sequence, such as a heterologous functional domain.

The term “subsequence” or “fragment” means a portion of the full length molecule. A subsequence of a polypeptide sequence, such as a 4-1BB, a galectin, 4-1BB ligand, Tim-3 or CD44 sequence, has at least one amino acid less than a full length 4-1BB, a galectin, 4-1BB ligand, Tim3 or CD44 sequence (e.g. one or more internal or terminal amino acid deletions from either amino or carboxy-termini). Subsequences therefore can be any length up to one amino acid less than the full-length native molecule.

Subsequences can vary in size, for example, from a polypeptide as small as an epitope capable of binding an antibody (i.e., about five-eight amino acids) up to one amino acid less than the entire length of a reference sequence, such as 4-1BB, a galectin, 4-1BB ligand, Tim-3 or CD44. In various embodiments, a polypeptide subsequence (fragment) is characterized as including or consisting of a 4-1BB, a galectin, 4-1BB ligand, Tim-3 or CD44 sequence having at least about 1 to 5, 5 to 10, 10 to 20, 20 to 30, 30, to 50, 50 to 100, 100 to 150, 150 to 200, 200 to 300, or 300 to 400, 400-500, 500-600, or 600-700 fewer amino acids less than full length native 4-1BB, a galectin, 4-1BB ligand, Tim-3 or CD44 sequence.

Subsequences can also include or consist of one or more amino acid additions or deletions, wherein the subsequence does not comprise full length native/wild type 4-1BB, a galectin, 4-1BB ligand, Tim-3 or CD44 sequence. Accordingly, the total sequence length can be greater, for example, where a 4-1BB, a galectin, 4-1BB ligand, Tim-3 or CD44 subsequence is fused to or forms a chimera with another polypeptide sequence.

Invention agents, and methods and uses that include agents can be in various physical forms therein, such as a liquid or solid form. Invention agents, and methods and uses that include agents, can include any amount or dose of the agent, and the agent. In particular embodiments, an agent is in a concentration range of about 10 μg/ml to 100 mg/ml, or in a range of about 100 μg/ml to 1,000 mg/ml, or at a concentration of about 1 mg/ml. In further particular embodiments, an agent is in an amount of 10-1,000 milligrams, or an amount of 10-100 milligrams.

Methods and uses of the invention can be performed in vivo, such as in a subject, in vitro, ex vivo, in a cell, in solution, in solid phase or in silica. In particular embodiments, methods and uses comprise modulating binding of 4-1BB to Galectin-9 in a subject to modulate an immune response in the subject. In certain embodiments, the subject is a mammal, for example, a human.

In accordance with the invention, there are provided uses of agents for modulating an immune response, and methods of modulating an immune responses in a subject. In one embodiment, a use or method decreases, reduces, inhibits, suppresses, limits or controls an undesirable or aberrant immune response, disorder or disease, an inflammatory response, disorder or disease, inflammation, or an autoimmune response, disorder or disease, or an adverse symptom of an undesirable or aberrant immune response, disorder or disease, an inflammatory response, disorder or disease, inflammation, or an autoimmune response, disorder or disease. In another embodiment, a use or method increases, stimulates, enhances, promotes, agonizes, induces or activates an immune response, inflammatory response or inflammation, for example an anti-cancer immune response or inflammatory response or anti-pathogen immune response or inflammatory response, such as an anti-viral immune response.

Methods and uses of the invention include administering an agent that modulates binding of 4-1BB to a galectin to the subject. The agent in turn modulates the immune response in the subject.

In various aspects of methods and uses of the invention, a subject has or has had an adverse symptom of an undesirable or aberrant immune response, disorder or disease, an inflammatory response, disorder or disease, inflammation, or an autoimmune response, disorder or disease. In additional various aspects of methods and uses of the invention, a subject is in need of treatment for an undesirable or aberrant immune response, disorder or disease, an inflammatory response, disorder or disease, inflammation, or an autoimmune response, disorder or disease. In further various aspects of methods and uses of the invention, a subject is at risk for an undesirable or aberrant immune response, disorder or disease, an inflammatory response, disorder or disease, inflammation, or an autoimmune response, disorder or disease.

As used herein, an “undesirable immune response” or “aberrant immune response” refers to any immune response, activity or function that is greater or less than desired or physiologically normal, acute or chronic. An undesirable immune response, function or activity can be a normal response, function or activity. However, such responses are generally characterized as an undesirable or aberrant increased or inappropriate response, activity or function of the immune system. Thus, normal immune responses so long as they are undesirable, even if not considered aberrant, are included within the meaning of these terms. An undesirable immune response, function or activity can also be an abnormal response, function or activity. An abnormal (aberrant) immune response, function or activity deviates from normal.

One non-limiting example of an undesirable or aberrant immune response is where the immune response is hyper-responsive, such as in the case of an autoimmune disorder or disease. Another example of an undesirable or aberrant immune response is where an immune response leads to acute or chronic inflammatory response or inflammation in any tissue or organ.

Undesirable or aberrant immune responses, inflammatory responses, or inflammation are characterized by many different physiological adverse symptoms or complications, which can be humoral, cell-mediated or a combination thereof. Responses, disorders and diseases that can be treated in accordance with the invention include, but are not limited to, those that either directly or indirectly lead to or cause cell or tissue/organ damage in a subject. At the whole body, regional or local level, an immune response, inflammatory response, or inflammation can be characterized by swelling, pain, headache, fever, nausea, skeletal joint stiffness or lack of mobility, rash, redness or other discoloration. At the cellular level, an immune response, inflammatory response, or inflammation can be characterized by one or more of T cell activation and/or differentiation, cell infiltration of the region, production of antibodies, production of cytokines, lymphokines, chemokines, interferons and interleukins, cell growth and maturation factors (e.g., proliferation and differentiation factors), cell accumulation or migration and cell, tissue or organ damage. Thus, methods and uses of the invention include treatment of and an ameliorative effect upon any such physiological symptoms or cellular or biological responses characteristic of immune responses, inflammatory response, or inflammation.

Autoimmune responses, disorders and diseases are generally characterized as an undesirable or aberrant response, activity or function of the immune system characterized by increased or undesirable humoral or cell-mediated immune responsiveness or memory, or decreased or insufficient tolerance to self-antigens. Autoimmune responses, disorders and diseases that may be treated in accordance with the invention include but are not limited to responses, disorders and diseases that cause cell or tissue/organ damage in the subject. The terms “immune disorder” and “immune disease” mean, an immune function or activity which is characterized by different physiological symptoms or abnormalities, depending upon the disorder or disease.

In particular embodiments, a method or use decreases, reduces, inhibits, suppresses, limits or controls an undesirable or aberrant immune response, disorder or disease, inflammatory response, disorder or disease, or inflammation, in a subject. In additional particular embodiments, a method or use decreases, reduces, inhibits, suppresses, limits or controls an autoimmune response, disorder or disease in a subject. In further particular embodiments, a method or use decreases, reduces, inhibits, suppresses, limits or controls an adverse symptom of the undesirable or aberrant immune response, disorder or disease, inflammatory response, disorder or disease, inflammation, or an autoimmune response, disorder or disease.

In additional particular embodiments, methods and uses according to the invention can result in a reduction in occurrence, frequency, severity, progression, or duration of a symptom of the condition (e.g., undesirable or aberrant immune response, disorder or disease, inflammatory response, disorder or disease, inflammation, or an autoimmune response, disorder or disease). For example, methods of the invention can protect against or decrease, reduce, inhibit, suppress, limit or control progression, severity, frequency, duration or probability of an adverse symptom of the undesirable or aberrant undesirable or aberrant immune response, disorder or disease, inflammatory response, disorder or disease, inflammation, or an autoimmune response, disorder or disease.

Examples of adverse symptoms of an undesirable or aberrant immune response, disorder or disease, inflammatory response, disorder or disease, inflammation, or an autoimmune response, disorder or disease include swelling, pain, rash, discoloration, headache, fever, nausea, diarrhea, bloat, lethargy, skeletal joint stiffness, reduced muscle or limb mobility or of the subject, paralysis, a sensory impairment, such as vision or tissue or cell damage. Examples of adverse symptoms occur in particular tissues, or organs, or regions or areas of the body, such as in skin, epidermal or mucosal tissue, gut, gastrointestinal, bowel, genito-urinary tract, pancreas, thymus, lung, liver, kidney, muscle, central or peripheral nerves, spleen, skin, a skeletal joint (e.g., knee, ankle, hip, shoulder, wrist, finger, toe, or elbow), blood or lymphatic vessel, or a cardio-pulmonary tissue or organ. Additional examples of adverse symptoms of an autoimmune response, disorder or disease include T cell production, survival, proliferation, activation or differentiation, and/or production of auto-antibodies, or pro-inflammatory cytokines or chemokines (e.g., TNF-alpha, IL-6, etc.).

Specific non-limiting examples of undesirable or aberrant undesirable or aberrant immune response, disorder or disease, inflammatory response, disorder or disease, inflammation, or an autoimmune response, disorder or disease treatable in accordance with the invention include: rheumatoid arthritis, juvenile rheumatoid arthritis, osteoarthritis, psoriatic arthritis, multiple sclerosis (MS), encephalomyelitis, myasthenia gravis, systemic lupus erythematosus (SLE), asthma, allergic asthma, autoimmune thyroiditis, atopic dermatitis, eczematous dermatitis, psoriasis, Sjögren's Syndrome, Crohn's disease, aphthous ulcer, iritis, conjunctivitis, keratoconjunctivitis, ulcerative colitis (UC), inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus (IDDM, type I diabetes), insulin-resistant diabetes mellitus (type 11 diabetes), immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common γ chain (γc) deficiency, Janus-associated kinase 3 (JAK3) deficiency and reticular dysgenesis; primary T cell immunodeficiency such as DiGcorge syndrome, Nude syndrome, T cell receptor deficiency, MHC class II deficiency, T AP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (γ5/14.1) deficiency, Hyper-IgM syndrome: X-linked (CD40 ligand deficiency) or non-X-Iinked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon γ receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1A/SAP deficiency), hyper IgE syndrome and Graft vs. Host Disease (GVHD).

In methods and uses of the invention, an agent (e.g., a 4-1BB, 4-1BBL, a galectin (e.g., Galectin-9), Tim-3, or CD44 antibody, protein subsequence, nucleic acid, inhibitory nucleic acid, etc.) can be administered prior to, substantially contemporaneously with or following an undesirable or aberrant immune response, disorder or disease, inflammatory response, disorder or disease, inflammation, or an autoimmune response, disorder or disease, or one or more adverse symptoms, disorders, illnesses, pathologies, diseases, or complications caused by or associated with the foregoing. Thus, methods and uses of the invention may be practiced prior to (i.e. prophylaxis), concurrently with or after evidence of the response, disorder or disease begins, or one or more adverse symptoms, disorders, illnesses, pathologies, diseases, or complications caused by or associated with the undesirable or aberrant immune response, disorder or disease, inflammatory response, disorder or disease, inflammation, or an autoimmune response, disorder or disease. Administering an agent (e.g., a 4-1BB, 4-1BBL, a galectin (e.g., Galectin-9), Tim-3, or CD44 antibody, protein subsequence, nucleic acid, inhibitory nucleic acid, etc.) sequence prior to, concurrently with or immediately following development of an adverse symptom may decrease, reduce, inhibit, suppress, limit or control the occurrence, frequency, severity, progression, or duration of one or more adverse symptoms, disorders, illnesses, pathologies, diseases, or complications caused by or associated with the undesirable or aberrant immune response, disorder or disease, inflammatory response, disorder or disease, inflammation, or an autoimmune response, disorder or disease.

In particular embodiments, a method or use increases, stimulates, enhances, promotes, agonizes, induces or activates an immune response, inflammatory response or inflammation, for example an anti-cancer immune response or inflammatory response or anti-pathogen immune response or inflammatory response, such as an anti-viral immune response.

Specific non-limiting examples of an immune response, inflammatory response or inflammation to be increased, stimulated, enhanced, promoted, agonized, induced or activated are an immune response, inflammatory response or inflammation associated with or caused by a cancer cell, tumor, viral infection or bacterial infection.

In methods and uses of the invention, an agent (e.g., a 4-1BB, 4-1BBL, a galectin (e.g., Galectin-9), Tim-3, or CD44 antibody, protein subsequence, nucleic acid, inhibitory nucleic acid, etc.) can be administered prior to, substantially contemporaneously with or following an of an immune response, inflammatory response or inflammation to be increased, stimulated, enhanced, promoted, agonized, induced or activated. Thus, methods and uses of the invention may be practiced prior to (i.e. prophylaxis), concurrently with or after evidence of the response or inflammation begins. Administering an agent (e.g., a 4-1BB, 4-1BBL, a galectin (e.g., Galectin-9), Tim-3, or CD44 antibody, protein subsequence, nucleic acid, inhibitory nucleic acid, etc.) sequence prior to, concurrently with or immediately following development of an immune response, inflammatory response or inflammation may increase, stimulate, enhance, promote, agonize, induce or activated the occurrence, frequency, severity, progression, or duration of the immune response, inflammatory response or inflammation, for example an anti-cancer or anti-pathogen immune response.

The invention provides combination compositions, methods and uses, such as an agent (e.g., a 4-1BB, 4-1BBL, a galectin (e.g., Galectin-9), Tim-3, or CD44 antibody, protein subsequence, nucleic acid, inhibitory nucleic acid, etc.) and a second agent or drug. In one embodiment, a composition, method or use includes an agent (e.g., a 4-1BB, 4-1BBL, a galectin (e.g., Galectin-9), Tim-3, or CD44 antibody, protein subsequence, nucleic acid, inhibitory nucleic acid, etc.) and an anti-inflammatory agent or drug. Such agents and drugs useful in combinations and in methods and uses of the invention include drugs and agents for treatment of an undesirable or aberrant immune response, disorder or disease, inflammatory response, disorder or disease, inflammation, or an autoimmune response, disorder or disease.

An agent (e.g., a 4-1BB, 4-1BBL, a galectin (e.g., Galectin-9), Tim-3, or CD44 antibody, protein subsequence, nucleic acid, inhibitory nucleic acid, etc.) or a composition thereof can be formulated and/or administered in combination with a second agent, drug or treatment, such as an immunosuppressive, anti-inflammatory, or palliative agent, drug or treatment. According to the invention, an agent (e.g., a 4-1BB, 4-1BBL, a galectin (e.g., Galectin-9), Tim-3, or CD44 antibody, protein subsequence, nucleic acid, inhibitory nucleic acid, etc.) or a composition thereof can be formulated as a combination and/or administered prior to, substantially contemporaneously with or following administering a second agent, drug or treatment, such as an immunosuppressive, anti-inflammatory, or palliative agent, drug or treatment.

Non-limiting examples of second agents and drugs include anti-inflammatory agents, such as steroidal and non-steroidal anti-inflammatory drugs (NSAIDs) to limit or control inflammatory symptoms. Second agents and drugs also include immunosuppressive corticosteroids (steroid receptor agonists) such as budesonide, prednisone, flunisolide; anti-inflammatory agents such as flunisolide hydrofluoroalkane, estrogen, progesterone, dexamethasone and loteprednol; beta-agonists (e.g., short or long-acting) such as bambuterol, formoterol, salmeterol, albuterol; anticholinergics such as ipratropium bromide, oxitropium bromide, cromolyn and calcium-channel blocking agents; antihistamines such as terfenadine, astemizole, hydroxyzine, chlorpheniramine, tripelennamine, cetirizine, desloratadine, mizolastine, fexofenadine, olopatadine hydrochloride, norastemizole, levocetirizine, levocabastine, azelastine, ebastine and loratadine; antileukotrienes (e.g., anti-cysteinyl leukotrienes (CysLTs)) such as oxatomide, montelukast, zafirlukast and zileuton; phosphodiesterase inhibitors (e.g., PDE4 subtype) such as ibudilast, cilomilast, BAY 19-8004, theophylline (e.g., sustained-release) and other xanthine derivatives (e.g., doxofylline); thromboxane antagonists such as seratrodast, ozagrel hydrochloride and ramatroban; prostaglandin antagonists such as COX-1 and COX-2 inhibitors (e.g., celecoxib and rofecoxib), aspirin; and potassium channel openers. Additional non-limiting examples of classes of other agents and drugs include anti-inflammatory agents that are immunomodulatory therapies, such as pro-inflammatory cytokine antagonists, such as TNFα antagonists (e.g. etanercept, aka Enbrel™) and the anti-IL-6 receptor tocilizumab; immune cell antagonists, such as the B cell depleting agent rituximab and the T cell costimulation blocker abatacept, which have been used to treat rheumatoid arthritis, and antibodies that bind to cytokines, such as anti-IgE (e.g., rhuMAb-E25 omalizumab), and anti-TNFα, IFNγ, IL-1, IL-2, IL-5, IL-6, IL-9, IL-13, IL-16, and growth factors such as granulocyte/macrophage colony-stimulating factor.

As disclosed herein, agents, methods and uses, such as treatment methods and uses, can provide a detectable or measurable therapeutic benefit or improvement to a subject. A therapeutic benefit or improvement is any measurable or detectable, objective or subjective, transient, temporary, or longer-term benefit to the subject or improvement in the response, disorder or disease, or one or more adverse symptoms, disorders, illnesses, pathologies, diseases, or complications caused by or associated with the undesirable or aberrant immune response, disorder or disease, inflammatory response, disorder or disease, inflammation, or an autoimmune response, disorder or disease. Therapeutic benefits and improvements include, but are not limited to, decreasing, reducing, inhibiting, suppressing, limiting or controlling the occurrence, frequency, severity, progression, or duration of an adverse symptom of undesirable or aberrant immune response, disorder or disease, inflammatory response, disorder or disease, inflammation, or an autoimmune response, disorder or disease. Therapeutic benefits and improvements also include, but are not limited to, decreasing, reducing, inhibiting, suppressing, limiting or controlling amounts or activity of T cells, auto-antibodies, pro-inflammatory cytokines or chemokines. Therapeutic benefits and improvements also include, but are not limited to, increasing, stimulating, enhancing, promoting, agonizing, inducing or activating an immune response, inflammatory response or inflammation, for example an anti-cancer or anti-pathogen immune response or inflammatory response. Agents, methods and uses of the invention therefore include providing a therapeutic benefit or improvement to a subject.

Agents (e.g., a 4-1BB, 4-1BBL, a galectin (e.g., Galectin-9), Tim-3, or CD44 antibody, protein subsequence, nucleic acid, inhibitory nucleic acid, etc.), methods and uses of the invention, can be administered in a sufficient or effective amount to a subject in need thereof. An “effective amount” or “sufficient amount” refers to an amount that provides, or is predicted to provide, in single or multiple doses, alone or in combination, with one or more other compositions (therapeutic agents such as a drug), treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured).

The doses of an “effective amount” or “sufficient amount” for treatment (e.g., to ameliorate or to provide a therapeutic benefit or improvement) typically are effective to ameliorate a response, disorder or disease, or one, multiple or all adverse symptoms, consequences or complications of the response, disorder or disease, one or more adverse symptoms, disorders, illnesses, pathologies, diseases, or complications, for example, caused by or associated with an undesirable or aberrant immune response, disorder or disease, inflammatory response, disorder or disease, inflammation, or an autoimmune response, disorder or disease, to a measurable extent, although decreasing, reducing, inhibiting, suppressing, limiting or controlling progression or worsening of the undesirable or aberrant immune response, disorder or disease, inflammatory response, disorder or disease, inflammation, or an autoimmune response, disorder or disease, or an adverse symptom thereof, is a satisfactory outcome.

An effective amount or a sufficient amount can but need not be provided in a single dose or administration, may require multiple doses or administrations, and, can but need not be, administered alone or in combination with another composition (e.g., agent), treatment, protocol or therapeutic regimen. For example, the amount may be proportionally increased as indicated by the need of the subject, type, status and severity of the response, disorder, or disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use or frequency or amount of another treatment, therapeutic regimen or protocol.

An effective amount or a sufficient amount need not be effective in each and every subject treated, prophylactically or therapeutically, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use. Thus, appropriate amounts will depend upon the condition treated, the therapeutic effect desired, as well as the individual subject (e.g., the bioavailability within the subject, gender, age, etc.).

The term “ameliorate” means a detectable or measurable improvement in a subject's condition or an underlying cellular response. A detectable or measurable improvement includes a subjective or objective decrease, reduction, inhibition, suppression, limit or control in the occurrence, frequency, severity, progression, or duration of the response, disorder or disease, such as an undesirable or aberrant immune response, disorder or disease, inflammatory response, disorder or disease, inflammation, or an autoimmune response, disorder or disease, or one or more adverse symptoms, disorders, illnesses, pathologies, diseases, or complications caused by or associated with an undesirable or aberrant immune response, disorder or disease, inflammatory response, disorder or disease, inflammation, or an autoimmune response, disorder or disease, or an improvement in an underlying cause or a consequence of the undesirable or aberrant immune response, disorder or disease, inflammatory response, disorder or disease, inflammation, or an autoimmune response, disorder or disease, or a reversal of the response, disorder or disease such as undesirable or aberrant immune response, disorder or disease, inflammatory response, disorder or disease, inflammation, or an autoimmune response, disorder or disease. Such improvements can also occur at the cellular level.

Thus, a successful treatment outcome can lead to a “therapeutic effect,” or “benefit” of decreasing, reducing, inhibiting, suppressing, limiting, controlling or preventing the occurrence, frequency, severity, progression, or duration of an undesirable or aberrant immune response, disorder or disease, inflammatory response, disorder or disease, inflammation, or an autoimmune response, disorder or disease, or one or more adverse symptoms or underlying causes or consequences of the undesirable or aberrant immune response, disorder or disease, inflammatory response, disorder or disease, inflammation, or an autoimmune response, disorder or disease in a subject. Treatment methods affecting one or more underlying causes of the response, disorder or disease or adverse symptom are therefore considered to be beneficial. A decrease or reduction in worsening, such as stabilizing an undesirable or aberrant immune response, disorder or disease, inflammatory response, disorder or disease, inflammation, or an autoimmune response, disorder or disease, or an adverse symptom thereof, is also a successful treatment outcome.

A therapeutic benefit or improvement therefore need not be complete ablation of the undesirable or aberrant immune response, disorder or disease, inflammatory response, disorder or disease, inflammation, or an autoimmune response, disorder or disease, or any particular one, most or all adverse symptoms, complications, consequences or underlying causes associated with the undesirable or aberrant immune response, disorder or disease, inflammatory response, disorder or disease, inflammation, or an autoimmune response, disorder or disease. Thus, a satisfactory endpoint is achieved when there is an incremental improvement in a subject's response, disorder or disease, or a partial decrease, reduction, inhibition, suppression, limit, control or prevention in the occurrence, frequency, severity, progression, or duration, or inhibition or reversal, of the response, disorder or disease (e.g., stabilizing one or more symptoms or complications), such as undesirable or aberrant immune response, disorder or disease, inflammatory response, disorder or disease, inflammation, or an autoimmune response, disorder or disease, or one or more adverse symptoms, disorders, illnesses, pathologies, diseases, or complications caused by or associated with an undesirable or aberrant immune response, disorder or disease, inflammatory response, disorder or disease, inflammation, or an autoimmune response, disorder or disease, over a short or long duration of time (hours, days, weeks, months, etc.).

Effectiveness of a method or use, such as a treatment that provides a potential therapeutic benefit or improvement of a response, disorder or disease, such as undesirable or aberrant immune response, disorder or disease, inflammatory response, disorder or disease, inflammation, or an autoimmune response, disorder or disease, can be ascertained by various methods. Such methods include, for example, scores measuring inflammation, swelling, pain, rash, headache, fever, nausea, diarrhea, bloat, lethargy, skeletal joint stiffness, lack of mobility, rash, or tissue or cell damage. Measuring T cell activation and/or differentiation, cell infiltration of a region, cell accumulation or migration to a region, production of antibodies, cytokines, lymphokines, chemokines, interferons and interleukins, cell growth and maturation factors using various immunological assays, such as ELISA. Determining the degree of cell, tissue or organ damage can be ascertained by CT scanning, MRI, ultrasound, molecular contrast imaging, or molecular ultrasound contrast imaging. For gastrointestinal tract, inflammation can be assessed by endoscopy (colonoscopy, gastroscopy, ERCP), for example. For inflammation of the central nervous system (CNS), cells and cytokines in spinal tap reflect inflammation, for example. CNS inflammation (Multiple sclerosis, Parkinson's, Alzheimer's) may be reflected in the corresponding clinical function scores known in the art, for example. Peripheral nerve inflammation can include functional assessment (motor and sensor), for example.

The term “subject” refers to animals, typically mammalian animals, such as humans, non human primates (e.g., apes, gibbons, chimpanzees, orangutans, macaques), domestic animals (e.g., dogs and cats), farm animals (e.g., horses, cows, goats, sheep, pigs) and experimental animals (e.g., mouse, rat, rabbit, guinea pig). Subjects include animal disease models, for example, animal models of undesirable or aberrant immune response, disorder or disease, inflammatory response, disorder or disease, inflammation, or an autoimmune response, disorder or disease (e.g., CIA, BXSB, EAE and SCID mice), for in vivo analysis of an agent of the invention.

Subjects appropriate for treatment include those having an undesirable or aberrant immune response, disorder or disease, inflammatory response, disorder or disease, inflammation, or an autoimmune response, disorder or disease, those undergoing treatment for an undesirable or aberrant immune response, disorder or disease, inflammatory response, disorder or disease, inflammation, or an autoimmune response, disorder or disease, as well as those who have undergone treatment or therapy for an undesirable or aberrant immune response, disorder or disease, inflammatory response, disorder or disease, inflammation, or an autoimmune response, disorder or disease, including subjects where the undesirable or aberrant immune response, disorder or disease, inflammatory response, disorder or disease, inflammation, or an autoimmune response, disorder or disease, is in remission.

Subjects also include those that are at increased risk of an undesirable or aberrant immune response, disorder or disease, inflammatory response, disorder or disease, inflammation, or an autoimmune response, disorder or disease. A candidate subject, for example, has an undesirable or aberrant immune response, disorder or disease, inflammatory response, disorder or disease, inflammation, or an autoimmune response, disorder or disease, or is being treated with a therapy or drug for an undesirable or aberrant immune response, disorder or disease, inflammatory response, disorder or disease, inflammation, or an autoimmune response, disorder or disease. Candidate subjects also include subjects that would benefit from or are in need of treatment for an undesirable or aberrant immune response, disorder or disease, inflammatory response, disorder or disease, inflammation, or an autoimmune response, disorder or disease.

“At risk” subjects typically have increased risk factors for an undesirable or aberrant immune response, disorder or disease, inflammatory response, disorder or disease, inflammation, or an autoimmune response, disorder or disease. Particular subjects at risk include those that have had an undesirable or aberrant immune response, disorder or disease, inflammatory response, disorder or disease, inflammation, or an autoimmune response, disorder or disease. Particular subjects at risk also include those prescribed a treatment or therapy of an undesirable or aberrant immune response, disorder or disease, inflammatory response, disorder or disease, inflammation, or an autoimmune response, disorder or disease. At risk subjects also include those with risk factors include family history (e.g., genetic predisposition), gender, lifestyle (diet, smoking), occupation (medical and clinical personnel, agricultural and livestock workers), environmental factors (allergen exposure), etc.

As set forth herein, agents (e.g., a 4-1BB, 4-1BBL, a galectin (e.g., Galectin-9), Tim-3, or CD44 antibody, protein subsequence, nucleic acid, inhibitory nucleic acid, etc.) and compositions thereof may be contacted or provided in vitro, ex vivo or administered or delivered in vivo in various doses and amounts, and frequencies. For example, an agent (e.g., a 4-1BB, 4-1BBL, a galectin (e.g., Galectin-9), Tim-3, or CD44 antibody, protein subsequence, nucleic acid, inhibitory nucleic acid, etc.) can be administered or delivered to provide the intended effect, as a single or as multiple dosages, for example, in an effective or sufficient amount.

Exemplary doses range from about 25-250, 250-500, 500-1000, 1000-2500, 2500-5000, 5000-25,000, or 5000-50,000 pg/kg; from about 50-500, 500-5000, 5000-25,000 or 25,000-50,000 ng/kg; from about 50-500, 500-5000, 5000-25,000 or 25,000-50,000 pg/kg; and from about 25-250, 250-500, 500-1000, 1000-2500, 2500-5000, 5000-25,000, or 5000-50,000 mg/kg, on consecutive days, alternating days or intermittently (bi-weekely, weekly, 2, 3 or 4 times/month, monthly, etc.). In certain embodiments an agent may be provided in a range of about 100 μg/ml to 1,000 mg/ml. In further particular embodiments the agent may be administered in a amount of 1-10,000, 1-1,000, 10-100, or 30-75 milligrams to the subject.

Single or multiple (e.g., 2, 3, 4, 5, 6, 7, 8, 9, 10, or more times) administrations or doses can be administered on the same or consecutive days, alternating days or intermittently. For example, an agent (e.g., a 4-1BB, 4-1BBL, a galectin (e.g., Galectin-9), Tim-3, or CD44 antibody, protein subsequence, nucleic acid, inhibitory nucleic acid, etc.) or a composition thereof can be administered one, two, three, four or more times daily, on alternating days, bi-weekly, weekly, monthly, bi-monthly, or annually.

Agents (e.g., a 4-1BB, 4-1BBL, a galectin (e.g., Galectin-9), Tim-3, or CD44 antibody, protein subsequence, nucleic acid, inhibitory nucleic acid, etc.) and compositions thereof can be administered for any appropriate duration, for example, for period of 1 hour, or less, e.g., 30 minutes or less, 15 minutes or less, 5 minutes or less, or 1 minute or less. In particular embodiments, the agent may be administered for a period of 30 minutes or less, for a period of 15 minutes or less, or for a period of 5 minutes or less, or for a period of 1 minute or less.

An agent (e.g., a 4-1BB, 4-1BBL, a galectin (e.g., Galectin-9), Tim-3, or CD44 antibody, protein subsequence, nucleic acid, inhibitory nucleic acid, etc.) that modulates binding of 4-1BB to a galectin may be provided or administered by any suitable method known in the art. In various embodiments, an agent may be administered by injection, infusion, catheter, cnctna, intravenously, intraatterially, orally, intramuscularly, intraperitoneally, intradermally, subcutaneously, intracavity, intrarectally, intracranially, topically, transdermally, optically, parenterally, or transmucosally.

In particular embodiments, an agent (e.g., a 4-1BB, 4-1BBL, a galectin (e.g., Galectin-9), Tim-3, or CD44 antibody, protein subsequence, nucleic acid, inhibitory nucleic acid, etc.) may be administered in a pharmaceutically acceptable carrier, for example saline.

Agents can be administered to a subject and methods and uses may be practiced prior to, substantially contemporaneously with, or within about 1-60 minutes, hours (e.g., within 1, 2, 3, 4, 5, 6, 8, 12, 24 hours), or days of a symptom or onset of an undesirable or aberrant immune response, disorder or disease, inflammatory response, disorder or disease, inflammation, or an autoimmune response, disorder or disease.

An agent (e.g., a 4-1BB, 4-1BBL, a galectin (e.g., Galectin-9), Tim-3, or CD44 antibody, protein subsequence, nucleic acid, inhibitory nucleic acid, etc.) can be administered and methods and uses may be practiced via systemic, regional or local delivery or administration, by any route. For example, an agent (e.g., a 4-1BB, 4-1BBL, a galectin (e.g., Galectin-9), Tim-3, or CD44 antibody, protein subsequence, nucleic acid, inhibitory nucleic acid, etc.) or composition thereof may be administered systemically, regionally or locally, via injection, infusion, orally (e.g., ingestion or inhalation), topically, intravenously, intraarterially, intramuscularly, intraperitoneally, intradermally, subcutaneously, intracavity, intracranially, transdermally (topical), parenterally, e.g. transmucosally or intrarectally (enema) catheter, optically. Agents (e.g., a 4-1BB, 4-1BBL, a galectin (e.g., Galectin-9), Tim-3, or CD44 antibody, protein subsequence, nucleic acid, inhibitory nucleic acid, etc.), methods and uses of the invention including pharmaceutical formulations can be administered via a (micro)encapsulated delivery system or packaged into an implant for administration.

Invention compositions, methods and uses include pharmaceutical compositions, which refer to “pharmaceutically acceptable” and “physiologically acceptable” carriers, diluents or excipients. As used herein, the term “pharmaceutically acceptable” and “physiologically acceptable,” when referring to carriers, diluents or excipients includes solvents (aqueous or non-aqueous), detergents, solutions, emulsions, dispersion media, coatings, isotonic and absorption promoting or delaying agents, compatible with pharmaceutical administration and with the other components of the formulation, and can be contained in a tablet (coated or uncoated), capsule (hard or soft), microbead, emulsion, powder, granule, crystal, suspension, syrup or elixir.

In various embodiments, a pharmaceutical composition includes an agent. In a particular aspect, an agent includes or consists of a 4-1BB, 4-1BBL, a galectin (e.g., Galectin-9), Tim-3, or CD44 antibody, protein subsequence, nucleic acid, inhibitory nucleic acid, etc.

Pharmaceutical compositions can be formulated to be compatible with a particular route of administration. Compositions for parenteral, intradermal, or subcutaneous administration can include a sterile diluent, such as water, saline, fixed oils, polyethylene glycols, glycerine, propylene glycol or other synthetic solvents. The preparation may contain one or more preservatives to prevent microorganism growth (e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose).

Pharmaceutical compositions for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and polyetheylene glycol), and suitable mixtures thereof. Fluidity can be maintained, for example, by the use of a coating such as lecithin, or by the use of surfactants. Antibacterial and antifungal agents include, for example, parabens, chlorobutanol, phenol, ascorbic acid and thimerosal. Including an agent that delays absorption, for example, aluminum monostearate and gelatin, can prolong absorption of injectable compositions.

For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays, inhalation devices (e.g., aspirators) or suppositories. For transdermal administration, the active compounds are formulated into ointments, salves, gels, creams or patches.

Additional pharmaceutical formulations and delivery systems are known in the art and are applicable in the methods of the invention (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms, Technonic Publishing Co., Inc., Lancaster, Pa., (1993); and Poznansky, et al., Drug Delivery Systems, R. L. Juliano, ed., Oxford, N.Y. (1980), pp. 253-315).

The compositions, methods and uses in accordance with the invention, including a 4-1BB, 4-1BBL, a galectin (e.g., Galectin-9), Tim-3, or CD44 antibody, protein subsequence, nucleic acid, inhibitory nucleic acid, sequences, subsequences, variants and modified forms, polymorphisms, treatments, therapies, combinations, agents, drugs and pharmaceutical formulations can be packaged in dosage unit form for ease of administration and uniformity of dosage. “Dosage unit form” as used herein refers to physically discrete units suited as unitary dosages treatment; each unit contains a quantity of the composition in association with the carrier, excipient, diluent, or vehicle calculated to produce the desired treatment or therapeutic (e.g., beneficial) effect. The unit dosage forms will depend on a variety of factors including, but not necessarily limited to, the particular composition employed, the effect to be achieved, and the pharmacodynamics and pharmacogenomics of the subject to be treated.

The invention provides kits including an agent (e.g., a 4-1BB, 4-1BBL, a galectin (e.g., Galectin-9), Tim-3, or CD44 antibody, protein subsequence, nucleic acid, inhibitory nucleic acid, etc.), combination compositions and pharmaceutical formulations thereof, packaged into suitable packaging material. Kits can be used in various in vitro, ex vivo and in vivo methods and uses, for example a treatment method or use as disclosed herein.

A kit typically includes a label or packaging insert including a description of the components or instructions for use in vitro, in vivo, or ex vivo, of the components therein. A kit can contain a collection of such components, e.g., an agent (e.g., a 4-1BB, 4-1BBL, a galectin (e.g., Galectin-9), Tim-3, or CD44 antibody, protein subsequence, nucleic acid, inhibitory nucleic acid, etc.), alone, or in combination with another therapeutically useful composition (e.g., an immune modulatory drug).

The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits of the invention can include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk (e.g., hard disk), optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer information, lot numbers, manufacturer location and date.

Labels or inserts can include information on a condition, disorder, disease or symptom for which a kit component may be used. Labels or inserts can include instructions for the clinician or for a subject for using one or more of the kit components in a method, treatment protocol or therapeutic regimen. Instructions can include dosage amounts, frequency or duration, and instructions for practicing any of the methods and uses, treatment protocols or therapeutic regimes set forth herein. Exemplary instructions include, instructions for treating an undesirable or aberrant immune response, disorder or disease, inflammatory response, disorder or disease, inflammation, or an autoimmune response, disorder or disease. Kits of the invention therefore can additionally include labels or instructions for practicing any of the methods and uses of the invention described herein.

Labels or inserts can include information on any benefit that a component may provide, such as a prophylactic or therapeutic benefit. Labels or inserts can include information on potential adverse side effects, such as warnings to the subject or clinician regarding situations where it would not be appropriate to use a particular composition. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Invention kits can additionally include other components. Each component of the kit can be enclosed within an individual container and all of the various containers can be within a single package. Invention kits can be designed for cold storage. Invention kits can further be designed to contain an agent (e.g., a 4-1BB, 4-1BBL, a galectin (e.g., Galectin-9), Tim-3, or CD44 antibody, protein subsequence, nucleic acid, inhibitory nucleic acid, etc.), or combination composition or pharmaceutical composition.

The invention provides cell-free (e.g., in solution, in solid phase) and cell-based (e.g., in vitro or in vivo) methods of screening for, detecting and identifying agents that modulate binding (interaction) between 4-1BB and a galectin (e.g., Galectin-9), and methods of screening, detecting and identifying agents that modulate an undesirable or aberrant immune response, disorder or disease, inflammatory response, disorder or disease, inflammation, or an autoimmune response, disorder or disease. The methods can be performed in solution, in solid phase, in silica, in vitro, in a cell, and in vivo.

In one embodiment, a method of screening for an agent includes a) contacting 4-1BB with a galectin (e.g., Galectin-9) in the presence of a test agent under conditions allowing binding of 4-1BB to the galectin (e.g., Galectin-9); and b) determining if the test agent modulates binding of 4-1BB to the galectin (e.g., Galectin-9). An agent that modulates (alters, affects) binding of 4-1BB to the galectin (e.g., Galectin-9) indicates that the test agent is an agent that modulates binding of 4-1BB with the galectin (e.g., Galectin-9).

In one embodiment, a method of identifying an agent includes a) contacting 4-IBB with the galectin (e.g., Galectin-9) in the presence a test agent under conditions allowing binding of 4-1BB to the galectin (e.g., Galectin-9); and b) determining if the test agent modulates binding of 4-1BB to the galectin (e.g., Galectin-9). A test agent that modulates binding of 4-1BB to the galectin (e.g., Galectin-9) indicates that the test agent is an agent that modulates binding of 4-1BB to the galectin (e.g., Galectin-9).

In various aspects, a method includes screening for or identifying an agent for decreasing, reducing, inhibiting, suppressing, limiting or controlling undesirable or aberrant immune response, disorder or disease, an inflammatory response, disorder or disease, inflammation, or an autoimmune response, disorder or disease. In other aspects, a method is for screening for or identifying an agent for the treatment of a tumor or cancer.

In a further embodiment, a method of identifying a candidate agent for modulating (e.g., decreasing, reducing, inhibiting, suppressing, limiting or controlling) an undesirable or aberrant immune response, disorder or disease, inflammatory response, disorder or disease, inflammation, or an autoimmune response, disorder or disease, is provided. Such a test agent identified as reducing or inhibiting binding, the test agent is a candidate agent for decreasing, reducing, inhibiting, suppressing, limiting or controlling an undesirable or aberrant immune response, disorder or disease, inflammatory response, disorder or disease, inflammation, or an autoimmune response, disorder or disease.

The terms “determining,” “assaying” and “measuring” and grammatical variations thereof are used interchangeably herein and refer to either qualitative or quantitative determinations, or both qualitative and quantitative determinations. When the terms are used in reference to measurement or detection, any means of assessing the relative amount, including the various methods set forth herein and known in the art.

All publications and patent applications cited in this specification are herein incorporated by reference as if each individual publication or patent application were specifically and individually incorporated herein by reference in their entirety. In case of conflict, the specification, including definitions, will control. The citation of any publication is not to be construed as an admission that the invention is not entitled to antedate such publication by virtue of prior invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

As used herein, the singular forms “a”, “and,” and “the” include plural referents unless the context clearly indicates otherwise. Thus, for example, reference to “an agent” such as an “antibody” or an “inhibitory nucleic acid” or a “small molecule” includes a plurality of such agents, and reference to “an activity or function” can include reference to one or more activities or functions, and so forth.

As used in this specification and the appended claims, the terms “comprise,” “comprising,” “comprises” and grammatical variations of these terms are intended in the non-limiting inclusive sense, that is, to include the particular recited elements or components without excluding any other element or component.

Concentrations used herein, when given in terms of percentages, include weight/weight (w/w), weight/volume (w/v) and volume/volume (v/v) percentages.

As used herein, numerical values are often presented in a range format throughout this document. The use of a range format is merely for convenience and brevity and should not be construed as an inflexible limitation on the scope of the invention. Accordingly, the use of a range expressly includes all possible subranges and all individual numerical values within that range. Furthermore, all numerical values or numerical ranges include integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In addition, reference to a range of 1-5,000 fold includes 1, 2, 3, 4, 5, 6, 7, 8, 9, 10, 11, 12, 13, 14, 15, 16, 17, 18, 19, 20, etc., . . . 5,000 fold, etc., as well as 1.1, 1.2, 1.3, 1.4, 1.5, fold, etc., 2.1, 2.2, 2.3, 2.4, 2.5, fold, etc., and any numerical range within such a ranges, such as 1-2, 3-5, 5-10, 10-50, 50-100, 100-500, 100-1000, 500-1000, 1000-2000, 1000-5000, etc.

As also used herein a series of range formats are used throughout this document. The use of a series of ranges includes combinations of the upper and lower ranges to provide a range. This construction applies regardless of the breadth of the range and in all contexts throughout this patent document. Thus, for example, reference to a series of ranges such as 5 to 10, 10 to 20, 20 to 30, 30, to 50, 50 to 100, 100 to 150, 150 to 200, 200 to 300, or 300 to 400, 400-500, 500-600, or 600-705, includes all combinations of the different ranges such as 5-20, 5-30, 5-40, 5-50, 5-75, 5-100, 5-150, 5-171, and 10-30, 10-40, 10-50, 10-75, 10-100, 10-150, 10-171, and 20-40, 20-50, 20-75, 20-100, 20-150, 20-200, 50 to 200, 50 to 300, 50, to 400, 50 to 500, 100 to 300, 100 to 400, 100 to 500, 100 to 600, 200-400, 200-500, 200 to 600, 200 to 700, and so forth.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include aspects that are not expressly included in the invention are nevertheless disclosed herein.

A number of embodiments of the invention have been described. Nevertheless, it will be understood that various modifications may be made without departing from the spirit and scope of the invention. Accordingly, the following examples, which include data demonstrating a physiological interaction of 4-1BB and Galectin-9 on immune cells, and functional role for 4-1BB-Galectin-9 interactions, are intended to illustrate but not limit the scope of invention described in the claims.

### EXAMPLES

### Example 1

This Example includes data demonstrating that 4-1BB binds to Galectin-9 and 4-1BB Ligand (4-1BBL).

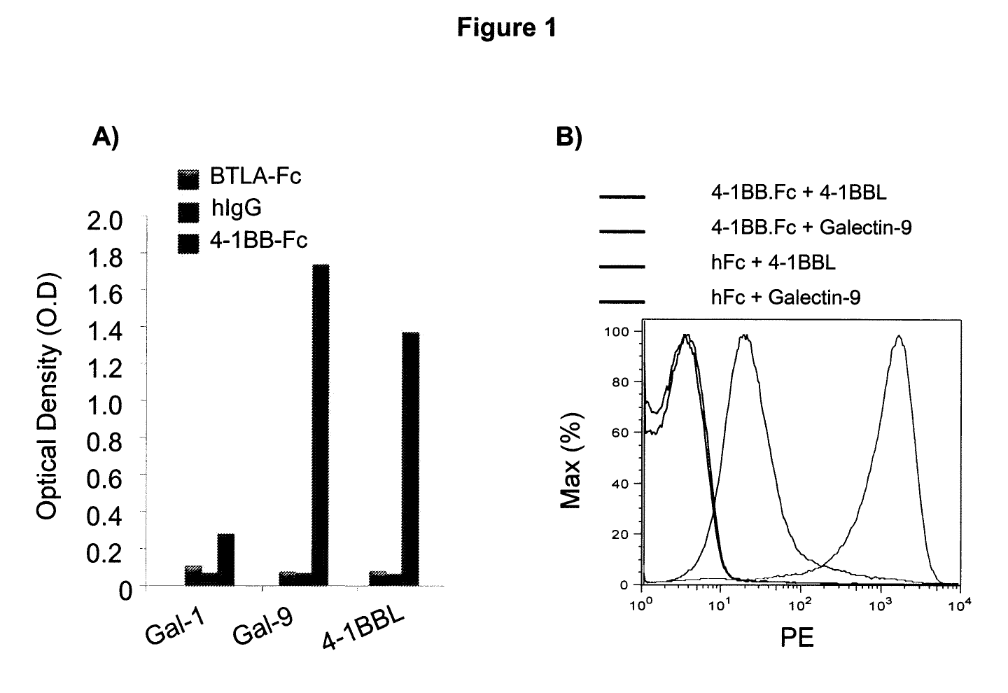

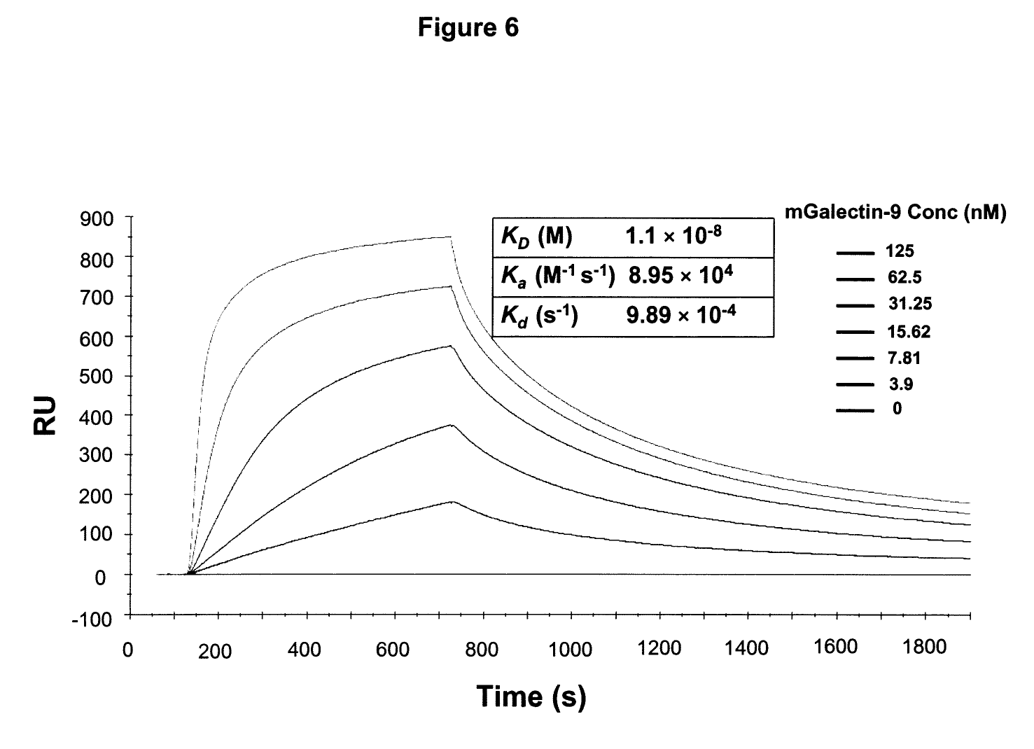

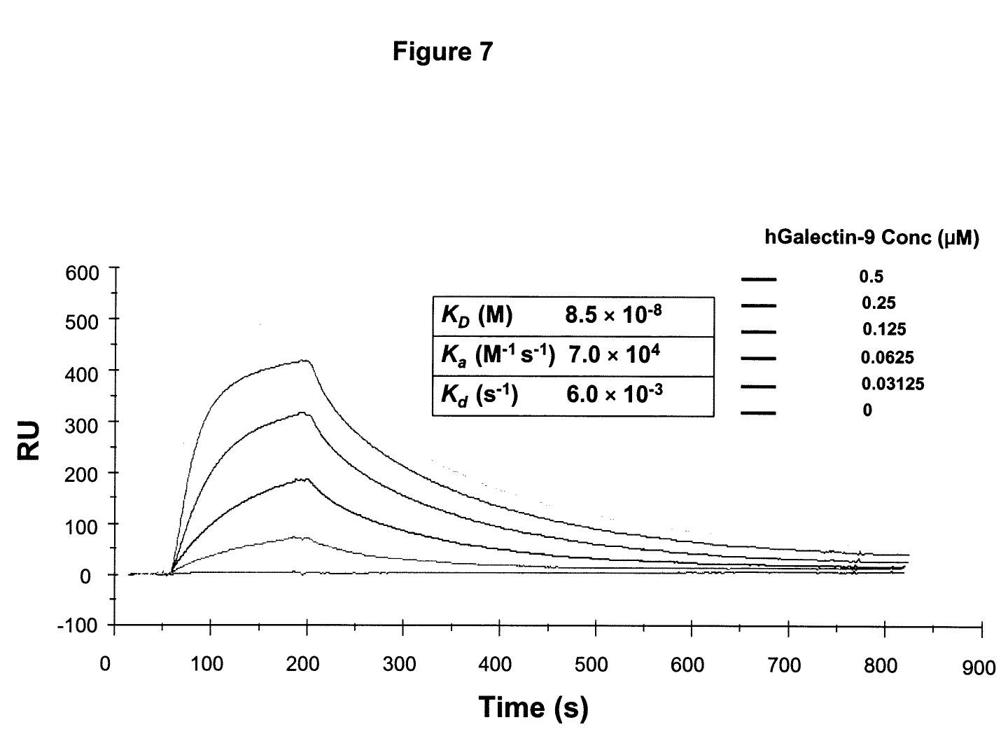

Murine 4-1BB binds both human and murine 4-1BBL, whereas human 4-1BB only binds human 4-1BBL (25). Presented herein is data in ELISA and bead assays showing binding of 4-1BB.Fc (containing the ectodomains of murine 4-1BB) to murine and human Galectin-9. In an ELISA-type assay where recombinant proteins are coated to a plate, binding of 4-1BB.Fc to human Galectin-9 was detected (FIG. 1a). No binding was seen with Galectin-1 as a specificity control, and murine 4-1BBL was used as a further control. Similar results were seen with 4-1BB.Fc coated beads (FIG. 1b). Strong binding between murine 4-1BB and murine Galectin-9 and human 4-1BB and human Galectin-9 was confirmed with Surface Plasmon Resonance, with calculated binding constants of approx 11-85 nM (FIGS. 6 and 7). Collectively these data demonstrate the binding of 4-1BB to Galectin-9 and quantitate the strength of binding.

### Example 2

This Example includes data demonstrating that Galectin 9 and 4-1BBL do not compete for binding to 4-1BB.

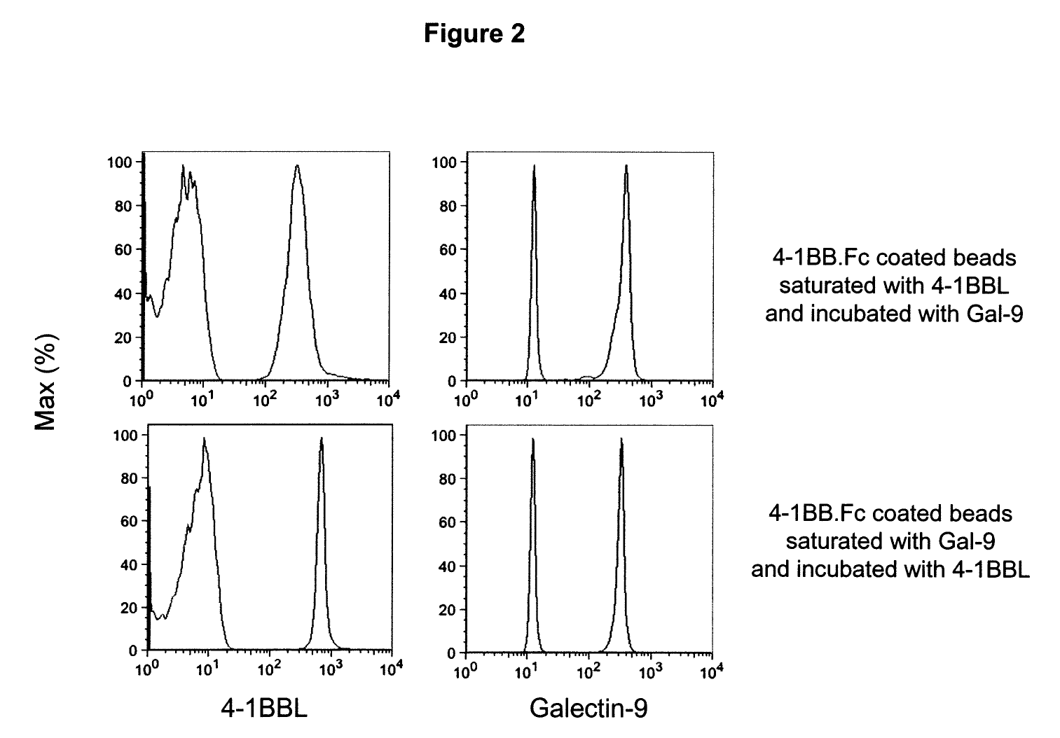

In FIG. 2, Protein G beads were coated with 1 μg of m4-1BB.Fc by incubating in 50 μL of PBS (0.02% Tween 20) at RT for 10 mins. Beads were washed, then incubated at RT for 15 mins with saturating amounts (5 μg) of m4-1BBL or mGalectin-9 in 100 μL PBS (0.02% Tween 20). For competition analysis, 4-1BBL or Galectin-9 pre-incubated beads were washed twice and further incubated with 1 μg of Galectin-9 or 4-1BBL in 50 μL PBS (0.02% Tween 20) respectively at RT for 15 mins. Beads were then washed and stained with APC-anti-4-1BBL and PE-anti-Galectin-9 (blue) or isotype control antibodies (red) and analyzed by flow cytometry. These results demonstrate that there is not competition between Galectin 9 and 4-1BBL for binding to 4-1BB.

### Example 3

This Example includes data demonstrating that binding of soluble Galectin-9 to activated CD4 T cells is dependent on 4-1BB expression and blocked with anti-4-1BB antibodies.

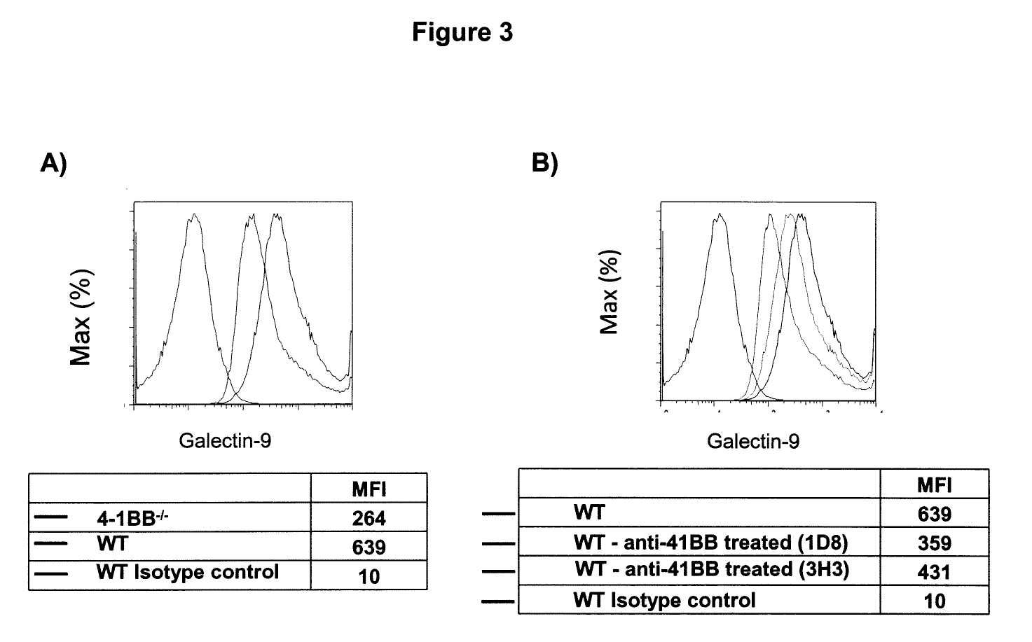

In FIG. 3 MACS purified CD4 T cells from C57BL/6 WT or 4-1BB−/− mice were activated with 3 μg/ml anti-CD3 for 2 days. In FIG. 3A, activated T cells were incubated with recombinant mGalectin-9 at a concentration of 1 μg/million cells in 100 μL PBS at 40 C for 30 mins. Cells were washed, incubated with PE-anti-Galectin-9 at 40 C for 30 mins, then washed and analyzed by flow cytometry for binding of Galectin-9. In FIG. 3B activated WT T cells were preincubated with various anti-4-1BB antibodies (clone 3H3 or 1D8) at a concentration of 1 μg/million cells. Cells were washed and incubated with recombinant mGalectin-9 at a concentration of 1 μg/million cells in 100 μL PBS at 40 C for 30 mins. Cells were washed, incubated with PE-anti-Galectin-9 at 40 C for 30 mins, then washed and analyzed by flow cytometry for binding of Galectin-9. MFI was calculated for Galectin-9 binding. Collectively these results demonstrate that soluble Galectin-9 binding to activated CD4+ T cells is dependent on 4-1BB expression and can be blocked with 4-1BB antibodies.

### Example 4

This Example includes data demonstrating that Galectin 9 binds 4-1BB. 4-1BB can be immunoprecipitated with Galectin-9 from activated T cells, and this endogenous (cis) association is strongly promoted after T cell stimulation.

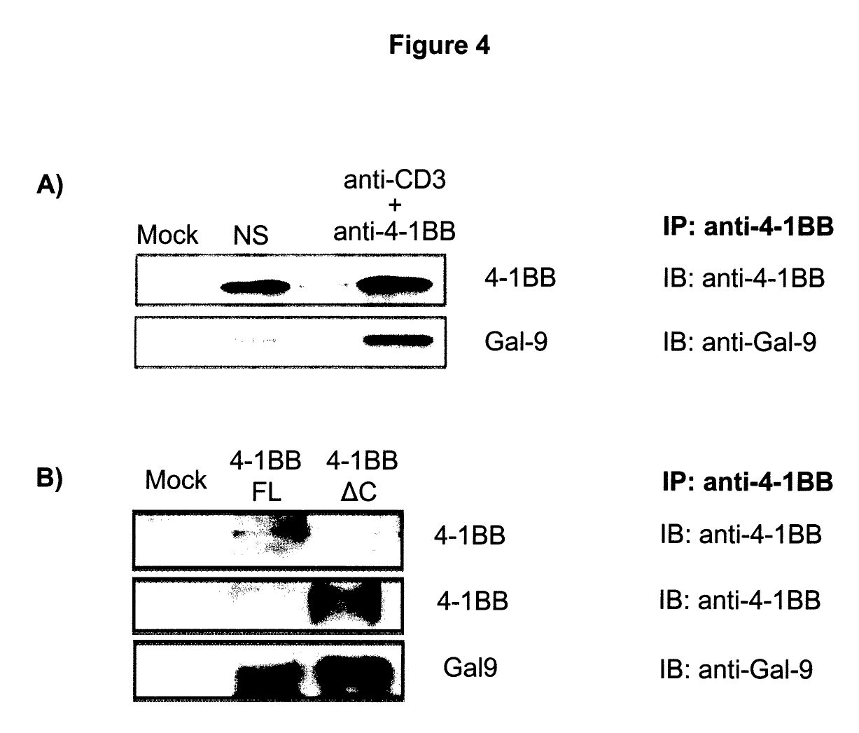

FIG. 4 shows that Galectin-9 co-immunoprecipitates with 4-1BB. In FIG. 4A 4-1BB−/− murine T hybridoma cells were transduced with Myc-tagged m4-1BB, and either stimulated with anti-4-1BB plus anti-CD3 for 15 min, or left unstimulated (NS). Cells were harvested and lysed in 1% NP-40 lysis buffer and 4-1BB was immunoprecipitated using anti-Myc antibody. Precipitates were assessed for 4-1BB and Galectin-9 content by western blot with either anti-4-1BB or anti-Galectin-9. Mock control cells lacking 4-1BB were used as a negative control. In FIG. 4B T hybridoma cells transduced with Myc-tagged full length 4-1BB (FL) or a mutant form of 4-1BB lacking its entire cytoplasmic portion (AC) were lysed in the same buffer as in FIG. 4A. 4-1BB was immunoprecipitated using anti-Myc antibody, followed by western blotting with either anti-4-1BB or anti-Galectin-9 antibody. Collectively, these results demonstrate Galectin-9 binding to 4-1BB in activated T cells.

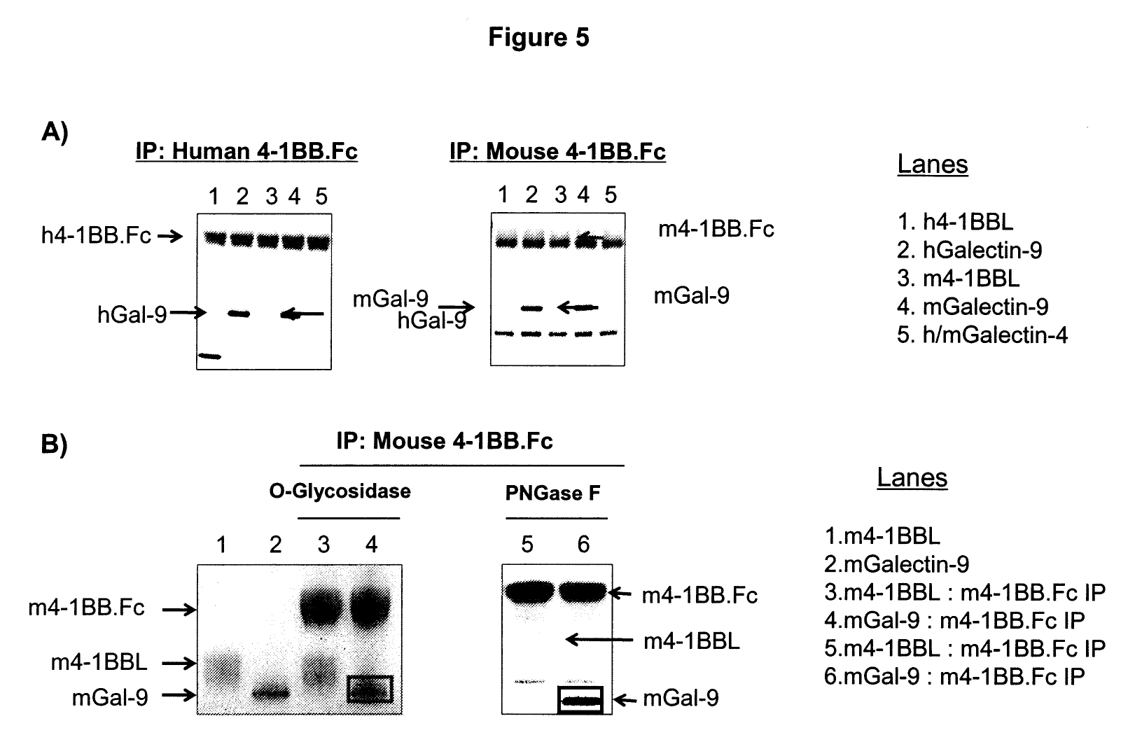

FIG. 5 shows that Galectin-9 is a binding partner for both human and murine 4-1BB, and murine 4-1BB binds to murine Galectin-9 in a carbohydrate independent manner. In FIG. 5A Protein G beads were coated with 5 μg of human or murine 4-1BB.Fc by incubating in 50 μL of PBS (0.02% Tween 20) at RT for 10 mins. Beads were washed, then incubated at RT for 15 mins with 5 μg of various recombinant proteins (human or mouse 4-1BBL, human or mouse Galectin-9 and human or mouse Galectin-4) in 100 μL PBS (0.02% Tween 20). Beads were washed 3× times and bound proteins were eluted and SDS-PAGE was performed. In FIG. 5B elucidates the carbohydrate dependency of mouse 4-1BB binding to mouse Galectin-9. This experiment was performed as in A, except that 4-1BB.Fc was initially treated with either O-Glycosidase or PNGaseF enzymes to remove O-linked or N-linked carbohydrate chains respectively.

### Example 5

This Example includes data demonstrating that Galectin-9 expression is required for 4-1BB signaling to promote immunostimulatory activity in dendritic cells and T cells.

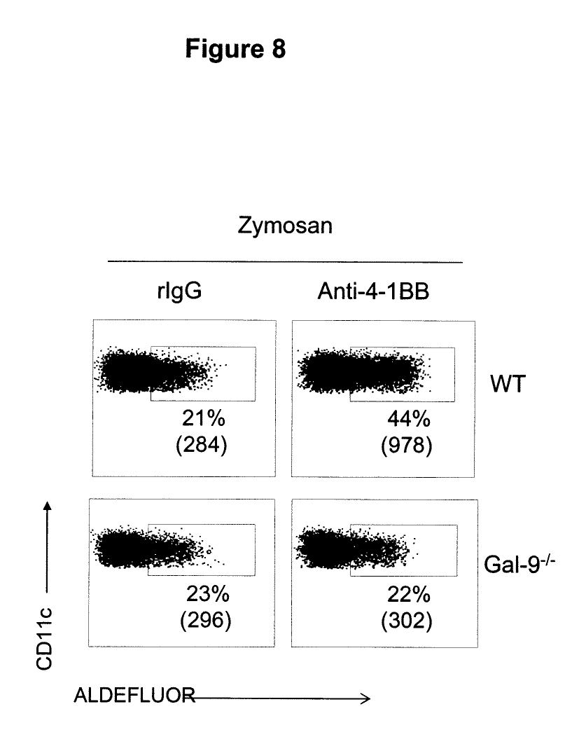

4-1BB can provide immunostimulatory signals to a number of cell types including dendritic cells and T cells (1-6). These signals can promote immune responses against tumors or immune responses against infectious pathogens. Membrane 4-1BB is co-expressed on many of these cells with Galectin-9, suggesting Galectin-9 forms a cis interaction with 4-1BB that allows 4-1BB signaling to induce its immunostimulatory activities. RALDH (retinal dehydrogenase) is the enzyme responsible for retinoic acid production and can be made by dendritic cells and promotes the induction of regulatory T cells (26-27). In wild-type TLR2-stimulated splenic dendritic cells, that also co-express 4-1BB and Galectin-9, 4-1BB signaling induced by an agonist antibody upregulated RALDH expression. However, stimulating 4-1BB did not promote RALDH in dendritic cells lacking Galectin-9 (Galectin-9^{-/-}) even though 4-1BB was still expressed (FIG. 8).

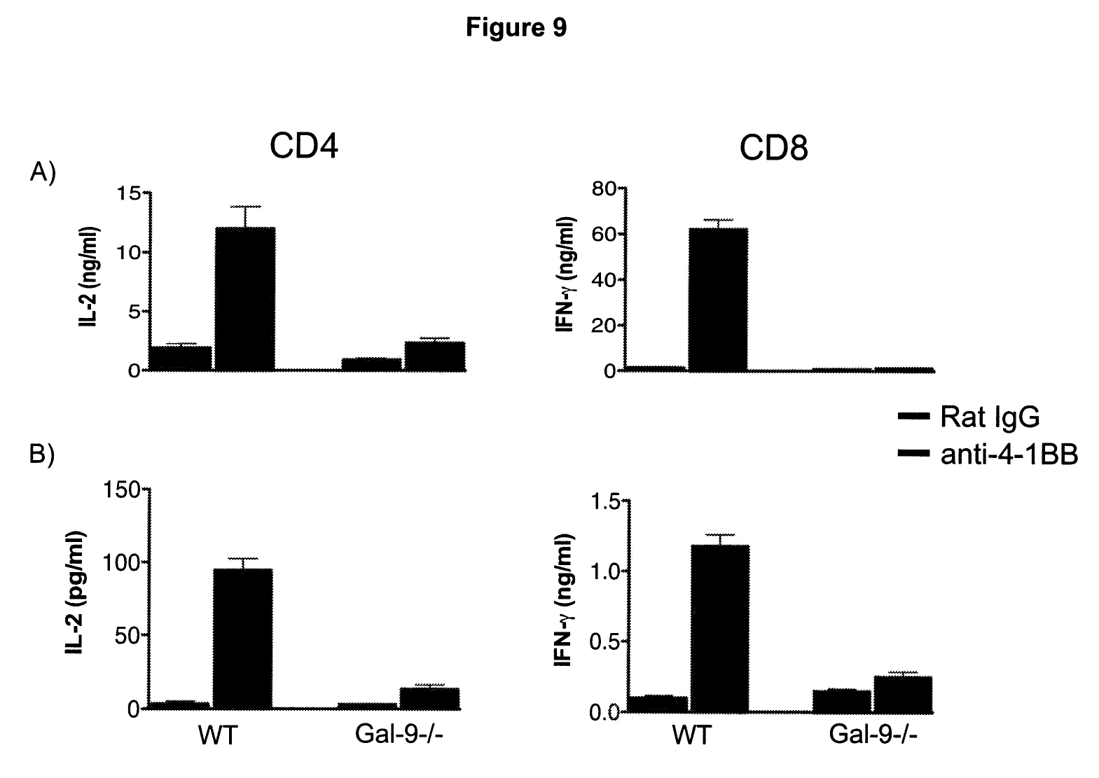

Similar data were obtained in T cells. Galectin-9-deficient CD4 or CD8 T cells that expressed 4-1BB failed to upregulate IL-2 or IFN-γ secretion when stimulated through 4-1BB with an agonist antibody (FIG. 9). These results support the hypothesis that a cis interaction of Galectin-9 and 4-1BB controls 4-1BB functionality and immunostimulatory activity.

### Example 7

This Example includes data demonstrating that 4-1BB mediated anti-inflammatory activity is attenuated in the absence of Galectin-9.

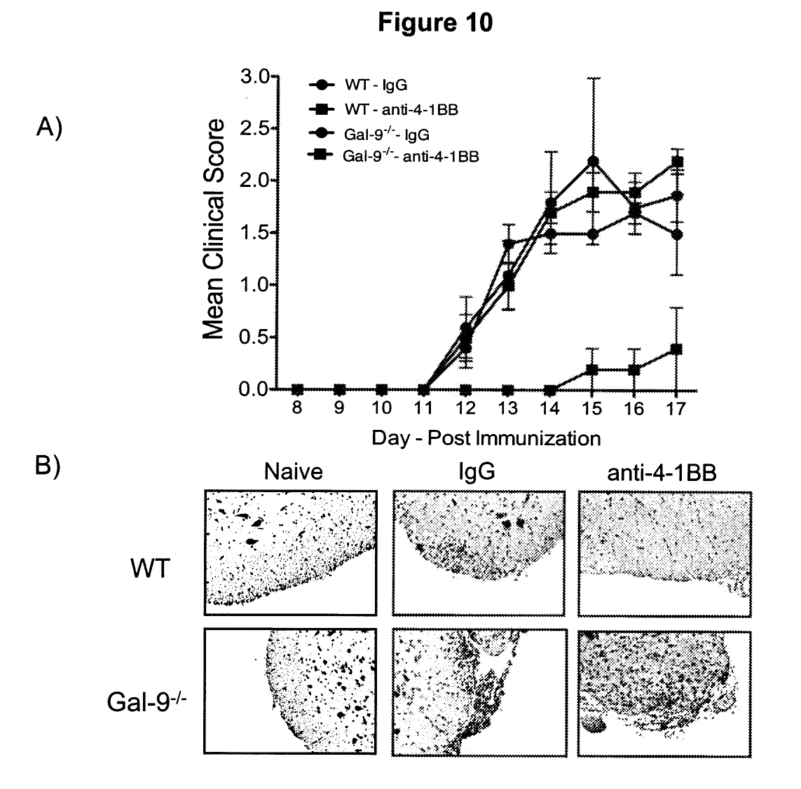

FIGS. 10A and 10B show that anti-4-1BB mediated suppression of EAE is attenuated in the absence of Galectin-9. Agonist antibodies to 4-1BB can suppress inflammation in a number of murine disease models, including experimental autoimmune encephalomyelitis (EAE), and lung inflammation, at least in part through promoting expansion of a regulatory population of CD8 T cells expressing CD11c and making IFN-γ (28-32). To induce EAE disease, 8 week old wild-type (WT) and Galectin-9−/− mice were immunized s.c. at the base of the tail with 100 μg of MOG35-55 peptide (AnaSpec) emulsified in an equal volume of CFA containing 2 mg/ml Mycobacterium Tuberculosis H37 RA (Difco). The mice also received an i.v. injection of 200 ng pertussis toxin (List Biological Laboratories) on day 0 and 2. Animals were injected i.p. with 25 μg agonist anti-4-1BB (clone 3H3) or rat IgG control antibody on day 0, 2, 4 and 8. In FIG. 10A, individual animals were scored daily for clinical signs of EAE using the following criteria: 0, no detectable signs of disease; 0.5, distal limp tail; 1, complete limp tail; 1.5, limp tail and hind limb weakness; 2, unilateral partial hind paralysis; 2.5, bilateral partial hind limb paralysis; 3, complete bilateral hind limb paralysis; 3.5, complete bilateral hind limb paralysis and unilateral forelimb; 4, total paralysis of fore and hind limbs. Mean clinical score was calculated by adding clinical score of individual mice and dividing by the number of mice in each group (n=5). The data show that anti-4-1BB failed to suppress EAE disease in Galectin-9-deficient mice. FIG. 10B shows spinal cords from EAE-induced mice that were dissected, fixed in 4% PFA (Electron Microscopy Sciences), and paraffin embedded. Sections (5 μm) were stained with H&E. To ensure comparable analyses between different groups, six to eight randomly selected sections were analyzed per animal. This data demonstrates that anti-4-1BB suppressed inflammatory cells from infiltrating the spinal cords of wild-type mice but did not inhibit inflammatory cells from accumulating in Galectin-9-deficient mice.

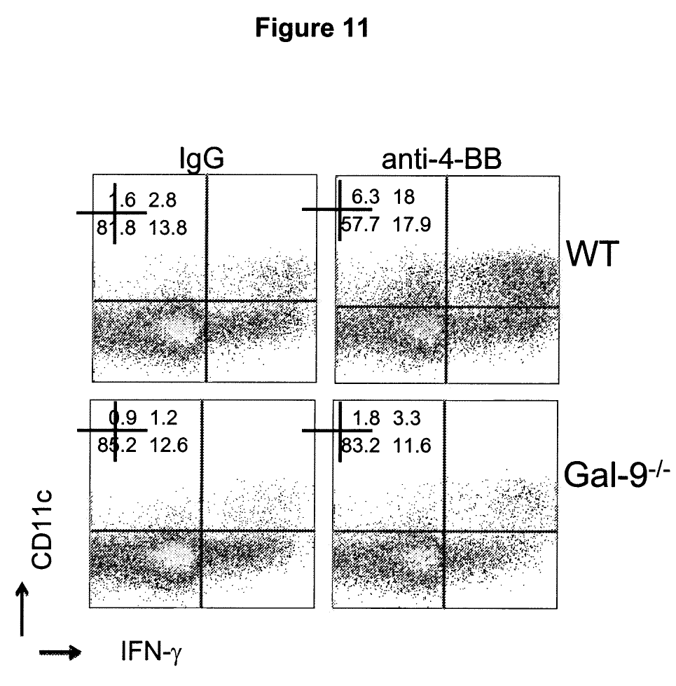

FIG. 11 shows that anti-4-1BB does not promote expansion of suppressive CD8β+CD11c+IFN-γ+ cells in Galectin-9-deficient mice during induction of EAE. WT and Gal-9−/− mice were immunized to induce EAE as in FIG. 10, and injected with control IgG or agonist anti-4-1BB also as in FIG. 10. Draining lymph nodes cells were harvested on day 15 and stimulated with 50 ng/ml PMA and 500 ng/ml ionomycin in medium containing Brefeldin A for 5 hrs. Cells were stained with PerCP-Cy5.5-anti-CD8β, FITC-anti-CD11c, and intracellular APC-IFN-γ and analyzed by flow cytometry. Plots are gated CD8β+ cells, analyzed for CD11c and IFN-γ. Data representative of 5 animals from each group. Percent positive in each quadrant indicated.

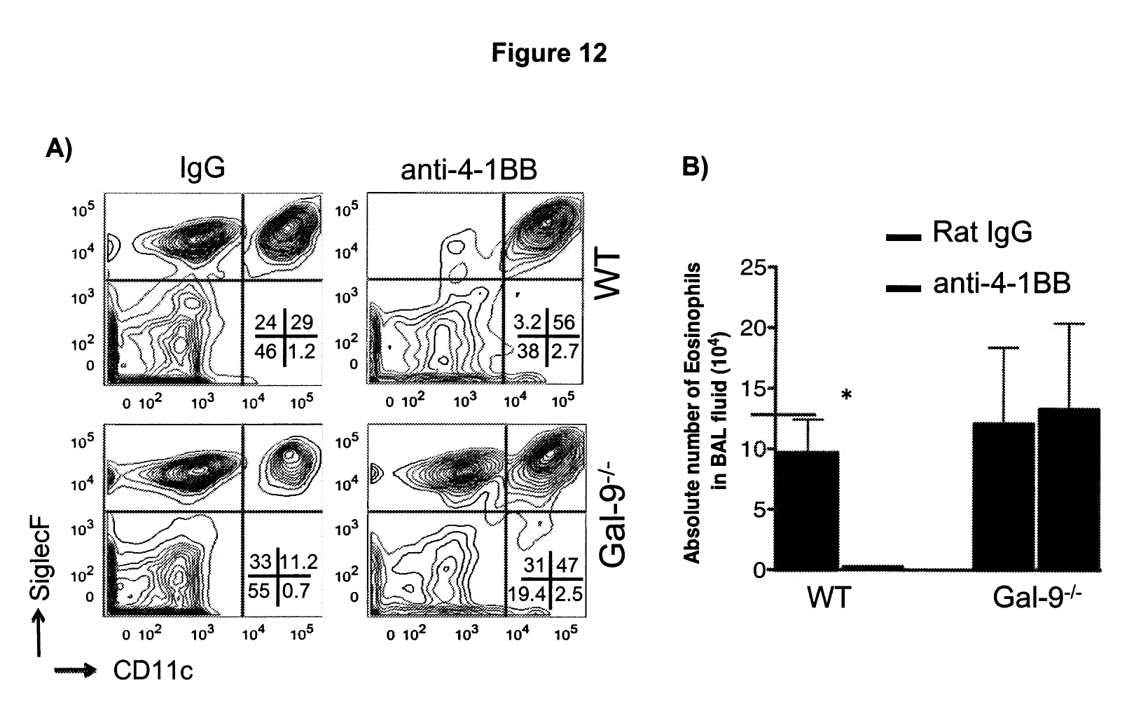

FIG. 12 shows that anti-4-1BB mediated suppression of asthmatic eosinophilic lung inflammation is attenuated in the absence of Galectin-9. To induce asthmatic lung inflammation, 8 week old C57BL/6 WT or Galectin-9−/− mice were immunized i.p. with 20 μg OVA (Sigma-Aldrich) adsorbed to 2 mg of aluminum hydroxide and magnesium hydroxide (Alum; Fischer Scientific International) on days 0 and 7. Mice were challenged with 20 μg OVA in 20 μl PBS given intranasally on days 14-16. Animals were injected i.p. with 200 μg agonist anti-4-1BB (clone 3H3) or control rat IgG 1 day before the first OVA immunization. Mice were sacrificed on day 18, and bronchoalveolar lavage (BAL) of the lungs was performed. Cells in the lavage fluid were counted using a hemocytometer, and then stained with APC-anti-CD11c and PE-anti-SiglecF and analyzed by flow cytometry. FIG. 12A shows representative plots of CD11c versus SiglecF, with eosinophils being SiglecF+CD11c−. FIG. 12B shows the absolute number of Eosinophils (SiglecF+CD11c−) from each animal, calculated based on the percentages obtained by flow cytometry. Data represents mean±SEM of 4 animals per group.

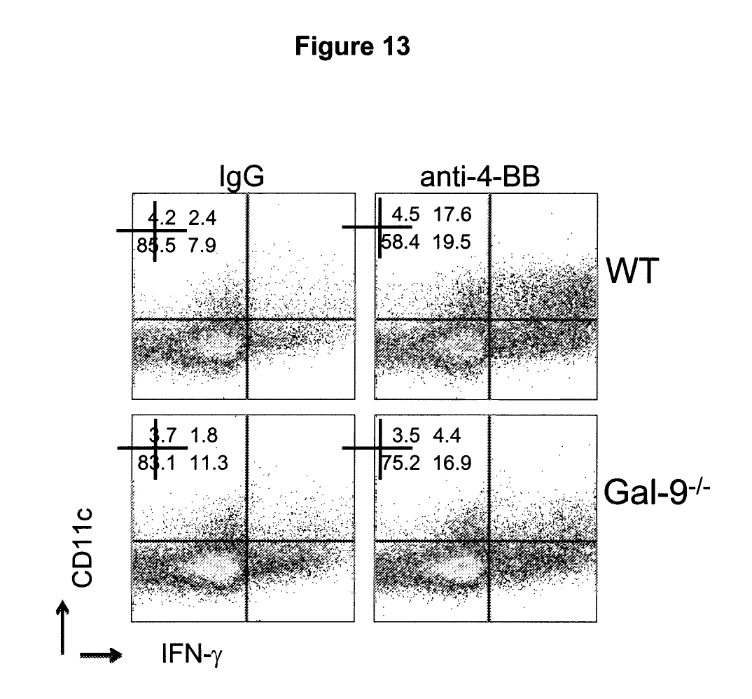

FIG. 13 shows that anti-4-1BB does not promote expansion of suppressive CD8β+CD11c+IFN-γ+ cells in Galectin-9-deficient mice during induction of asthmatic lung inflammation. WT and Gal-9−/− mice were immunized to induce asthmatic lung inflammation as in FIG. 12, and injected with control IgG or agonist anti-4-1BB also as in FIG. 12. On day 18, lung draining lymph node cells were stimulated with 50 ng/ml PMA and 500 ng/ml ionomycin in medium containing Brefeldin A for 5 hrs. Cells were stained with PerCP-Cy5.5-anti-CD8β and FITC-anti-CD11c, and APC-anti-IFN-γ and analyzed by flow cytometry. Plots are gated CD8β+ cells, analyzed for CD11c and IFN-γ. Data representative of 4 animals from each group. Percent positive in each quadrant indicated. Collectively, these results demonstrate attenuation of 4-1BB mediated anti-inflammatory activity in the absence of Galectin-9.

### REFERENCES

1. Croft, M. Co-stimulatory members of the TNFR family: Keys to effective T-cell immunity. Nat Rev Immunol. 2003. 3:609-20.
2. Watts, T. H. TNF/TNFR family members in costimulation of T cell responses. Annu Rev Immunol. 2005. 23:23-68.
3. Croft, M. The role of TNF superfamily members in T-cell function and diseases. Nat Rev Immunol. 2009. 9:271-85. PMCID: PMC2737409
4. Vinay, D. S., Cha, K., and Kwon, B. S. Dual immunoregulatory pathways of 4-1BB signaling. J Mol Med. 2006. 84:726-36.
5. So, T., Lee, S. W., and Croft, M. Immune regulation and control of regulatory T cells by OX40 and 4-1BB. Cytokine Growth Factor Rev. 2008. 19:253-62. PMCID: PMC2486494
6. Lee, S. W., and Croft, M. 4-1BB as a Therapeutic Target for Human Disease. Adv Exp Med Biol. 2009. 647:120-9. PMCID: PMC n/a
7. Wada, J., and Kanwar, Y. S. Identification and characterization of galectin-9, a novel beta-galactosid-ebinding mammalian lectin. J Biol Chem. 1997. 272:6078-86.
8. Wada, J., Ota, K., Kumar, A., Wallner, E. I., and Kanwar, Y. S. Developmental regulation, expression, and apoptotic potential of galectin-9, a beta-galactoside binding lectin. The Journal of clinical investigation. 1997. 99:2452-61.
9. Rabinovich, G. A., Baum, L. G., Tinari, N., Paganelli, R., Natoli, C., Liu, F. T., and Iacobelli, S. Galectins and their ligands: amplifiers, silencers or tuners of the inflammatory response? Trends in immunology. 2002. 23:313-20.
10. Rabinovich, G. A., Liu, F. T., Hirashima, M., and Anderson, A. An emerging role for galectins in tuning the immune response: lessons from experimental models of inflammatory disease, autoimmunity and cancer. Scandinavian journal of immunology. 2007. 66:143-58.
11. Liu, F. T. Regulatory roles of galectins in the immune response. Int Arch Allergy Immunol. 2005. 136:385-400.
12. Liu, F. T., and Rabinovich, G. A. Galectins as modulators of tumour progression. Nature reviews. 2005.5:29-41.
13. Hsu, D. K., Chen, H. Y., and Liu, F. T. Galectin-3 regulates T-cell functions. Immunol Rev. 2009. 230:114-27.
14. Rabinovich, G. A., and Toscano, M. A. Turning ‘sweet’ on immunity: galectin-glycan interactions in immune tolerance and inflammation. Nat Rev Immunol. 2009. 9:338-52.
15. Zhu, C., Anderson, A. C., Schubart, A., Xiong, H., Imitola, J., Khoury, S. J., Zheng, X. X., Strom, T. B., and Kuchroo, V. K. The Tim-3 ligand galectin-9 negatively regulates T helper type 1 immunity. Nat Immunol. 2005. 6:1245-52.
16. Katoh, S., Ishii, N., Nobumoto, A., Takeshita, K., Dai, S. Y., Shinonaga, R., Niki, T., Nishi, N., Tominaga, A., Yamauchi, A., and Hirashima, M. Galectin-9 inhibits CD44-hyaluronan interaction and suppresses a murine model of allergic asthma. Am J Respir Crit Care Med. 2007. 176:27-35.
17. Kashio, Y., Nakamura, K., Abedin, M. J., Seki, M., Nishi, N., Yoshida, N., Nakamura, T., and Hirashima, M. Galectin-9 induces apoptosis through the calcium-calpain-caspase-1 pathway. J Immunol. 2003. 170:3631-6.
18. Wang, F., He, W., Thou, H., Yuan, J., Wu, K., Xu, L., and Chen, Z. K. The Tim-3 ligand galectin-9 negatively regulates CD8+ alloreactive T cell and prolongs survival of skin graft. Cell Immunol. 2007. 250:68-74.
19. Fukushima, A., Sumi, T., Fukuda, K., Kumagai, N., Nishida, T., Okumura, K., Akiba, H., Yagita, H., and Ueno, H. Roles of galectin-9 in the development of experimental allergic conjunctivitis in mice. Int Arch Allergy Immunol. 2008. 146:36-43.
20. Nagahara, K., Arikawa, T., Oomizu, S., Kontani, K., Nobumoto, A., Tateno, H., Watanabe, K., Niki, T., Katoh, S., Miyake, M., Nagahata, S., Hirabayashi, J., Kuchroo, V. K., Yamauchi, A., and Hirashima, M. Galectin-9 increases Tim-3+ dendritic cells and CD8+ T cells and enhances antitumor immunity via galectin-9-Tim-3 interactions. Journal of immunology. 2008. 181:7660-9.
21. Seki, M., Oomizu, S., Sakata, K. M., Sakata, A., Arikawa, T., Watanabe, K., Ito, K., Takeshita, K., Niki, T., Saita, N., Nishi, N., Yamauchi, A., Katoh, S., Matsukawa, A., Kuchroo, V., and Hirashima, M. Galectin-9 suppresses the generation of Th17, promotes the induction of regulatory T cells, and regulates experimental autoimmune arthritis. Clin Immunol. 2008. 127:78-88.
22. Niwa, H., Satoh, T., Matsushima, Y., Hosoya, K., Saeki, K., Niki, T., Hirashima, M., and Yokozeki, H. Stable form of galectin-9, a Tim-3 ligand, inhibits contact hypersensitivity and psoriatic reactions: a potent therapeutic tool for Th1- and/or Th17-mediated skin inflammation. Clin Immunol. 2009. 132:184-94.
23. Nobumoto, A., Nagahara, K., Oomizu, S., Katoh, S., Nishi, N., Takeshita, K., Niki, T., Tominaga, A., Yamauchi, A., and Hirashima, M. Galectin-9 suppresses tumor metastasis by blocking adhesion to endothelium and extracellular matrices. Glycobiology. 2008. 18:735-44.
24. Su, E. W., Bi, S., and Kane, L. P. Galectin-9 Regulates T Helper Cell Function Independently of Tim-3. Glycobiology. 2010. 21:1258-65.
25. Bossen, C., Ingold, K., Tardivel, A., Bodmer, J. L., Gaide, O., Hertig, S., Ambrose, C., Tschopp, J., and Schneider, P. Interactions of tumor necrosis factor (TNF) and TNF receptor family members in the mouse and human. J Biol Chem. 2006. 281:13964-71.
26. Sun, C. M., Hall, J. A., Blank, R. B., Bouladoux, N., Oukka, M., Mora, J. R., and Belkaid, Y. Small intestine lamina propria dendritic cells promote de novo generation of Foxp3 T reg cells via retinoic acid. J Exp Med. 2007. 204:1775-85. PMCID: PMC2118682.
27. Coombes, J. L., Siddiqui, K. R., Arancibia-Carcamo, C. V., Hall, J., Sun, C. M., Belkaid, Y., and Powrie, F. A functionally specialized population of mucosal CD103+ DCs induces Foxp3+ regulatory T cells via a TGFbeta and retinoic acid-dependent mechanism. J Exp Med. 2007. 204:1757-64.
28. Seo, S. K., Choi, J. H., Kim, Y. H., Kang, W. J., Park, H. Y., Suh, J. H., Choi, B. K., Vinay, D. S., and Kwon, B. S. 4-1BB-mediated immunotherapy of rheumatoid arthritis. Nat. Med. 2004. 10:1088-94.
29. Choi, B. K., Asai, T., Vinay, D. S., Kim, Y. H., and Kwon, B. S. 4-1BB-mediated amelioration of experimental autoimmune uveoretinitis is caused by indoleamine 2,3-dioxygenase-dependent mechanisms. Cytokine. 2006. 34:233-42.
30. Sun, Y., Lin, X., Chen, H. M., Wu, Q., Subudhi, S. K., Chen, L., and Fu, Y. X. Administration of agonistic anti-4-1BB monoclonal antibody leads to the amelioration of experimental autoimmune encephalomyelitis. J. Immunol. 2002. 168:1457-65.
31. Vinay, D. S., Kim, J. D., and Kwon, B. S. Amelioration of mercury-induced autoimmunity by 4-1BB. J. Immunol. 2006. 177:5708-17.
32. Vinay, D. S., Kim, C. H., Choi, B. K., and Kwon, B. S. Origins and functional basis of regulatory CD11c+CD8+ T cells. Eur. J. Immunol. 2009. 39:1552-63. PMCID: PMC2748059.

1. A method of modulating an immune response, comprising contacting 4-1BB or a galectin with an agent that modulates binding of 4-1BB to the galectin, thereby modulating an immune response.

2.-3. (canceled)

4. The method of claim 1, wherein the agent decreases, reduces, inhibits, suppresses or disrupts binding of 4-1 BB to the galectin.

5. The method of claim 1, wherein the agent increases, enhances, stimulates, or promotes binding of 4-1BB to the galectin.

6. The method of claim 1, wherein the galectin is Galectin-9.

7. The method of claim 1, wherein the agent binds to 4-1BB, binds to the galectin, binds to 4-1BB ligand (4-1BBL), binds to Tim-3 or binds to CD44.

8. The method of claim 1, wherein the agent comprises an antibody or an antibody fragment thereof that binds to 4-1BB, the galectin, 4-1BB ligand (4-1BBL), Tim-3 or CD44.

9.-17. (canceled)

18. The method of claim 1, wherein the agent comprises a peptide or a fragment of a 4-1BB, galectin, Galectin-9, 4-1BB ligand (4-1BBL), Tim-3 or CD44 polypeptide sequence that binds to 4-1BB, galectin, 4-1BB ligand (4-1BBL), Tim-3 or CD44 polypeptide sequence.

19.-32. (canceled)

33. The method of claim 1, wherein the agent comprises an inhibitory nucleic acid that reduces expression or activity of 4-1BB, the galectin, 4-1BB ligand, Tim-3 or CD44.

34.-36. (canceled)

37. The method of claim 1, wherein the agent comprises an aptamer.

38.-39. (canceled)

40. The method of claim 1, wherein the agent comprises a fusion polypeptide or a chimeric polypeptide.

41. The method of claim 1, wherein the agent comprises a small molecule.

42. The method of claim 1, wherein the method comprises decreasing, reducing, inhibiting, suppressing, limiting or controlling an undesirable or aberrant immune response, disorder or disease, an inflammatory response, disorder or disease, inflammation, or an autoimmune response, disorder or disease, or an adverse symptom of an undesirable or aberrant immune response, disorder or disease, an inflammatory response, disorder or disease, inflammation, or an autoimmune response, disorder or disease.

43. The method of claim 1, wherein the method comprises increasing, stimulating, enhancing, promoting, inducing or activating an immune response, inflammatory response or inflammation.

44. A method of modulating an immune response in a subject, comprising administering an agent that modulates binding of 4-1BB to a galectin to the subject, thereby modulating the immune response in the subject.

45. The method of claim 44, wherein the subject has or has had an undesirable or aberrant immune response, disorder or disease, an inflammatory response, disorder or disease, inflammation, or an autoimmune response, disorder or disease or an adverse symptom of an undesirable or aberrant immune response, disorder or disease, an inflammatory response, disorder or disease, inflammation, or an autoimmune response, disorder or disease.

46.-47. (canceled)

48. The method of claim 45, wherein the undesirable or aberrant immune response, disorder or disease, inflammatory response, disorder or disease, inflammation, or autoimmune response, disorder or disease comprises rheumatoid arthritis, juvenile rheumatoid arthritis, osteoarthritis, psoriatic arthritis, multiple sclerosis (MS), encephalomyelitis, myasthenia gravis, systemic lupus erythematosus (SLE), asthma, allergic asthma, autoimmune thyroiditis, atopic dermatitis, eczematous dermatitis, psoriasis, Sjögren's Syndrome, Crohn's disease, aphthous ulcer, iritis, conjunctivitis, keratoconjunctivitis, ulcerative colitis (UC), inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus (IDDM, type I diabetes), insulin-resistant diabetes mellitus (type 2 diabetes), immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common γ chain (γc) deficiency, Janus-associated kinase 3 (JAK3) deficiency and reticular dysgenesis; primary T cell immunodeficiency such as DiGcorge syndrome, Nude syndrome, T cell receptor deficiency, MHC class II deficiency, T AP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (γ5/14.1) deficiency, Hyper-IgM syndrome: X-linked (CD40 ligand deficiency) or non-X-Iinked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon γ receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1A/SAP deficiency), hyper IgE syndrome or Graft vs. Host Disease (GVHD).

49. The method of claim 44, wherein the immune response or inflammatory response is an anti-cancer or anti-pathogen immune response or inflammatory response.

50.-74. (canceled)
